(19) United States
(12) Patent Application Publication
(10) Pub. No.: US 2026/0317388 A1
(43) Pub. Date: Oct. 15, 2026

(54) SELECTIVE NUCLEAR BLOCKADE FOR THE TREATMENT OF SEPSIS

(71) Applicants: VANDERBILT UNIVERSITY, Nashville, TN (US); THE UNITED STATES GOVERNMENT AS REPRESENTED BY THE DEPARTMENT OF VETERANS AFFAIRS, Washington, DC (US)

(72) Inventors: Jack Jacek HAWIGER, Nashville, TN (US); Jozef ZIENKIEWICZ, Nashville, TN (US); Huan QIAO, Nashville, TN (US); Yan LIU, Nashville, TN (US)

(21) Appl. No.: 19/483,087

(22) PCT Filed: May 9, 2024
(86) PCT No.: PCT/US2024/028662

§ 371 Date: Nov. 10, 2025

Related U.S. Application Data
(60) Provisional application No. 63/501,087, filed on May 9, 2023.
(60) Provisional application No. 63/517,769, filed on Aug. 4, 2023.

(51) Int. Cl. C07K 14/50; A61K 38/00; A61P 29/00

(52) U.S. Cl. CPC C07K 14/50; A61P 29/00; A61K 38/00; C07K 2319/02; C07K 2319/09

(57) ABSTRACT

Disclosed are compositions and methods for treating microbial inflammation including its end-stage sepsis and reversing changes in gene expression resulting from microbial inflammation. In one aspect, the compositions and methods disclosed herein can be used to reduce the microbial lode from infected tissues, organs, or systems in a subject.

### CROSS REFERENCE TO RELATED APPLICATIONS

This application claims the benefit of U.S. Provisional Application No. 63/501,087, filed on May 9, 2023 and U.S. Provisional Application No. 63/517,769 filed on Aug. 4, 2023, Applications which are incorporated herein by reference in their entirety.

### STATEMENT OF GOVERNMENT SUPPORT

This invention was made with government support under Grant No. 101 BX002750 awarded by the U.S. Department of Veteran Affairs. The government has certain rights in the invention.

### BACKGROUND

Sepsis, a life-threatening complication of out-of-control infections, is rising globally. This overwhelming microbial inflammation encompassing multiple systems is caused by viruses (e.g., influenza, Ebola, Dengue, and SARS Coronavirus 2), Gram-positive and negative bacteria (e.g., Staphylococci, Streptococci, E. coli, Pseudomonas, and those causing wound infections in the battlefield), yeasts (e.g., Candida), and protozoa (e.g., Malaria). Severe infections evolving into sepsis, and then into septic shock, have become a pressing, worldwide public health problem that continues to grow in magnitude as the global population ages. In the US, at least 1.7 million adults develop sepsis each year and 350,000 die of the illness. While one in three patients who died in the U.S. hospitals had sepsis, its mortality approaches that of heart attacks and exceeds the number of deaths from stroke. Neonates and the elderly constitute the most vulnerable to sepsis patients, with a 28-days mortality rate exceeding 40%. Thus, the prevention and very early control of severe viral, bacterial, fungal, and protozoal infections remains crucial for taming their progression into sepsis. Survivors suffer moderate to severe cognitive impairment as compared to other patients discharged from Intensive Care Units. In addition to the human cost of sepsis, its expenses rank highest among admissions for all diseases in the U.S. hospitals. They do not include expenses for the long-term care of survivors who suffer persistent developmental abnormalities (after neonatal sepsis) and incapacitating cognitive decline (after adult sepsis). Thus, new approaches to the adjunctive therapy of sepsis are needed.

### SUMMARY

Disclosed are methods and compositions related to the modulation of gene expression to reverse and/or inhibit changes in gene expression during microbial inflammation.

Also disclosed herein are methods of reversing the expression profile (i.e., decreasing, reducing, or inhibiting expression when the expression profile increases or increasing expression when the expression profile decreases) of one or more genes encoding mediators of inflammation in a subject whose gene expression modulates during microbial inflammation (including, but not limited to sepsis) as a result of microbial infection (such as, for example, a viral, bacterial, fungal, or parasitic infection) comprising administering to the subject a therapeutically effective amount of a nuclear transport checkpoint inhibitor (NTCI) (such as, for example, SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41).

In one aspect, disclosed herein are methods of reversing the expression profile of one or more genes encoding mediators of inflammation of any preceding aspect, wherein the one or more genes are selected from the group consisting of I110, Acod1, Saa3, Gpr31b, Fgf23, Igfbp1, Mrgpra2b, Ccl3, Ccl4, Gpr84, Adamts4, Madcam1, Olfm4, Il1f9, Gpr31a, Gpr31c, Ptx3, Slc7a11, Gm6553, Tmprss11g, Tac1, Il1f6, Ccl2, Psg16, Fos11, Sprr2a2, Serpina3m, Il1rn, Ccl20, Tnf, Gm43181, Clec4e, Gm45774, Ugtla10, Kcnt1, Mt1, Mefv, Clec4d, Ms4a4a, Btbd16, Spic, Orm1, Serpina3f, Il1b, Serpina10, 9830107B12Rik, Gm49839, Msr1, Ankrd2, Saa4, S100a8, Hpx, Apoa4, Steap4, Cxcl10, S100a9, Wfdc17, Gnat1, Fgg, Fgb, Lilr4b, 2010003K11Rik, Serpina3n, AA467197, Gm16685, Timp1, Fga, Ifitm6, Lcn2, F10, Leap2, Retnlg, Ccl7, Ikbke, Vcan, F13a1, Serpina3g, 1200007C13Rik, Ngp, Siglece, Lrrc25, Gm10309, Trem1, Slc39a14, Fgl1, 1127, Tcp10b, Serpina3i, Cxcl9, A730049H05Rik, Gm8883, Fpr2, Saa2, Saa1, Prok2, B230303A05Rik, Gm43079, Gm49383, Cldn14, Gm15056, U90926, Stfa2, Prokr2, Lbx1, Cstdc5, Tarm1, Mrgpra2a, Cstdc4, Gm48786, Gm19510, Gm32089, Gm5966, Stfa3, Tmem132e, Orm2, Gm28551, A630023A22Rik, Gm7457, Serpina7, Bmerb1, Klk9, 1700011B04Rik, Asprv1, Dmbt1, Tmprss11d, Slc5a7, Ifitm7, Gm38340, Pglyrp3, Gm26748, 4930431P03Rik, 4732490B19Rik, Sectm1b, Gm4948, Gm20406, Ras110b, Gm36079, Sult2a3, Apcs, Mymk, Nkx2-9, Gm26535, Rnase2a, Slc22a20, Stfa211, Gm4949, Gm11738, Gm29686, Serpine3, Gm807, Gm26444, Trdv5, Sultle1, Serpina3e-ps, 4930578G10Rik, Gm37645, Ceacam19, Ackr1, Ms4a3, B130046B21Rik, Ctcflos, Doc2b, Gm49573, Gm38275, Tex44, Gpr22, Ceacam10, E030018B13Rik, Gm37131, Htr3a, Slc13a5, Nxf3, Gm49427, Pde6h, Ubd, Fcrlb, Sult2a5, Tat, Gm6610, Gm44292, Gm44165, Prss22, Gm11714, Gm49368, Gm48065, Aoah, Tctex1d1, Phox2a, Slc36a3, Sult2a1, Gm37347, Gm50083, Epx, Gm26965, Reg3g, Cela3b, Gm12503, Gpbar1, Dnah6, Fut2, Muc4, Gm43189, Angpt17, Pcdh8, Sftpa1, Mlana, Gm16350, Syt2, Gm17055, Ppy, Ccdc33, Gm33280, Gm45643, Lrit3, Sbk2, Slc6a19os, Slamf1, Gm5541, Fgf21, Gm49392, Slc6a16, Olr1, Gm35028, Slfn4, Gm20548, Gm40709, Speer4f2, Gm13363, Egr2, Gm44260, Marc1, Crygn, Lpo, Gabrp, Bpifc, Fgf18, Lncenc1, Prss27, Muc19, Gm39929, Gm2670, 5830487J09Rik, Olfr1442, Krt20, Gm19684, Gm33424, 4732419C18Rik, Gm37621, Creb313, Gm14319, Dnah10, Areg, Armc12, Eif4ebp3, Gdf6, Gm49037, Gm42793, Der13, Nptx2, Atp12a, Kcna7, Prg4, Ctla4, Gm44164, Mmp7, Gm44949, Slc17a8, Lamb3, Plekhs1, Trim17, Dusp8, 4930422C21Rik, Gm10727, Col27a1, Tacc2, Fat2, Lif, Klk1b1, Tsc22d1, Wnt8b, Grem1, Hesx1, Hhipl1, Ugtla5, Klk1b4, Gm8947, A530013C23Rik, Mylk4, Cyp2a5, Muc1, Gm44588, Gm45053, Ano5, Gsg11, Spon2, Oxtr, Rnf112, Ppp2r2c, Crabp1, St6galnac3, Agtr2, Gjc2, Gm33195, Gabra4, Aplnr, E030013I19Rik, Pcdh12, Chp2, Gng8, Pla2g2d, Gm20683, Mansc4, Gm10824, Ces2b, Kank4, Zmat4, Cdr1os, Wnt2, Gm17040, Wfdc16, Rab42, Tbxa2r, Plppr5, Misp3, Mmp28, Lrrn4, Sost, Tmem200a, Shisa2, Stmn2, Chrm1, Tmeff2, 2810433D01Rik, Pabpc41, Dact3, Mett124, Gpr34, D430019H16Rik, Hoxc5, Tekt5, Megf10, Wscd1, Gm32219, Tril, Agtr1b, 2610203C22Rik, Abcg3, Star, Grik2, Grin2c, Vstm5, Smad9, Pianp, Rnf144a, Cthrc1, Nxpe4, Mrc2, Cx3cr1, I15ra, Kcnc4, Fam163a, Cxcr3, A330074K22Rik, Gm11627, Mmp11, Fam181b, Cd209b, Tnfrsf19, Sept4, Nlgn3, Baalc, Ngb, Nxph3, Klb, Igkv4-72, Ptprv, Nrep, Itga8, Myct1, Palm, Syt3, Tspan18, Piezo2, Nat8f3, Cngb1, Cd209g, Faim2, Gfra2, Srpk3, Gm3716, Klhl13, Mogat2, Tmem233, Duxb13, Rnase1, Defb42, AL935121.1, Zp1, Snurf, Gm25541, 4933403008Rik, 1810046K07Rik, Gm5639, Myh2, Gm765, Gm50242, Gm6018, Ackr4, Tnfrsf17, Gm37858, Igkv14-130, Ptprh, Gpr165, Gm47652, Gm16534, 0610012D04Rik, Gm36569, Gzmk, Gm9918, Gm33677, St6gal2, Krtap17-1, Olfr1342, Lhcgr, Ccdc42, Zfp985, Igkv4-70, Gm31517, Gm9767, Olfr71, Slc26a3, Trpv3, Gm49727, Gm43951, E330018M18Rik, Calcb, Rspo2, Glp1r, Fam124b, Amtn, 4930570G19Rik, Gm11574, Pla2g2e, Sfrp2, Pigz, Gm13688, Abca14, B530045E10Rik, Fras1, Gm12159, Gm48488, 9030404E10Rik, Hmcn1, Igkv4-68, Wnt7a, Tmem74, Ighv1-66, Cr2, Slc5al1, Lrrc17, Slc22a14, Dipk2b, Fgf18, Ndp, Gm17334, Btn12, Gm10432, Colq, Gm48662, 4833412C05Rik, Gm12576, Igkv6-17, Gm13816, Tmem266, Ighv5-6, Cntn4, Rbp7, Insyn2a, Slc5a12, Gm9947, Igkv12-89, Gm26795, Ddx4, Dclk3, Hpcal4, Ndnf, Igkv17-121, Slurp1, Ano4, Ighv1-7, Rimklb, Igkv4-80, Cyp11b1, Cyp11a1, Akr1cl, Hsd3b1, Scg2, Th, Cyp21a1, Chga, Cyp21a2-ps, Pnmt, Chgb, Cyp11b2, Caln1, Kcnk9, Dbh, Gm28382, Pla2g2f, Nr5a1, Pcskln, Mgarp, 4930486L24Rik, Cryba4, Atp2b3, Resp18, E330017L17Rik, Pcsk1, Snap25, Rims4, Slc24a2, Phox2b, Slc18a2, Eef1a2, Chrna3, Hand2os1, Cntnap5a, Rasgrf1, Hand2, Pnmal1, Necab2, Tbx20, Stmn3, Gm17322, St8sia3, Rab33a, Synpr, Rit2, Camkv, Gdap1, Soga3, Mdga2, Mab2112, Nrsn1, Cadps, Gnb3, 1520401A03Rik, Nrg3, Spock1, Cdk5r2, Ina, Actl6b, Npy2r, Gal, Nrsn2, Gabrg2, Gpr158, Slc7a14, Sphkap, Tmem255a, 8430419K02Rik, Scg3, Gria1, Txndc2, Gm27477, Gria2, Gjd2, Lrrtm2, Hsd3b6, Diras1, Omg, Gm49391, Fam155a, Gm2007, Kcnq2, Gm42372, Is11, Lingo3, Prl8a2, Adh7, Lgi1, Gm47815, Gm14592, Cdx4, Marcksl1-ps4, Sstr3, Pcdh10, Gm27000, Tub, Mc2r, Il20ra, Grin2a, IL-1B, IL-10, Tnf-α, Ifn-γ, Nos1, Sele, Sell, Selp, Acat3, Tnfrsf1b, Csf1, Cx3cl1, and Ptgs2, Ccl12, Ccr2, Ccr5, Cxcl11, and/or Isg15.

Also disclosed herein are methods of reversing the expression profile of one or more genes encoding mediators of inflammation of any preceding aspect, wherein the expression of the one or more genes increases during microbial inflammation and wherein the one or more genes are selected from the group consisting of I110, Acod1, Saa3, Gpr31b, Fgf23, Igfbp1, Mrgpra2b, Ccl3, Ccl4, Gpr84, Adamts4, Madcam1, Olfm4, Il1f9, Gpr31a, Gpr31c, Ptx3, Slc7a11, Gm6553, Tmprss11g, Tac1, Il1f6, Ccl2, Psg16, Fosl1, Sprr2a2, Serpina3m, Il1rn, Ccl20, Tnf, Gm43181, Clec4e, Gm45774, Ugtla10, Kcnt1, Mt1, Mefv, Clec4d, Ms4a4a, Btbd16, Spic, Orm1, Serpina3f, Il1b, Serpina10, 9830107B12Rik, Gm49839, Msr1, Ankrd2, Saa4, S100a8, Hpx, Apoa4, Steap4, Cxcl10, S100a9, Wfdc17, Gnat1, Fgg, Fgb, Lilr4b, 2010003K11Rik, Serpina3n, AA467197, Gm16685, Timp1, Fga, Ifitm6, Lcn2, F10, Leap2, Retnlg, Ccl7, Ikbke, Vcan, F13a1, Serpina3g, 1200007C13Rik, Ngp, Siglece, Lrrc25, Gm10309, Trem1, Slc39a14, Fgl1, 1127, Tcp10b, Serpina3i, Cxc19, A730049H05Rik, Gm8883, Fpr2, Saa2, Saa1, Prok2, B230303A05Rik, Gm43079, Gm49383, Cldn14, Gm15056, U90926, Stfa2, Prokr2, Lbx1, Cstdc5, Tarm1, Mrgpra2a, Cstdc4, Gm48786, Gm19510, Gm32089, Gm5966, Stfa3, Tmem132e, Orm2, Gm28551, A630023A22Rik, Gm7457, Serpina7, Bmerb1, Klk9, 1700011B04Rik, Asprv1, Dmbt1, Tmprss11d, Slc5a7, Ifitm7, Gm38340, Pglyrp3, Gm26748, 4930431P03Rik, 4732490B19Rik, Sectm1b, Gm4948, Gm20406, Ras110b, Gm36079, Sult2a3, Apcs, Mymk, Nkx2-9, Gm26535, Rnase2a, Slc22a20, Stfa211, Gm4949, Gm11738, Gm29686, Serpine3, Gm807, Gm26444, Trdv5, Sultle1, Serpina3e-ps, 4930578G10Rik, Gm37645, Ceacam19, Ackr1, Ms4a3, B130046B21Rik, Ctcflos, Doc2b, Gm49573, Gm38275, Tex44, Gpr22, Ceacam10, E030018B13Rik, Gm37131, Htr3a, Slc13a5, Nxf3, Gm49427, Pde6h, Ubd, Fcrlb, Sult2a5, Tat, Gm6610, Gm44292, Gm44165, Prss22, Gm11714, Gm49368, Gm48065, Aoah, Tctex1d1, Phox2a, Slc36a3, Sult2a1, Gm37347, Gm50083, Epx, Gm26965, Reg3g, Cela3b, Gm12503, Gpbar1, Dnah6, Fut2, Muc4, Gm43189, Angpt17, Pcdh8, Sftpa1, Mlana, Gm16350, Syt2, Gm17055, Ppy, Ccdc33, Gm33280, Gm45643, Lrit3, Sbk2, Slc6a19os, Slamf1, Gm5541, Fgf21, Gm49392, Slc6a16, Olr1, Gm35028, Slfn4, Gm20548, Gm40709, Speer4f2, Gm13363, Egr2, Gm44260, Marc1, Crygn, Lpo, Gabrp, Bpifc, Fgf18, Lncenc1, Prss27, Muc19, Gm39929, Gm2670, 5830487J09Rik, Olfr1442, Krt20, Gm19684, Gm33424, 4732419C18Rik, Gm37621, Creb313, Gm14319, Dnah10, Areg, Armc12, Eif4ebp3, Gdf6, Gm49037, Gm42793, Derl3, Nptx2, Atp12a, Kcna7, Prg4, Ctla4, Gm44164, Mmp7, Gm44949, Slc17a8, Lamb3, Plekhs1, Trim17, Dusp8, 4930422C21Rik, Gm10727, Col27a1, Tacc2, Fat2, Lif, Klk1b1, Tsc22d1, Wnt8b, Grem1, Hesx1, Hhipl1, Ugtla5, Klk1b4, Gm8947, A530013C23Rik, Mylk4, Cyp2a5, Muc1, Gm44588, Gm45053, IL-1B, IL-10, Tnf-α, Ifn-γ, Nos1, Sele, Sell, Selp, Acat3, Tnfrsf1b, Csf1, Cx3cl1, and Ptgs2, Ccl12, Ccr2, Ccr5, Cxcl11, and/or Isg15.

In one aspect, disclosed herein are methods of reversing the expression profile of one or more genes encoding mediators of inflammation of any preceding aspect, wherein the expression of the one or more genes decreases during microbial inflammation and wherein the one or more genes are selected from the group consisting of Ano5, Gsg1l, Spon2, Oxtr, Rnf112, Ppp2r2c, Crabp1, St6galnac3, Agtr2, Gjc2, Gm33195, Gabra4, Aplnr, E030013I19Rik, Pcdh12, Chp2, Gng8, Pla2g2d, Gm20683, Mansc4, Gm10824, Ces2b, Kank4, Zmat4, Cdr1os, Wnt2, Gm17040, Wfdc16, Rab42, Tbxa2r, Plppr5, Misp3, Mmp28, Lrrn4, Sost, Tmem200a, Shisa2, Stmn2, Chrm1, Tmeff2, 2810433D01Rik, Pabpc41, Dact3, Mett124, Gpr34, D430019H16Rik, Hoxc5, Tekt5, Megf10, Wscd1, Gm32219, Tril, Agtr1b, 2610203C22Rik, Abcg3, Star, Grik2, Grin2c, Vstm5, Smad9, Pianp, Rnf144a, Cthrc1, Nxpe4, Mrc2, Cx3cr1, 115ra, Kcnc4, Fam163a, Cxcr3, A330074K22Rik, Gm11627, Mmp11, Fam181b, Cd209b, Tnfrsf19, Sept4, Nlgn3, Baalc, Ngb, Nxph3, Klb, Igkv4-72, Ptprv, Nrep, Itga8, Myct1, Palm, Syt3, Tspan18, Piezo2, Nat8f3, Cngb1, Cd209g, Faim2, Gfra2, Srpk3, Gm3716, Klhl13, Mogat2, Tmem233, Duxb13, Rnase1, Defb42, AL935121.1, Zp1, Snurf, Gm25541, 4933403008Rik, 1810046K07Rik, Gm5639, Myh2, Gm765, Gm50242, Gm6018, Ackr4, Tnfrsf17, Gm37858, Igkv14-130, Ptprh, Gpr165, Gm47652, Gm16534, 0610012D04Rik, Gm36569, Gzmk, Gm9918, Gm33677, St6gal2, Krtap17-1, Olfr1342, Lhcgr, Ccdc42, Zfp985, Igkv4-70, Gm31517, Gm9767, Olfr71, Slc26a3, Trpv3, Gm49727, Gm43951, E330018M18Rik, Calcb, Rspo2, Glp1r, Fam124b, Amtn, 4930570G19Rik, Gm11574, Pla2g2e, Sfrp2, Pigz, Gm13688, Abca14, B530045E10Rik, Fras1, Gm12159, Gm48488, 9030404E10Rik, Hmcn1, Igkv4-68, Wnt7a, Tmem74, Ighv1-66, Cr2, Slc5a11, Lrrc17, Slc22a14, Dipk2b, Fgf18, Ndp, Gm17334, Btn12, Gm10432, Colq, Gm48662, 4833412C05Rik, Gm12576, Igkv6-17, Gm13816, Tmem266, Ighv5-6, Cntn4, Rbp7, Insyn2a, Slc5a12, Gm9947, Igkv12-89, Gm26795, Ddx4, Dclk3, Hpcal4, Ndnf, Igkv17-121, Slurp1, Ano4, Ighv1-7, Rimklb, Igkv4-80, Cyp11b1, Cyp11a1, Akr1c1, Hsd3b1, Scg2, Th, Cyp21a1, Chga, Cyp21a2-ps, Pnmt, Chgb, Cyp11b2, Caln1, Kcnk9, Dbh, Gm28382, Pla2g2f, Nr5a1, Pcskln, Mgarp, 4930486L24Rik, Cryba4, Atp2b3, Resp18, E330017L17Rik, Pcsk1, Snap25, Rims4, Slc24a2, Phox2b, Slc18a2, Eef1a2, Chrna3, Hand2os1, Cntnap5a, Rasgrf1, Hand2, Pnmal1, Necab2, Tbx20, Stmn3, Gm17322, St8sia3, Rab33a, Synpr, Rit2, Camkv, Gdap1, Soga3, Mdga2, Mab2112, Nrsn1, Cadps, Gnb3, 1520401A03Rik, Nrg3, Spock1, Cdk5r2, Ina, Act16b, Npy2r, Gal, Nrsn2, Gabrg2, Gpr158, Slc7a14, Sphkap, Tmem255a, 8430419K02Rik, Scg3, Gria1, Txndc2, Gm27477, Gria2, Gjd2, Lrrtm2, Hsd3b6, Diras1, Omg, Gm49391, Fam155a, Gm2007, Kcnq2, Gm42372, Isl1, Lingo3, Prl8a2, Adh7, Lgi1, Gm47815, Gm14592, Cdx4, Marcksl1-ps4, Sstr3, Pcdh10, Gm27000, Tub, Mc2r, I120ra, and/or Grin2a.

Also disclosed herein are methods of reversing the expression profile of one or more genes encoding mediators of inflammation of any preceding aspect, wherein the microbial inflammation is acute inflammation, subacute inflammation, chronic inflammation, organ-specific inflammation, systemic inflammation, sepsis, or polymicrobial sepsis.

In one aspect, disclosed herein are methods of reversing the expression profile of one or more genes encoding mediators of inflammation of any preceding aspect, wherein the microbial inflammation is localized in the blood, brain, sinuses, upper respiratory tract, or lungs, heart, bone marrow, spleen, liver, kidneys, genito-urinary tract, bladder, aural cavities, stomach, intestines, skin, eyes, teeth, and/or gingiva.

Also disclosed herein are methods of reversing the expression profile of one or more genes encoding mediators of inflammation of any preceding aspect, wherein the microbial inflammation is caused by a viral infection caused by Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2.

In one aspect, disclosed herein are methods of reversing the expression profile of one or more genes encoding mediators of inflammation of any preceding aspect, wherein the microbial inflammation is caused by a bacterial infection caused by Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, or other Yersinia species. In some aspects, the bacteria causing the bacterial infection is not Bacillus anthracis.

Also disclosed herein are methods of reversing the expression profile of one or more genes encoding mediators of inflammation of any preceding aspect, wherein the microbial inflammation is caused by a fungal infection caused by a fungi including, but not limited to Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

Also disclosed herein are methods of reversing the expression profile of one or more genes encoding mediators of inflammation of any preceding aspect, wherein the microbial inflammation is caused by a parasitic infection including, but not limited to an infection of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

In one aspect, disclosed herein are methods of reducing, decreasing, removing, clearing, and/or eliminating a microbial infection (such as, for example, a viral, bacterial, fungal, or parasitic infection) and/or the microbial burden of the microbial infection in the blood, brain, sinuses, upper respiratory tract, or lungs, heart, bone marrow, spleen, liver, kidneys, genito-urinary tract, bladder, aural cavities, stomach, intestines, skin, eyes, teeth, or gingiva of a subject comprising administering to the subject a therapeutically effective amount of a composition comprising an NTCI (such as, for example, SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41). In some aspects, the method can further comprise the administration of an anti-microbial agent.

Also disclosed herein are methods of reducing, decreasing, removing, clearing, and/or eliminating a microbial infection and/or the microbial burden of the microbial infection of any preceding aspect, wherein the microbial burden is caused by a viral infection caused by Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In some aspects, disclosed herein are methods of reducing, decreasing, removing, clearing, and/or eliminating a microbial infection and/or the microbial burden of the microbial infection of any preceding aspect, wherein the microbial burden is caused by a bacterial infection caused by an infection with a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species. In some aspects, the bacteria causing the bacterial infection is not Bacillus anthracis.

Also disclosed herein are methods of reducing, decreasing, removing, clearing, and/or eliminating a microbial infection and/or the microbial burden of the microbial infection of any preceding aspect, wherein the microbial burden is caused by a fungal infection; wherein the fungal infection is an infection with a fungi including, but not limited to Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

In one aspect, disclosed herein are methods of reducing, decreasing, removing, clearing, and/or eliminating a microbial infection and/or the microbial burden of the microbial infection of any preceding aspect, wherein the microbial burden is caused by a parasitic infection; and wherein the parasitic infection is an infection with a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

### BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, and 1C show the 8 Genes Encoding Mediators of Innate Immunity and Inflammation Were Selected for the Comparison of the Two Methods of Genomic Analysis of the Lungs in Sepsis. The selected genes display strong response to sepsis as detected by the NGS (1A) and verified by real-time quantitative reverse transcription PCR (1B). The relative levels of expression in qRT PCR (1B) were established using Livak's methods (2^{-ΔΔCt}) with the 18S rRNA gene as a reference and the Mock Control (unchallenged and not treated mice) group as the calibrator (control). Data is presented as a mean+S.E.M. (n=3 for Mock Control, n=5 for all other conditions). FIG. 1C shows the level of cytokines IL-6, IL-10, TNF-α, and IFN-γ was determined in blood plasma collected postmortem from hearts of mice euthanized 6 h after CM (or Sham) challenge (groups Sham+Saline, CM+Saline, and CM+cSN50.1) or 1 hr. after last NTCI treatment (cSN50.1 only). Data is presented as a mean±SEM (n=5). The selective nuclear blockade with the NTCI suppressed expression of 7 analyzed genes while the gene encoding IL-6 remained increased after the NTCI treatment, as determined by the two methods of genomic analysis and confirmed by proteomic analysis in blood plasma. A statistical analysis in Panels B and C was performed using an ordinary one-way ANOVA with an uncorrected Fisher's LSD test for a multiple comparison, *p<0.05, **p<0.005, ***p<0.0005, ****p<0.0001. The significance levels displayed over the error bars of CM+Saline and CM+cSN50.1 columns represent the statistical difference of each compared to Sham+Saline samples.

FIG. 2 shows similar Expression of Genes Encoding Cytokines, Chemokines and Signaling Intermediates in the Lungs and Kidneys, and their Less Robust Decrease in the Kidneys of the NTCI-Treated Animals. The NGS analysis of total RNA samples extracted from lungs and kidneys of CM-challenged mice treated with the NTCI for 6 hours (n=3, see Materials and Methods for details). Please note that elevated genes in the lungs were particularly susceptible to the nuclear blockade with the NTCI indicating its utility for treatment of Acute Respiratory Distress Syndrome, a life-threatening complication of sepsis caused by viruses and bacteria.

FIGS. 3A, 3B, 3C, and 3D show the genomic Response to Polymicrobial Sepsis in the Lungs and Kidneys. FIG. 3A shows the response of lungs and kidneys' genome to sepsis. Red—genes with increased expression, blue—genes with decreased expression, yellow—genes with unchanged expression. Please note that the pie charts are constructed based on genes significantly expressed (P_{adj}<0.05). The 1,897 genes displayed similar pattern of expression in both organs. These genes are termed “common” or “shared” or organ-non-specific genes. FIG. 3B shows the overall view of the gene expression in the lungs and kidneys of mice following the Selective Nuclear Blockade with cell-penetrating NTCI (cSN50.1 peptide). FIG. 3C shows the impact of NTCI treatment on gene expression in control animals without sepsis. FIG. 3D shows a detailed analysis of the Selective Nuclear Blockade of the increased, decreased or unchanged pools of genes in sepsis as presented in FIG. 3A.

FIG. 4 shows selective Nuclear Blockade with the NTCI Significantly Reduced Bacterial Dissemination in Blood, Lungs, Spleen, and Kidneys. Mice with induced polymicrobial peritonitis were treated for 12 hours with the NTCI without antimicrobial therapy. Blood, lungs, spleen, and kidneys were collected and processed for bacterial count (n=5). Bacterial dissemination in blood and three major organs was analyzed by nonparametric t test with Mann-Whitney rank comparison. The data is presented as a mean±SEM. ****p<0.0001, ns—not significant. Importantly, the 9-fold increase in microbial clearance in the spleen is clinically relevant. Patients with hyposplenism due to celiac disease or following bone marrow transplantation are at a significant risk of fulminant sepsis

FIG. 5 shows a cartoon displaying sepsis-Causing Microbial Agents Sensed by Toll-Like Receptors and Other Extracellular and Intracellular Detectors Activate Multiple Transcription Factors in Immune and Non-Immune Cells (e.g., Endothelial and Epithelial Cells). Signals generated by these receptors/sensors/channels activate at least 7 families of stress-responsive transcription factors (SRTFs) and metabolic transcription factors (MTFs) Please note that the AP1 complex comprises two distinct transcription factors, cFos and cJun. Transcription factors are ferried to the inflammatory and/or metabolic regulomes in the nucleus by nuclear transport shuttles (importin α5 and importin β1) that are selectively controlled by the Nuclear Transport Checkpoint Inhibitors (NTCIs). Thus, selective nuclear blockade shields the inflammatory and metabolic regulomes in the nucleus thereby reducing the activation of a myriad (in thousands) of genes responding to sepsis. NPC: Nuclear Pore Complex.

FIG. 6 shows determination of Cecal Microbiome Lethal Dose Causing 50% Mortality. 8-week-old mice were challenged with a single i.p. injection of CM at a dose corresponding to the indicated CFUs. Mice were observed for 7 days after the CM-challenge without any treatment and the survival was recorded. Original observation is presented as the Kaplan-Meier survival plot (left panel). A dose-dependent survival represents a linear correlation according to the following equation: Survival=−1.73×10⁴×CFU+220.1 with R²=0.9875 (p=0.0072). The LD₅₀ corresponds to 9.85×10⁵ CFU/kg of body mass.

FIG. 7 shows signaling to the Nucleus by Inflammatory and Metabolic Transcriptional Pathways. In response to multiple proinflammatory cues outside of cell, the six selected transcriptional cascades, depicted schematically in this figure, are activated to trigger signaling to the genome in the cell's nucleus. This signaling is dependent on multiple receptors and their adapters, and other signal transducers (kinases, phosphatases, ubiquitin modifiers, and nuclear import adaptors) that form the signalosomes and other multimeric complexes. Proinflammatory Stress-Responsive Transcription Factors (SRTFs) encompass NF-KB, AP-1 (cFos, cJun), NFAT, and STAT1, among others. Metabolic Transcription Factors (MTFs) comprise Sterol Regulatory Element Binding Proteins (SREBPs) and Carbohydrate Regulatory Element Binding Proteins (ChREBPs), among others. Depicted SRTFs and MTFs converge at the Nuclear Transport Checkpoint “staffed” by Importin α5 (Imp α5) and importin β1 (Imp β1), among others. Imp α5 recognizes the Nuclear Localization Sequence (NLS) displayed in depicted SRTFs while Imp β1 recognizes the basic Helix-Loop-Helix leucine-rich zipper (bHLH-Zip) domain displayed in the SREBPs and ChREBPs. Moreover, Imp β1 recognizes its cognate binding site (importin beta binding domain) on Imp α5 ferrying its attached “payload” (i.e. transcription factor) to guide it to the Nuclear Pore Complex (NPC), and the genome. A nuclear form of SREBPs (nSREBPs) is ferried to the cell's nucleus solely by the Imp β1, which recognizes SREBP's N-terminal motif, bHLH-Zip. Other metabolic transcription factors, ChREBPs, display both bHLH-Zip and NLS. Once in the nucleus, SRTFs and MTFs bind to their cognate transcription factor binding sites in the gene promoters and enhancers to initiate transcription of the multiple genes that encode mediators of inflammatory and metabolic pathways. Some of these inflammatory and metabolic genes are displayed in the nucleus. The interaction between “activated” or “processed” TFs and the nuclear transport shuttles can be inhibited by the cell-penetrating peptides termed Nuclear Transport Checkpoint Inhibitors aka. Nuclear Transport Modifiers (NTMs). Hence, the NTCIs aka NTMs dismantle the nuclear transport checkpoint by competitive binding to Imp α5 or Imp β1 thereby silencing the inflammatory and metabolic regulomes in the genome.

FIG. 8 shows distribution of the log₂(FC) values. The Log₂(FC) values were rounded down to the nearest decimal point (0.1), and the equal values were added up. Data points were plotted against the log₂(FC) to determine the center of distribution and the standard deviation used for selection of genes with increased, decreased, and unchanged expression.

FIGS. 9A and 9B show the determination of Potential Direct Antibacterial Action of the NTCI. FIG. 9A shows the potential NTCI's antibacterial action toward the constituents of the cecal microbiome (CM) was determined by the Kirby-Bauer's Antimicrobial Susceptibility Test (AST) with meropenem as a positive control. Sterile discs were loaded with the indicated amount of NTCI and placed on TSA+5% sheep blood agar plates before application of the CM. The plates were incubated overnight at 37° C. The Kirby-Bauer's AST indicates no effect of the NTCI on bacterial growth. FIG. 9B shows the test of NTCI diffusion to the agar. The disc saturated with the FITC-labeled NTCI was placed on regular agar plate without CM, incubated for 12 hrs. at 37° C., and visualized by the fluorescent imaging (left panel). To improve image quality by reducing strong signal emanating from non-diffused FITC-NTCI, which was accumulated on the disc and underneath, the disc and corresponding portion of the agar were removed from the plate (right panel). Please note that the portion of the agar gel was also removed from across the plate to avoid FITC-NTCI diffusion. The FITC-free area (right side of the plate) was then used as a basis for determining the background fluorescence.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS OF TREATING SEPSIS

Although compositions, kits, cells, and methods similar or equivalent to those described herein can be used in the practice or testing of the present invention, suitable compositions, kits, cells, and methods are described below. All publications, patent applications, and patents mentioned herein are incorporated by reference in their entirety. U.S. patents applications Ser. Nos. 14/349,918, and U.S. Pat. No. 7,553,929, for example, are incorporated by reference in their entireties. In the case of conflict, the present specification, including definitions, will control. The particular embodiments discussed below are illustrative only and not intended to be limiting.

The response to the sepsis-causing infections begins when the pattern-recognition receptors, Toll-like Receptors (TLRs), detect pathogen-associated molecular patterns, such as endotoxic lipopolysaccharide (LPS), the constituent of Gram-negative bacteria, the cause of two-thirds of sepsis cases. Intracellular NOD-like receptor family senses conserved bacterial peptidoglycans. The innate immunity receptors, e.g., TLRs, are displayed in immune and non-immune cells, including microvascular endothelial cells and epithelial cells. Therein, they activate nuclear signaling pathways that mobilize families of the stress-responsive transcription factors, SRTFs (e.g., NF-κB, cFos, cJun, STAT1 and STAT3, NFAT, and Nrf2), and Metabolic Transcription Factors, MTFs (SREBPs and ChREBPs). Their access to the inflammatory and metabolic regulomes in the cell's nucleus is controlled by the nuclear transport checkpoint comprising Importin α5 and Importin β1, among others. Importin α5, aka karyopherin α1, is one of the six members of the importin α family. Importin α5 recognizes Nuclear Localization Sequence (NLS) on multiple proinflammatory SRTFs and metabolic transactivators, ChREBPs. Importin β1 selectively recognizes SREBPs. These proinflammatory and metabolic transcription factors activate a myriad of genes that encode mediators of microbial and metabolic inflammation.

Life-threatening sepsis progression is linked to the endothelial cells' activation and subsequent injury in the small blood vessels (<100 μM diameter) supplying all organs. Microvascular endothelial activation and injury promote extravasation of leukocytes and lymphocytes and leakiness of small blood vessels. They contribute to the acute respiratory distress syndrome (ARDS) and the acute kidney injury (AKI). These failing organs comprise the major therapeutic challenges in sepsis. In turn, Septic Shock, manifested by hypotension that is nonresponsive to fluid resuscitation or vasopressors, culminates sepsis. Until now, the genomic response to sepsis in the major organs other than blood has not been reported.

Herein, we submit that the expression of thousands of genes in the lungs (prone to ARDS) and kidneys (succumbing to AKI) was increased or decreased in experimental sepsis evolving from polymicrobial peritonitis. We also found that this genomic response to polymicrobial sepsis was countered by the selective nuclear blockade with the Nuclear Transport Checkpoint Inhibitor (NTCI), aka Nuclear Transport Modifier (NTM). Short (6 hours) treatment with the NTCI changed the expression of a multitude of organ-specific and shared genes in the lungs and kidneys responding to the experimental sepsis. Since humans and mice show on average 85% similarity of their DNA sequences that code for proteins (National Human Genome Research Institute), our data provide relevant evidence that the major organs-based genomic response to sepsis is an important mechanism in its initiation, progression, and resolution.

The NTCIs are cell-penetrating anti-inflammatory and anti-metabolic peptides rapidly reaching immune cells in the blood and major organs e.g., lungs. Moreover, they enter non-immune cells (e.g., endothelial cells lining blood vessels), as well as other cells (e.g., epithelial cells) in the major organs, attacked by sepsis-causing microbial agents. The NTCIs cross the cell membrane of immune and non-immune cells through the phospholipid bilayer without involving the chirally-specific receptor or transporter. Importantly, the NTCIs bypass the endosomal compartment thereby avoiding intracellular degradation by lysosomal proteases. In the cytoplasm, the NTCIs simultaneously target the two nuclear import adaptor proteins, importin α5 and importin β1 required for the nuclear translocation of proinflammatory SRTFs and MTFs. Thus, the NTCIs impede the nuclear entry of the key transcription factors, SRTFs and MTFs, thereby preventing their activation of the inflammatory and metabolic regulomes, respectively.

Astoundingly, we found that the selective nuclear blockade by NTCI resulted in reprogramming of 3,735 sepsis-induced genes in the lungs and 1,951 sepsis-induced genes in the kidneys. Moreover, the NTCI increased microbial clearance by reducing the sepsis-causing bacterial dissemination 18-fold in the blood, 9-fold in the spleen, and 11-fold in the lungs after the 12-hour treatment.

Small transcription factors (<45 kD), usually those regulating the housekeeping genes that encode cell survival factors, have free passage from the cytoplasm to the nucleus. In contrast, nuclear transport of transcription factors larger than 45 kD, such as SRTFs, is guided by one or more nuclear localization sequences (NLSs). These intracellular “zip codes” are displayed on SRTFs upon stimulation of immune and non-immune cells by microbial insults. NLSs are then recognized by nuclear transport adaptor proteins, importins/karyopherins alpha (Imp α). The stimulus-induced formation of SRTF and importins α complexes also encompasses importin beta 1 (Imp β1), which is recognized by nuclear pore proteins to allow translocation of the cargo to the nucleus. Until recently, nuclear transport has been targeted through the forced expression of genes that encode inhibitors of proinflammatory SRTFs, such as the degradation-resistant inhibitor of NF-κB termed IκBα. However, NF-κB is only one of multiple SRTFs that mediate signaling to the nucleus in response to infection. Other SRTFs, such as AP-1, STAT1 and NFAT, are also transported to the nucleus during the inflammatory response yet their nuclear transport is not impeded by IκBα; contrarily, the AP-1 pathway is activated. Targeting nuclear transport, a pivotal checkpoint integrating translocation of multiple transcription factors to the nucleus, can be a more efficient strategy than targeting signaling pathways of individual transcription factors. This concept was proven by design and development of NTCIs.

NTCIs target the nuclear transport shuttles, Imp α5 and Imp β1, that translocate SRTFs to the nucleus and control signal transduction pathways, which culminate in genomic reprogramming. NTCIs modulate signaling to the nucleus mediated by transcription factors that include but are not limited to NFκB, AP-1, NFAT, STAT1 that utilize importins alpha and beta heterodimer, or SREBP1a, SREBP1c, and SREBP2, that utilize solely importin beta for nuclear transport whereas ChREBP can utilize primarily importins alpha/beta heterodimer for nuclear translocation. SRTFs such as NFκB, AP-1, NFAT, STAT1 are transported to the nucleus in response to proinflammatory stimuli. In the nucleus, SRTFs activate genes that encode mediators of inflammation. Examples of NTCIs include SN50, cSN50 and cSN50.1 described in more detail in the following paragraphs, as well as the sequences set forth in Table 7.

SN50, cSN50, cSN50.1 and their derivatives inhibit nuclear import of SRTFs by binding to their target, importin alpha 5 (Imp α5) known also as karyopherin α1 (KPNA1, SRP1). Adaptor proteins targeted by these peptides constitute a recognition arm of nuclear import machinery. They recognize nuclear localization sequence (NLS) on SRTFs, ChREBPs or other transcription factors, e.g. NRF2, and carry this cargo across the nuclear pore. Imp α5 (KPNA1/SRP1) forms a complex with importin β1 (Imp β1) which docks at the cytoplasmic rim of the nuclear pore.

SN50, cSN50, and cSN50.1 and their derivatives also bind Imp β1 and compete with its transporting of SREBP 1 and 2 to the nucleus. As these transcription factors do not display NLS recognized by importins α, their transport is independent of Imp α5. The latter recognizes NLS motif on SRTFs and ChREBPs.

In recent preclinical studies, a highly soluble cell-penetrating NTCI (cSN50.1), with dual specificity was used. This NTCI has segments that bind both Imp α5, which recognizes NLS derived from NFκB1, and Imp β1, which recognizes the signal-sequence hydrophobic region (SSHR) derived from Fibroblast Growth Factor 4. SSHR also serves as a membrane translocating motif (MTM) to enable intracellular delivery of peptides and proteins through an ATP- and endocytosis-independent mechanism. This and other NTCIs have been shown to inhibit nuclear translocation of SRTFs and metabolic transcription factors, Sterol Regulatory Element-Binding Proteins (SREBPs) and Carbohydrate-Responsive Element-Binding Proteins (ChREBPs) thereby reducing inflammatory responses, microvascular injury, apoptosis and hemorrhagic necrosis as well as correcting metabolic derangements (eg. hyperlipidemia, with a concomitant gain in survival, in models of lethal shock induced by bacterial toxins.

A novel form of immunotherapy that targets nuclear import as described herein can arrest inflammation-driven destruction of microbe-infected tissue and surrounding area of a given organ. With respect to microbial inflammation (such as, for example, acute inflammation, subacute inflammation, chronic inflammation, skin-specific inflammation, systemic inflammation), pro-inflammatory signaling initiated through stimulation of the principal receptors of innate immunity, Toll-like receptors (TLRs), is one mechanism that activates antigen-presenting cells (APCs). Reprograming of gene regulatory networks in response to a multitude of microbial insults is dependent on signaling to the host cell's nucleus comprising a fundamental process of microbial inflammation. Inhibiting nuclear transport at a common “checkpoint” located downstream of TLRs and cytokine receptors globally suppresses expression of inflammatory genes thereby calming the genomic storm and averting multiple organ injury.

It is understood and herein contemplated that by reducing the levels of SRTF and metabolic transcription factors, ChREBPS, and SREBPs, in a cell's nucleus, the disclosed NTCI can reverse or inhibit the genomic reprogramming associated with microbial inflammation (such as, for example, acute inflammation, subacute inflammation, chronic inflammation, organ-specific inflammation, systemic inflammation, and/or end stage of microbial inflammation, sepsis including, but not limited to polymicrobial sepsis). Accordingly, described herein are methods of reversing the expression profile (i.e., decreasing, reducing, or inhibiting expression when the expression profile increases or increasing expression when the expression profile decreases) of one or more genes encoding mediators of inflammation in a subject whose expression modulates during microbial inflammation as a result of microbial infection (such as, for example, a viral, bacterial, fungal, or parasitic infection) comprising administering to the subject a therapeutically effective amount of a nuclear transport checkpoint inhibitor (NTCI).

Administration of the NTCI composition decreases inflammation by attenuating expression profile change of at least one stress-responsive transcription factor-regulated gene, or at least one ChREBPs and/or one SREBPs-regulated gene. Thus, the effective dose is an amount effective for reducing importin alpha-mediated nuclear translocation of at least one stress response SRTF or one ChREBPs and can ultimately provide for/facilitate the clearance of the infecting microbe without the further requirement of an antimicrobial agent.

An important aspect of the NTCI exemplified by cSN50.1 peptide and its congeners is their ability to reach the site of infection and the infected host cell in the skin, as well as other myeloid, lymphoid, and non-lymphoid organs. The mechanism of intracellular delivery of this class of cell-penetrating peptides has been elucidated and an endocytosis-independent process of crossing the plasma membrane mediated by the membrane-translocating motif (MTM), which is based on the SSHR derived from Kaposi FGF, has been. The amphipathic helix-based structure of SSHR facilitates its insertion directly into the plasma membrane and the tilted transmembrane orientation permits the translocation of the NTCI through the phospholipid bilayer of the plasma membrane directly to the interior of the cell without perturbing membrane integrity. This mechanism explains the efficient delivery of SSHR-guided cargo across the plasma membrane of multiple cell types involved in microbial inflammation, autoimmune inflammation, allergic inflammation, metabolic inflammation, neoplastic inflammation, and physical inflammation.

The NTCIs disclosed herein are derived from N50-containing NTCIs (SN50, cSN50, and cSN50.1) that are comprised of a hydrophilic N50 motif patterned on the nuclear localization sequence (NLS) region of the NFκB1/p50 subunit (Table 7) fused to a motif from the signal sequence hydrophobic region (SSHR) of human fibroblast growth factor 4. The SSHR allows peptides to cross the plasma membrane by an ATP- and endosome-independent mechanism thereby avoiding intracellular degradation by lysosomal proteases, and the N50 motif was designed to bind to importins α during stimulus-initiated signaling and thereby limit docking of NLS-bearing SRTFs to their adaptor proteins and reduce nuclear import of activated STRFs. Any mimetics, derivatives, or homologs of SN50, cSN50, and cSN50.1 may be used in the compositions, methods, and kits disclosed herein.

TABLE 7
Amino Acid Sequences of Peptides Used
Peptide Sequences

| NTCI | SSHR | NLS | SEQ ID NO: |
|---|---|---|---|
| N50 | | VQRKRQKLMP | 10 |
| N50M | | VQRDEQKLMP | 11 |
| cN50.1 | | CVQRKRQKLMPC | 12 |
| SN50 | AAVALLPAVLLALLAP | VQRKRQKLMP | 13 |
| SSHR-1 | AAVALLP | | 14 |
| SSHR-2 | AVLLALLAP | | 15 |

N50—sequence derived from the NLS region of NFκB1/p50; N50M—sequence of control peptide with KR to DE mutation (bolded); cN50.1—sequence of cyclized version of N50 just as cSN50.1 is a cyclized version of SN50. Hydrophobic regions of the SSHR domain are distinguished from the cluster of basic amino acids (NLS). NTCI indicates nuclear transport modifier; SSHR, signal sequence hydrophobic region; NLS, nuclear localization sequence.

SN50 is a fragment linked peptide combining the SSHR of the Kaposi fibroblast growth factor (K-FGF) and the NLS of the p50 subunit of NFκB1. Any mimetics, derivatives, or homologs of SN50 may be used in the compositions, methods, and kits disclosed herein. The sequence of SN50 is AAVALLPAVLLALLAPVQRKROKLMP (SEQ ID NO: 13). Generation and use of SN50 is described in U.S. Pat. No. 7,553,929.

cSN50 is a fragment-designed cyclic peptide combining the hydrophobic region of the Kaposi fibroblast growth factor signal sequence with the nuclear localization signal (NLS) of the p50-NFKB1 and inserting a cysteine on each side of the NLS to form an intrachain disulfide bond. The amino acid sequence of cSN50 is AAVALLPAVLLALLAPCYVQRKRQKLMPC (SEQ ID NO: 1). Any mimetics, derivatives, or homologs of cSN50 may be used in the compositions, methods, and kits disclosed herein. Methods of making and using cSN50 are described, for example, in U.S. Pat. Nos. 7,553,929 and 6,495,518. These patents are incorporated herein by reference in their entireties.

cSN50.1 is a cyclized peptide having the sequence of cSN50 with the exception that the tyrosine at position 18 of cSN50, adjacent to the first cysteine, has been removed. Methods of making and using cSN50 are described, for example, in U.S. Pat. Nos. 7,553,929 and 6,495,518. The amino acid sequence of cSN50.1 is AAVALLPAVLLALLAPCVQRKRQKLMPC (SEQ ID NO: 2). The tyrosine at position 18 was removed from the sequence of cSN50 to increase solubility. cSN50 is soluble at levels of ranging from 2.0 mg/mL to 40 mg/mL depending on the method of synthesis and purification whereas cSN50.1 is soluble at levels of at least 100 mg/ml. Any mimetics, derivatives, or homologs of cSN50.1 may be used in the compositions, methods, and kits disclosed herein. cSN50.1 is also encompassed by SEQ ID NO: 3, SEQ ID NO: 4 and SEQ ID NO: 5. Additional examples of NTCIs include fragment-designed and synthesized peptides in which cargo is incorporated as two, rather than one, modules or cargos derived from intracellular proteins other than NFκB 1. Such additional examples include the sequences of SEQ ID NO: 6, SEQ ID NO: 7, SEQ ID NO: 8, and SEQ ID NO: 9.

Accordingly, the NTCI for use in the disclosed methods of treating, inhibiting, reducing, and/or preventing inflammatory skin diseases including, but not limited to microbial disease, autoimmune disease, autoinflammatory disease, metabolic disorder, neoplastic disorder, or physical injuries that are mediated by inflammation may be, for example, an NTCI having the sequence Xaa Xaa Xaa Xaa Leu Leu Pro Xaa Xaa Leu Leu Ala Leu Leu Ala Pro Xaa Xaa Xaa Gln Arg Lys Arg Gln Lys Xaa Xaa Xaa Xaa (SEQ ID NO: 3), wherein Xaa is any amino acid or is absent. For example, the Nuclear Transport Modifier can have the sequence Xaa Xaa Xaa Xaa Leu Leu Pro Xaa Xaa Leu Leu Ala Leu Leu Ala Pro Cys Xaa Xaa Gln Arg Lys Arg Gln Lys Xaa Xaa Xaa Cys, where Xaa is any amino acid or is absent (SEQ ID NO: 4). As another example, the Nuclear Transport Modifier can have the sequence Xaa Xaa Xaa Xaa Leu Leu Pro Xaa Xaa Leu Leu Ala Leu Leu Ala Pro Cys Xaa Gln Arg Lys Arg Gln Lys Xaa Xaa Xaa Cys, where Xaa is any amino acid or is absent (SEQ ID NO: 5). In one embodiment, the Nuclear Transport Modifier is cSN50.1 having the sequence set forth in SEQ ID NO: 2. In another example of an NTCI, the NTCI has the sequence Xaa Xaa Xaa Xaa Leu Leu Pro Xaa Xaa Leu Leu Ala Val Leu Ala Pro Xaa Xaa Xaa Gln Arg Lys Arg Gln Lys Xaa Xaa Xaa Xaa, where Xaa is any amino acid or is absent (SEQ ID NO: 6). In yet another example, the NTCI has the sequence Ala Ala Val Ala Leu Leu Pro Ala Val Leu Leu Ala Val Leu Ala Pro Cys Val Gln Arg Lys Arg Gln Lys Leu Met Pro Cys (SEQ ID NO: 7). In a further example, the NTCI has the sequence Xaa Xaa Xaa Xaa Leu Leu Pro Xaa Xaa Leu Leu Ala Val Leu Ala Pro Xaa Xaa Xaa Gln Arg Asp Glu Gln Lys Xaa Xaa Xaa Xaa, where Xaa is any amino acid or is absent (SEQ ID NO: 8). In another example, the NTCI has the sequence Ala Ala Val Ala Leu Leu Pro Ala Val Leu Leu Ala Val Leu Ala Pro Cys Val Gln Arg Asp Glu Gln Lys Leu Met Pro Cys (SEQ ID NO: 9). In one aspect the NTCI can be cSN50.1 beta comprising the amino acid sequence AAVALLPAVLLALLAPCVQRDEQKLMPC (SEQ ID NO:16). cSN50.1 beta is a cyclized peptide having the sequence of cSN50.1 with the exception that the lysine at the position 21 has been replaced by aspartic acid and the arginine residue at the position of 22 has been replaced by glutamic acid.

As mentioned above, NTCIs modulate signaling to the nucleus mediated by transcription factors that include but are not limited to NFκB, AP-1 (cFOS/cJUN), NFAT, STAT1, and STAT3 that utilize importins alpha5 and beta1 heterodimer, SREBP1a, SREBP1c, and SREBP2, that utilize solely importin beta for nuclear transport whereas ChREBP can utilize primarily importins alpha5 for nuclear translocation. In this example, the importin beta-selective NTCI reduces nuclear translocation of the nuclear forms of SREBP1a, SREBP1c, and SREBP2. Any suitable importin beta1-selective NTCI may be used. Examples of importin beta-selective NTCIs include but are not limited to peptide sequences that include an SSHR domain listed in Table 8 below and a cargo listed in Table 7 above. One example of such an importin beta-selective NTCI is AAVALLPAVLLALLAPVQRDEQKLMP (SEQ ID NO: 40) (i.e., a peptide sequence having the SSHR domain of AAVALLPAVLLALLAP (SEQ ID NO: 17) and the cargo of VQRDEQKLMP (SEQ ID NO: 11) as listed in Table 8 below). Additional examples of peptides designed to inhibit interaction of importin alpha with importin beta necessary for the formation of their heterodimer include AAVALLPAVLLALLAPRRRRIEVNVELRKAKK (SEQ ID NO: 18) (referred to as SIBB in Table 8), AAVALLPAVLLALLAPRRRRIEVNVELRKAKKDD (SEQ ID NO: 19) (referred to as SI-1 in Table 8). AAVALLPAVLLALLAPRRQRNEVVVELRKNKRDE (SEQ ID NO: 20) (referred to as SI-3 in Table 8), AAVALLPAVLLALLAPRRHRNEVTVELRKNKRDE (SEQ ID NO: 21) (referred to as SI-4 in Table 8), AAVALLPAVLLALLAPRRRREEEGLQLRKQKREE (SEQ ID NO: 22) (referred to as SI-5 in Table 8), AAVALLPAVLLALLAPRRRREEEGIQLRKQKREQ (SEQ ID NO: 23) (referred to as SI-7 in Table 8) and AAVALLPAVLLALLAPCTEMRRRRIEVC (SEQ ID NO: 24) (referred to as cSIB in Table 8). The examples of peptides designed to be specific inhibitors of importin alpha include AAVALLPAVLLALLAPVELRKAKKDDQMLKRRNVSSF (SEQ ID NO: 25) (referred to as SAR1 in Table 8), AAVALLPAVLLALLAPVELRKNKRDEHLLKRRNVPHE (SEQ ID NO: 26) (referred to as SAR3 in Table 8), AAVALLPAVLLALLAPVELRKNKRDEHLLKKRNVPQE (SEQ ID NO: 27) (referred to as SAR4 in Table 8), AAVALLPAVLLALLAPLQLRKQKREEQLFKRRNVATA (SEQ ID NO: 28) (referred to as SAR5 in Table 8), AAVALLPAVLLALLAPIQLRKQKREQQLFKRRNVELI (SEQ ID NO: 29) (referred to as SAR7 in Table 8), AAVALLPAVLLALLAPCVELRKAKKDDQC (SEQ ID NO: 30) (referred to as cSAR1-C in Table 8), AAVALLPAVLLALLAPCVELRKNKRDEHC (SEQ ID NO: 31) (referred to as cSAR3-C in Table 8), AAVALLPAVLLALLAPCLQLRKQKREEQC (SEQ ID NO: 32) (referred to as cSAR5-C in Table 8), AAVALLPAVLLALLAPCIQLRKQKREQQC (SEQ ID NO: 33) (referred to as cSAR7-C in Table 8), AAVALLPAVLLALLAPCQMLKRRNVSSFC (SEQ ID NO: 34) (referred to as cSAR1-N in Table 8), AAVALLPAVLLALLAPCHLLKRRNVPHEC (SEQ ID NO: 35) (referred to as cSAR3-N in Table 8), AAVALLPAVLLALLAPCHLLKKRNVPQEC (SEQ ID NO: 36) (referred to as cSAR4-N in Table 8), AAVALLPAVLLALLAPCQLFKRRNVATAC (SEQ ID NO: 37) (referred to as cSAR5-N in Table 8), and AAVALLPAVLLALLAPCQLFKRRNVELIC (SEQ ID NO: 38) (referred to as cSAR7-N in Table 8). It is to be understood that any derivatives and/or analogues of these sequences are encompassed by the invention.

In one embodiment, an NTCI as described herein has the sequence AAVALLPAVXLAXXAPVELRKNKRDEHLLKRRNVPHE (SEQ ID NO: 39). Additional NTCIs include SEQ ID NOs: 1-9, 13, and 16-41. It is to be understood that any derivatives and/or analogues of these sequences are encompassed by the invention.

An NTCI as described herein may be an inhibitor of an importin alpha 3 interaction with importin beta.

The SI-3 sequence (Table 8) is designed to block an interaction between importin alpha and importin beta. Hence, this peptide is a cell-penetrating inhibitor of an importin alpha and importin beta interaction. It is to be understood that any derivatives and/or analogues of this sequence is encompassed by the invention.

TABLE 8
Peptide sequences

| | | | SEQ | |
|---|---|---|---|---|
| | | | ID | |
| | SSHR^{§} | Cargo | NO: | Comments |
| SM12 | AAVALLPAVLLALLAP | VQRDEQKLMP | 40 | Importin beta- |
| | | | | selective |
| | | | | inhibitor |
| | | | | (binding studies) |
| SIBB | AAVALLPAVLLALLAP | RRRRIEVNVELRKAKK | 18 | Inhibitor of Imp |
| | | | | alpha 1-importin |
| | | | | beta interaction |
| SI-1 | AAVALLPAVLLALLAP | RRRRIEVNVELRKAKKDD | 19 | Inhibitor of Imp |
| | | | | alpha 1-importin |
| | | | | beta interaction |
| SI-3 | AAVALLPAVLLALLAP | RRQRNEVVVELRKNKRDE | 20 | Inhibitor of Imp |
| | | | | alpha 3-importin |
| | | | | beta interaction |
| SI-4 | AAVALLPAVLLALLAP | RRHRNEVTVELRKNKRDE | 21 | Inhibitor of Imp |
| | | | | alpha 4-importin |
| | | | | beta interaction |
| SI-5 | AAVALLPAVLLALLAP | RRRREEEGLQLRKQKREE | 22 | Inhibitor of Imp |
| | | | | alpha 5-importin |
| | | | | beta interaction |
| SI-7 | AAVALLPAVLLALLAP | RRRREEEGIQLRKQKREQ | 23 | Inhibitor of Imp |
| | | | | alpha 7-importin |
| | | | | beta interaction |
| SAR1 | AAVALLPAVLLALLAP | VELRKAKKDDQMLKRRNV | 25 | Imp alpha 1- |
| | | SSF | | specific |
| SAR3 | AAVALLPAVLLALLAP | VELRKNKRDEHLLKRRNVP | 26 | Imp alpha 3- |
| | | HE | | specific |
| SAR4 | AAVALLPAVLLALLAP | VELRKNKRDEHLLKKRNVP | 27 | Imp alpha 4- |
| | | QE | | specific |
| SAR5 | AAVALLPAVLLALLAP | LQLRKQKREEQLFKRRNVA | 28 | Imp alpha 5- |
| | | TA | | specific |
| SAR7 | AAVALLPAVLLALLAP | IQLRKQKREQQLFKRRNVE | 29 | Imp alpha 7- |
| | | LI | | specific |
| cMN50.1 | AAVALLPAVXLAXXAP | CVQRKRQKLMPC | 17 | Imp alpha 5- |
| | | | | selective |
| cSN50.1β | AAVALLPAVLLALLAP | CVQRDEQKLMPC | 16 | Imp beta- |
| | | | | selective (cell |
| | | | | culture and |
| | | | | preclinical |
| | | | | studies) |
| cSIB | AAVALLPAVLLALLAP | CTEMRRRRIEVC | 24 | Inhibitor of Imp |
| | | | | alpha 1-importin |
| | | | | beta interaction |
| cSAR1-C | AAVALLPAVLLALLAP | CVELRKAKKDDQC | 30 | Imp alpha 1- |
| | | | | specific |
| | | | | Proximal to C- |
| | | | | terminal |
| cSAR3-C | AAVALLPAVLLALLAP | CVELRKNKRDEHC | 31 | Imp alpha 3- |
| | | | | specific |
| | | | | Proximal to C- |
| | | | | terminal |
| cSAR5-C | AAVALLPAVLLALLAP | CLQLRKQKREEQC | 32 | Imp alpha 5- |
| | | | | specific |
| | | | | Proximal to C- |
| | | | | terminal |
| cSAR7-C | AAVALLPAVLLALLAP | CIQLRKQKREQQC | 33 | Imp alpha 7- |
| | | | | specific |
| | | | | Proximal to C- |
| | | | | terminal |
| cSAR1-N | AAVALLPAVLLALLAP | CQMLKRRNVSSFC | 34 | Imp alpha 1- |
| | | | | specific |
| | | | | Proximal to N- |
| | | | | terminal |
| cSAR3-N | AAVALLPAVLLALLAP | CHLLKRRNVPHEC | 35 | Imp alpha 3- |
| | | | | specific |
| | | | | Proximal to N- |
| | | | | terminal |
| cSAR4-N | AAVALLPAVLLALLAP | CHLLKKRNVPQEC | 36 | Imp alpha 4- |
| | | | | specific |
| | | | | Proximal to N- |
| | | | | terminal |
| cSAR5-N | AAVALLPAVLLALLAP | CQLFKRRNVATAC | 37 | Imp alpha 5- |
| | | | | specific |
| | | | | Proximal to N- |
| | | | | terminal |
| cSAR7-N | AAVALLPAVLLALLAP | CQLFKRRNVELIC | 38 | Imp alpha 7- |
| | | | | specific |
| | | | | Proximal to N- |
| | | | | terminal |

### ^{§} Signal Sequence Hydrophobic Region (SSHR)

“Cargo” comprises sequences of functionally active hydrophilic motifs (fragments) listed as linear or cyclized peptides through addition of cysteine at the amino- and carboxy-termini of respective linear peptides. Both linear and cyclized sequences are coupled to hydrophobic membrane translocation motif denoted SSHR.

In one aspect, disclosed herein are methods of reversing the expression profile (i.e., decreasing, reducing, or inhibiting expression when the expression profile increases or increasing expression when the expression profile decreases) of one or more genes encoding mediators of inflammation in a subject whose expression modulates during microbial inflammation as a result of microbial infection (such as, for example, a viral, bacterial, fungal, or parasitic infection) comprising administering to the subject a therapeutically effective amount of a nuclear transport checkpoint inhibitor (NTCI) (such as, for example, SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41).

In one aspect, disclosed herein are methods of reversing the expression profile of one or more genes encoding mediators of inflammation, wherein the one or more genes are selected from the group consisting of I110, Acod1, Saa3, Gpr31b, Fgf23, Igfbp1, Mrgpra2b, Ccl3, Ccl4, Gpr84, Adamts4, Madcam1, Olfm4, Il1f9, Gpr31a, Gpr31c, Ptx3, Slc7a11, Gm6553, Tmprss11g, Tac1, Il1f6, Ccl2, Psg16, Fosl1, Sprr2a2, Serpina3m, Il1rn, Ccl20, Tnf, Gm43181, Clec4e, Gm45774, Ugtla10, Kcnt1, Mt1, Mefv, Clec4d, Ms4a4a, Btbd16, Spic, Orm1, Serpina3f, Il1b, Serpina10, 9830107B12Rik, Gm49839, Msr1, Ankrd2, Saa4, S100a8, Hpx, Apoa4, Steap4, Cxcl10, S100a9, Wfdc17, Gnat1, Fgg, Fgb, Lilr4b, 2010003K11Rik, Serpina3n, AA467197, Gm16685, Timp1, Fga, Ifitm6, Lcn2, F10, Leap2, Retnlg, Ccl7, Ikbke, Vcan, F13a1, Serpina3g, 1200007C13Rik, Ngp, Siglece, Lrrc25, Gm10309, Trem1, Slc39a14, Fgl1, 1127, Tcp10b, Serpina3i, Cxc19, A730049H05Rik, Gm8883, Fpr2, Saa2, Saa1, Prok2, B230303A05Rik, Gm43079, Gm49383, Cldn14, Gm15056, U90926, Stfa2, Prokr2, Lbx1, Cstdc5, Tarm1, Mrgpra2a, Cstdc4, Gm48786, Gm19510, Gm32089, Gm5966, Stfa3, Tmem132e, Orm2, Gm28551, A630023A22Rik, Gm7457, Serpina7, Bmerb1, Klk9, 1700011B04Rik, Asprv1, Dmbt1, Tmprss11d, Slc5a7, Ifitm7, Gm38340, Pglyrp3, Gm26748, 4930431P03Rik, 4732490B19Rik, Sectm1b, Gm4948, Gm20406, Ras110b, Gm36079, Sult2a3, Apcs, Mymk, Nkx2-9, Gm26535, Rnase2a, Slc22a20, Stfa211, Gm4949, Gm11738, Gm29686, Serpine3, Gm807, Gm26444, Trdv5, Sultle1, Serpina3e-ps, 4930578G10Rik, Gm37645, Ceacam19, Ackr1, Ms4a3, B130046B21Rik, Ctcflos, Doc2b, Gm49573, Gm38275, Tex44, Gpr22, Ceacam10, E030018B13Rik, Gm37131, Htr3a, Slc13a5, Nxf3, Gm49427, Pde6h, Ubd, Fcrlb, Sult2a5, Tat, Gm6610, Gm44292, Gm44165, Prss22, Gm11714, Gm49368, Gm48065, Aoah, Tctex1d1, Phox2a, Slc36a3, Sult2a1, Gm37347, Gm50083, Epx, Gm26965, Reg3g, Cela3b, Gm12503, Gpbar1, Dnah6, Fut2, Muc4, Gm43189, Angptl7, Pcdh8, Sftpa1, Mlana, Gm16350, Syt2, Gm17055, Ppy, Ccdc33, Gm33280, Gm45643, Lrit3, Sbk2, Slc6a19os, Slamf1, Gm5541, Fgf21, Gm49392, Slc6a16, Olr1, Gm35028, Slfn4, Gm20548, Gm40709, Speer4f2, Gm13363, Egr2, Gm44260, Marc1, Crygn, Lpo, Gabrp, Bpifc, Fgf18, Lncenc1, Prss27, Muc19, Gm39929, Gm2670, 5830487J09Rik, Olfr1442, Krt20, Gm19684, Gm33424, 4732419C18Rik, Gm37621, Creb313, Gm14319, Dnah10, Areg, Armc12, Eif4ebp3, Gdf6, Gm49037, Gm42793, Der13, Nptx2, Atp12a, Kcna7, Prg4, Ctla4, Gm44164, Mmp7, Gm44949, Slc17a8, Lamb3, Plekhs1, Trim17, Dusp8, 4930422C21Rik, Gm10727, Col27a1, Tacc2, Fat2, Lif, Klk1b1, Tsc22d1, Wnt8b, Grem1, Hesx1, Hhipl1, Ugtla5, Klk1b4, Gm8947, A530013C23Rik, Mylk4, Cyp2a5, Muc1, Gm44588, Gm45053, Ano5, Gsg11, Spon2, Oxtr, Rnf112, Ppp2r2c, Crabp1, St6galnac3, Agtr2, Gjc2, Gm33195, Gabra4, Aplnr, E030013I19Rik, Pcdh12, Chp2, Gng8, Pla2g2d, Gm20683, Mansc4, Gm10824, Ces2b, Kank4, Zmat4, Cdr1os, Wnt2, Gm17040, Wfdc16, Rab42, Tbxa2r, Plppr5, Misp3, Mmp28, Lrrn4, Sost, Tmem200a, Shisa2, Stmn2, Chrm1, Tmeff2, 2810433D01Rik, Pabpc41, Dact3, Mett124, Gpr34, D430019H16Rik, Hoxc5, Tekt5, Megf10, Wscd1, Gm32219, Tril, Agtr1b, 2610203C22Rik, Abcg3, Star, Grik2, Grin2c, Vstm5, Smad9, Pianp, Rnf144a, Cthrc1, Nxpe4, Mrc2, Cx3cr1, 115ra, Kcnc4, Fam163a, Cxcr3, A330074K22Rik, Gm11627, Mmp11, Fam181b, Cd209b, Tnfrsf19, Sept4, Nlgn3, Baalc, Ngb, Nxph3, Klb, Igkv4-72, Ptprv, Nrep, Itga8, Myct1, Palm, Syt3, Tspan18, Piezo2, Nat8f3, Cngb1, Cd209g, Faim2, Gfra2, Srpk3, Gm3716, Klhl13, Mogat2, Tmem233, Duxb13, Rnase1, Defb42, AL935121.1, Zp1, Snurf, Gm25541, 4933403008Rik, 1810046K07Rik, Gm5639, Myh2, Gm765, Gm50242, Gm6018, Ackr4, Tnfrsf17, Gm37858, Igkv14-130, Ptprh, Gpr165, Gm47652, Gm16534, 0610012D04Rik, Gm36569, Gzmk, Gm9918, Gm33677, St6gal2, Krtap17-1, Olfr1342, Lhcgr, Ccdc42, Zfp985, Igkv4-70, Gm31517, Gm9767, Olfr71, Slc26a3, Trpv3, Gm49727, Gm43951, E330018M18Rik, Calcb, Rspo2, Glp1r, Fam124b, Amtn, 4930570G19Rik, Gm11574, Pla2g2e, Sfrp2, Pigz, Gm13688, Abca14, B530045E10Rik, Fras1, Gm12159, Gm48488, 9030404E10Rik, Hmcn1, Igkv4-68, Wnt7a, Tmem74, Ighv1-66, Cr2, Slc5a11, Lrrc17, Slc22a14, Dipk2b, Fgf18, Ndp, Gm17334, Btnl2, Gm10432, Colq, Gm48662, 4833412C05Rik, Gm12576, Igkv6-17, Gm13816, Tmem266, Ighv5-6, Cntn4, Rbp7, Insyn2a, Slc5a12, Gm9947, Igkv12-89, Gm26795, Ddx4, Dclk3, Hpcal4, Ndnf, Igkv17-121, Slurp1, Ano4, Ighv1-7, Rimklb, Igkv4-80, Cyp11b1, Cyp11a1, Akr1cl, Hsd3b1, Scg2, Th, Cyp21a1, Chga, Cyp21a2-ps, Pnmt, Chgb, Cyp11b2, Caln1, Kcnk9, Dbh, Gm28382, Pla2g2f, Nr5a1, Pcskln, Mgarp, 4930486L24Rik, Cryba4, Atp2b3, Resp18, E330017L17Rik, Pcsk1, Snap25, Rims4, Slc24a2, Phox2b, Slc18a2, Eef1a2, Chrna3, Hand2os1, Cntnap5a, Rasgrf1, Hand2, Pnmal1, Necab2, Tbx20, Stmn3, Gm17322, St8sia3, Rab33a, Synpr, Rit2, Camkv, Gdap1, Soga3, Mdga2, Mab2112, Nrsn1, Cadps, Gnb3, 1520401A03Rik, Nrg3, Spock1, Cdk5r2, Ina, Actl6b, Npy2r, Gal, Nrsn2, Gabrg2, Gpr158, Slc7a14, Sphkap, Tmem255a, 8430419K02Rik, Scg3, Gria1, Txndc2, Gm27477, Gria2, Gjd2, Lrrtm2, Hsd3b6, Diras1, Omg, Gm49391, Fam155a, Gm2007, Kcnq2, Gm42372, Is11, Lingo3, Prl8a2, Adh7, Lgi1, Gm47815, Gm14592, Cdx4, Marcks11-ps4, Sstr3, Pcdh10, Gm27000, Tub, Mc2r, 1120ra, Grin2a, IL-1B, IL-10, Tnf-α, Ifn-γ, Nos1, Sele, Sell, Selp, Acat3, Tnfrsf1b, Csf1, Cx3cl1, and Ptgs2, Ccl12, Ccr2,Ccr5, Cxcl11, and/or Isg15.

Also disclosed herein are methods of reversing the expression profile of one or more genes encoding mediators of inflammation, wherein the expression of the one or more genes increases during microbial inflammation and wherein the one or more genes are selected from the group consisting of I110, Acod1, Saa3, Gpr31b, Fgf23, Igfbp1, Mrgpra2b, Ccl3, Ccl4, Gpr84, Adamts4, Madcam1, Olfm4, Il1f9, Gpr31a, Gpr31c, Ptx3, Slc7a11, Gm6553, Tmprss11g, Tac1, Il1f6, Ccl2, Psg16, Fosl1, Sprr2a2, Serpina3m, Il1rn, Cc120, Tnf, Gm43181, Clec4e, Gm45774, Ugtla10, Kcnt1, Mt1, Mefv, Clec4d, Ms4a4a, Btbd16, Spic, Orm1, Serpina3f, Il1b, Serpina10, 9830107B12Rik, Gm49839, Msr1, Ankrd2, Saa4, S100a8, Hpx, Apoa4, Steap4, Cxcl10, S100a9, Wfdc17, Gnat1, Fgg, Fgb, Lilr4b, 2010003K11Rik, Serpina3n, AA467197, Gm16685, Timp1, Fga, Ifitm6, Lcn2, F10, Leap2, Retnlg, Ccl7, Ikbke, Vcan, F13a1, Serpina3g, 1200007C13Rik, Ngp, Siglece, Lrrc25, Gm10309, Trem1, Slc39a14, Fgl1, 1127, Tcp10b, Serpina3i, Cxc19, A730049H05Rik, Gm8883, Fpr2, Saa2, Saa1, Prok2, B230303A05Rik, Gm43079, Gm49383, Cldn14, Gm15056, U90926, Stfa2, Prokr2, Lbx1, Cstdc5, Tarm1, Mrgpra2a, Cstdc4, Gm48786, Gm19510, Gm32089, Gm5966, Stfa3, Tmem132e, Orm2, Gm28551, A630023A22Rik, Gm7457, Serpina7, Bmerb1, Klk9, 1700011B04Rik, Asprv1, Dmbt1, Tmprss11d, Slc5a7, Ifitm7, Gm38340, Pglyrp3, Gm26748, 4930431P03Rik, 4732490B19Rik, Sectm1b, Gm4948, Gm20406, Rasl10b, Gm36079, Sult2a3, Apcs, Mymk, Nkx2-9, Gm26535, Rnase2a, Slc22a20, Stfa211, Gm4949, Gm11738, Gm29686, Serpine3, Gm807, Gm26444, Trdv5, Sultle1, Serpina3e-ps, 4930578G10Rik, Gm37645, Ceacam19, Ackr1, Ms4a3, B130046B21Rik, Ctcflos, Doc2b, Gm49573, Gm38275, Tex44, Gpr22, Ceacam10, E030018B13Rik, Gm37131, Htr3a, Slc13a5, Nxf3, Gm49427, Pde6h, Ubd, Fcrlb, Sult2a5, Tat, Gm6610, Gm44292, Gm44165, Prss22, Gm11714, Gm49368, Gm48065, Aoah, Tctex1d1, Phox2a, Slc36a3, Sult2a1, Gm37347, Gm50083, Epx, Gm26965, Reg3g, Cela3b, Gm12503, Gpbar1, Dnah6, Fut2, Muc4, Gm43189, Angpt17, Pcdh8, Sftpa1, Mlana, Gm16350, Syt2, Gm17055, Ppy, Ccdc33, Gm33280, Gm45643, Lrit3, Sbk2, Slc6a19os, Slamf1, Gm5541, Fgf21, Gm49392, Slc6a16, Olr1, Gm35028, Slfn4, Gm20548, Gm40709, Speer4f2, Gm13363, Egr2, Gm44260, Marc1, Crygn, Lpo, Gabrp, Bpifc, Fgf18, Lncenc1, Prss27, Muc19, Gm39929, Gm2670, 5830487J09Rik, Olfr1442, Krt20, Gm19684, Gm33424, 4732419C18Rik, Gm37621, Creb313, Gm14319, Dnah10, Areg, Armc12, Eif4ebp3, Gdf6, Gm49037, Gm42793, Der13, Nptx2, Atp12a, Kcna7, Prg4, Ctla4, Gm44164, Mmp7, Gm44949, Slc17a8, Lamb3, Plekhs1, Trim17, Dusp8, 4930422C21Rik, Gm10727, Col27a1, Tacc2, Fat2, Lif, Klk1b1, Tsc22d1, Wnt8b, Grem1, Hesx1, Hhipl1, Ugtla5, Klk1b4, Gm8947, A530013C23Rik, Mylk4, Cyp2a5, Muc1, Gm44588, Gm45053, IL-1B, IL-10, Tnf-α, Ifn-γ, Nos1, Sele, Sell, Selp, Acat3, Tnfrsf1b, Csf1, Cx3cl1, and Ptgs2, Ccl12, Ccr2, Ccr5, Cxcl11, and/or Isg15.

In one aspect, disclosed herein are methods of reversing the expression profile of one or more genes encoding mediators of inflammation, wherein the expression of the one or more genes decreases during microbial inflammation and wherein the one or more genes are selected from the group consisting of Ano5, Gsg11, Spon2, Oxtr, Rnf112, Ppp2r2c, Crabp1, St6galnac3, Agtr2, Gjc2, Gm33195, Gabra4, Aplnr, E030013I19Rik, Pcdh12, Chp2, Gng8, Pla2g2d, Gm20683, Mansc4, Gm10824, Ces2b, Kank4, Zmat4, Cdr1os, Wnt2, Gm17040, Wfdc16, Rab42, Tbxa2r, Plppr5, Misp3, Mmp28, Lrrn4, Sost, Tmem200a, Shisa2, Stmn2, Chrm1, Tmeff2, 2810433D01Rik, Pabpc41, Dact3, Mett124, Gpr34, D430019H16Rik, Hoxc5, Tekt5, Megf10, Wscd1, Gm32219, Tril, Agtr1b, 2610203C22Rik, Abcg3, Star, Grik2, Grin2c, Vstm5, Smad9, Pianp, Rnf144a, Cthrc1, Nxpe4, Mrc2, Cx3cr1, Il5ra, Kcnc4, Fam163a, Cxcr3, A330074K22Rik, Gm11627, Mmp11, Fam181b, Cd209b, Tnfrsf19, Sept4, Nlgn3, Baalc, Ngb, Nxph3, Klb, Igkv4-72, Ptprv, Nrep, Itga8, Myct1, Palm, Syt3, Tspan18, Piezo2, Nat8f3, Cngb1, Cd209g, Faim2, Gfra2, Srpk3, Gm3716, Klhl13, Mogat2, Tmem233, Duxb13, Rnase1, Defb42, AL935121.1, Zp1, Snurf, Gm25541, 4933403008Rik, 1810046K07Rik, Gm5639, Myh2, Gm765, Gm50242, Gm6018, Ackr4, Tnfrsf17, Gm37858, Igkv14-130, Ptprh, Gpr165, Gm47652, Gm16534, 0610012D04Rik, Gm36569, Gzmk, Gm9918, Gm33677, St6gal2, Krtap17-1, Olfr1342, Lhcgr, Ccdc42, Zfp985, Igkv4-70, Gm31517, Gm9767, Olfr71, Slc26a3, Trpv3, Gm49727, Gm43951, E330018M18Rik, Calcb, Rspo2, Glp1r, Fam124b, Amtn, 4930570G19Rik, Gm11574, Pla2g2e, Sfrp2, Pigz, Gm13688, Abca14, B530045E10Rik, Fras1, Gm12159, Gm48488, 9030404E10Rik, Hmcn1, Igkv4-68, Wnt7a, Tmem74, Ighv1-66, Cr2, Slc5a11, Lrrc17, Slc22a14, Dipk2b, Fgf18, Ndp, Gm17334, Btn12, Gm10432, Colq, Gm48662, 4833412C05Rik, Gm12576, Igkv6-17, Gm13816, Tmem266, Ighv5-6, Cntn4, Rbp7, Insyn2a, Slc5a12, Gm9947, Igkv12-89, Gm26795, Ddx4, Dclk3, Hpcal4, Ndnf, Igkv17-121, Slurp1, Ano4, Ighv1-7, Rimklb, Igkv4-80, Cyp11b1, Cyp11a1, Akr1c1, Hsd3b1, Scg2, Th, Cyp21a1, Chga, Cyp21a2-ps, Pnmt, Chgb, Cyp11b2, Caln1, Kcnk9, Dbh, Gm28382, Pla2g2f, Nr5a1, Pcskln, Mgarp, 4930486L24Rik, Cryba4, Atp2b3, Resp18, E330017L17Rik, Pcsk1, Snap25, Rims4, Slc24a2, Phox2b, Slc18a2, Eef1a2, Chrna3, Hand2os1, Cntnap5a, Rasgrf1, Hand2, Pnmal1, Necab2, Tbx20, Stmn3, Gm17322, St8sia3, Rab33a, Synpr, Rit2, Camkv, Gdap1, Soga3, Mdga2, Mab2112, Nrsn1, Cadps, Gnb3, 1520401A03Rik, Nrg3, Spock1, Cdk5r2, Ina, Actl6b, Npy2r, Gal, Nrsn2, Gabrg2, Gpr158, Slc7a14, Sphkap, Tmem255a, 8430419K02Rik, Scg3, Gria1, Txndc2, Gm27477, Gria2, Gjd2, Lrrtm2, Hsd3b6, Diras1, Omg, Gm49391, Fam155a, Gm2007, Kcnq2, Gm42372, Is11, Lingo3, Prl8a2, Adh7, Lgi1, Gm47815, Gm14592, Cdx4, Marcks11-ps4, Sstr3, Pcdh10, Gm27000, Tub, Mc2r, Il20ra, and/or Grin2a.

It is understood and herein contemplated that the gene expression profile of genes modulated by microbial inflammation can vary between organs and the effect of one or more NTCI on said genes can also vary between organs. Accordingly, in one aspect, disclosed herein are methods of reversing the expression profile of one or more genes encoding mediators of inflammation, wherein the microbial inflammation is localized in the lungs; wherein the expression of the one or more genes increases during microbial inflammation; and wherein the one or more genes are selected from the group consisting of I110, Acod1, Saa3, Gpr31b, Fgf23, Igfbp1, Mrgpra2b, Ccl3, Ccl4, Gpr84, Adamts4, Madcam1Olfm4, Il1f9, Gpr31a, Gpr31c, Ptx3, Slc7a11, Gm6553, Tmprss11g, Tac1, Il1f6, Ccl2, Psg16, Fosl1, Sprr2a2, Serpina3m, Il1rn, Cc120, Tnf, Gm43181, Clec4e, Gm45774, Ugtla10, Kcnt1, Mt1, Mefv, Clec4d, Ms4a4a, Btbd16, Spic, Orm1, Serpina3f, Il1b, Serpina10, 9830107B12Rik, Gm49839, Msr1, Ankrd2, Saa4, S100a8, Hpx, Apoa4, Steap4, Cxcl10, S100a9, Wfdc17, Gnat1, Fgg, Fgb, Lilr4b, 2010003K11Rik, Serpina3n, AA467197, Gm16685, Timp1, Fga, Ifitm6, Lcn2, F10, Leap2, Retnlg, Ccl7, Ikbke, Vcan, F13a1, Serpina3g, 1200007C13Rik, Ngp, Siglece, Lrrc25, Gm10309, Trem1, Slc39a14, Fgl1, 1127, Tcp10b, Serpina3i, Cxcl9, A730049H05Rik, Gm8883, Fpr2, Saa2, Saa1, Prok2, B230303A05Rik, Gm43079, Gm49383, Cldn14, Gm15056, U90926, Stfa2, Prokr2, Lbx1, Cstdc5, Tarm1, Mrgpra2a, Cstdc4, Gm48786, Gm19510, Gm32089, Gm5966, Stfa3, Tmem132e, Orm2, Gm28551, A630023A22Rik, Gm7457, Serpina7, Bmerb1, Klk9, 1700011B04Rik, Asprv1, Dmbt1, Tmprss11d, Slc5a7, Ifitm7, Gm38340, Pglyrp3, Gm26748, 4930431P03Rik, 4732490B19Rik, Sectm1b, Gm4948, Gm20406, Ras110b, Gm36079, Sult2a3, Apcs, Mymk, Nkx2-9, Gm26535, Rnase2a, Slc22a20, Stfa211, Gm4949, Gm11738, Gm29686, Serpine3, Gm807, Gm26444, Trdv5, Sultle1, Serpina3e-ps, 4930578G10Rik, Gm37645, Ceacam19, Ackr1, Ms4a3, B130046B21Rik, Ctcflos, Doc2b, Gm49573, Gm38275, Tex44, Gpr22, Ceacam10, E030018B13Rik, Gm37131, Htr3a, Slc13a5, Nxf3, Gm49427, Pde6h, Ubd, Fcrlb, Sult2a5, Tat, Gm6610, Gm44292, Gm44165, Prss22, Gm11714, Gm49368, Gm48065, Aoah, Tctex1d1, Phox2a, Slc36a3, Sult2a1, Gm37347, Gm50083, IL-1B, IL-10, Tnf-α, Ifn-γ, Nos1, Sele, Sell, Selp, Acat3, Tnfrsf1b, Csf1, Cx3cl1, and Ptgs2, Ccl12, Ccr2,Ccr5, Cxcl11, and/or Isg15.

Also disclosed herein are methods of reversing the expression profile of one or more genes encoding mediators of inflammation, wherein the microbial inflammation is localized in the lungs; wherein the expression of the one or more genes decreases during microbial inflammation and wherein the one or more genes are selected from the group consisting of Ano5, Gsg11, Spon2, Oxtr, Rnf112, Ppp2r2c, Crabp1, St6galnac3, Agtr2, Gjc2, Gm33195, Gabra4, Aplnr, E030013I19Rik, Pcdh12, Chp2, Gng8, Pla2g2d, Gm20683, Mansc4, Gm10824, Ces2b, Kank4, Zmat4, Cdr1os, Wnt2, Gm17040, Wfdc16, Rab42, Tbxa2r, Plppr5, Misp3, Mmp28, Lrrn4, Sost, Tmem200a, Shisa2, Stmn2, Chrm1, Tmeff2, 2810433D01Rik, Pabpc41, Dact3, Mett124, Gpr34, D430019H16Rik, Hoxc5, Tekt5, Megf10, Wscd1, Gm32219, Tril, Agtr1b, 2610203C22Rik, Abcg3, Star, Grik2, Grin2c, Vstm5, Smad9, Pianp, Rnf144a, Cthrc1, Nxpe4, Mrc2, Cx3cr1, Il5ra, Kcnc4, Fam163a, Cxcr3, A330074K22Rik, Gm11627, Mmp11, Fam181b, Cd209b, Tnfrsf19, Sept4, Nlgn3, Baalc, Ngb, Nxph3, Klb, Igkv4-72, Ptprv, Nrep, Itga8, Myct1, Palm, Syt3, Tspan18, Piezo2, Nat8f3, Cngb1, Cd209g, Faim2, Gfra2, Srpk3, Gm3716, Klhl13, Mogat2, Tmem233, Duxb13, Rnase1, Defb42, AL935121.1, Zp1, Snurf, Gm25541, 4933403008Rik, 1810046K07Rik, Gm5639, Myh2, Gm765, Gm50242, Gm6018, Ackr4, Tnfrsf17, Gm37858, Igkv14-130, Ptprh, Gpr165, Gm47652, Gm16534, 0610012D04Rik, Gm36569, Gzmk, Gm9918, Gm33677, St6gal2, Krtap17-1, Olfr1342, Lhcgr, Ccdc42, Zfp985, Igkv4-70, Gm31517, Gm9767, Olfr71, Slc26a3, Trpv3, Gm49727, Gm43951, E330018M18Rik, Calcb, Rspo2, Glp1r, Fam124b, Amtn, 4930570G19Rik, Gm11574, Pla2g2e, Sfrp2, Pigz, Gm13688, Abca14, B530045E10Rik, Fras1, Gm12159, Gm48488, 9030404E10Rik, Hmcn1, Igkv4-68, Wnt7a, Tmem74, Ighv1-66, Cr2, Slc5al1, Lrrc17, Slc22a14, Dipk2b, Fgf18, Ndp, Gm17334, Btn12, Gm10432, Colq, Gm48662, 4833412C05Rik, Gm12576, Igkv6-17, Gm13816, Tmem266, Ighv5-6, Cntn4, Rbp7, Insyn2a, Slc5a12, Gm9947, Igkv12-89, Gm26795, Ddx4, Dclk3, Hpcal4, Ndnf, Igkv17-121, Slurp1, Ano4, Ighv1-7, Rimklb, and/or Igkv4-80.

In one aspect, disclosed herein are methods of reversing the expression profile of one or more genes encoding mediators of inflammation, wherein the microbial inflammation is localized in the kidneys; wherein the expression of the one or more genes increases during microbial inflammation and wherein the one or more genes are selected from the group consisting of Acod1, Saa3, Gpr31b, Fgf23, Igfbp1, Mrgpra2b, Ccl3, Ccl4, Gpr84, Adamts4, Madcam1, Olfm4, Il1f9, Gpr31a, Gpr31c, Ptx3, Slc7a11, Gm6553, Tmprss11g, Tac1, Il1f6, Ccl2, Psg16, Sprr2a2, Serpina3m, Il1rn, Ccl20, Tnf, Gm43181, Clec4e, Gm45774, Ugtla10, Kcnt1, Mt1, Mefv, Clec4d, Ms4a4a, Btbd16, Spic, Orm1, Serpina3f, Il1b, Serpina10, 9830107B12Rik, Gm49839, Msr1, Ankrd2, Saa4, S100a8, Hpx, Apoa4, Steap4, Cxcl10, S100a9, Wfdc17, Gnat1, Fgg, Fgb, Lilr4b, 2010003K11Rik, Serpina3n, AA467197, Gm16685, Timp1, Fga, Ifitm6, Lcn2, F10, Leap2, Retnlg, Ccl7, Ikbke, Vcan, F13a1, Serpina3g, 1200007C13Rik, Ngp, Siglece, Lrrc25, Gm10309, Trem1, Slc39a14, Fgl1, 1127, Tcp10b, Serpina3i, Cxc19, A730049H05Rik, Gm8883, Fpr2, Epx, Gm26965, Reg3g, Cela3b, Gm12503, Gpbar1, Dnah6, Fut2, Muc4, Gm43189, Angpt17, Pcdh8, Sftpa1, Mlana, Gm16350, Syt2, Gm17055, Ppy, Ccdc33, Gm33280, Gm45643, Lrit3, Sbk2, Slc6a19os, Slamf1, Gm5541, Fgf21, Gm49392, Slc6a16, Olr1, Gm35028, Slfn4, Gm20548, Gm40709, Speer4f2, Gm13363, Egr2, Gm44260, Marc1, Crygn, Lpo, Gabrp, Bpifc, Fgf18, Lncenc1, Prss27, Muc19, Gm39929, Gm2670, 5830487J09Rik, Olfr1442, Krt20, Gm19684, Gm33424, 4732419C18Rik, Gm37621, Creb313, Gm14319, Dnah10, Areg, Armc12, Eif4ebp3, Gdf6, Gm49037, Gm42793, Der13, Nptx2, Atp12a, Kcna7, Prg4, Ctla4, Gm44164, Mmp7, Gm44949, Slc17a8, Lamb3, Plekhs1, Trim17, Dusp8, 4930422C21Rik, Gm10727, Col27a1, Tacc2, Fat2, Lif, Klk1b1, Tsc22d1, Wnt8b, Grem1, Hesx1, Hhipl1, Ugtla5, Klk1b4, Gm8947, A530013C23Rik, Mylk4, Cyp2a5, Muc1, Gm44588, Gm45053, IL-1B, IL-10, Tnf-α, Ifn-γ, Nos1, Sele, Sell, Selp, Acat3, Tnfrsf1b, Csf1, Cx3cl1, and Ptgs2, Ccl12, Ccr2,Ccr5, Cxcl11, and/or Isg15.

Also disclosed herein are methods of reversing the expression profile of one or more genes encoding mediators of inflammation, wherein the microbial inflammation is localized in the kidneys; wherein the expression of the one or more genes decreases during microbial inflammation and wherein the one or more genes are selected from the group consisting of Ano5, Gsg11, Spon2, Oxtr, Rnf112, Ppp2r2c, Crabp1, St6galnac3, Agtr2, Gjc2, Gm33195, Gabra4, Aplnr, E030013I19Rik, Pcdh12, Chp2, Gng8, Pla2g2d, Gm20683, Mansc4, Gm10824, Ces2b, Kank4, Zmat4, Cdr1os, Wnt2, Gm17040, Wfdc16, Rab42, Tbxa2r, Plppr5, Misp3, Mmp28, Lrrn4, Sost, Tmem200a, Shisa2, Stmn2, Chrm1, Tmeff2, 2810433D01Rik, Pabpc41, Dact3, Mett124, Gpr34, D430019H16Rik, Hoxc5, Tekt5, Megf10, Wscd1, Gm32219, Tril, Agtr1b, 2610203C22Rik, Abcg3, Star, Grik2, Grin2c, Vstm5, Smad9, Pianp, Rnf144a, Cthrc1, Nxpe4, Mrc2, Cx3cr1, Il5ra, Kcnc4, Fam163a, Cxcr3, A330074K22Rik, Gm11627, Mmp11, Fam181b, Cd209b, Tnfrsf19, Sept4, Nlgn3, Baalc, Ngb, Nxph3, Klb, Igkv4-72, Ptprv, Nrep, Itga8, Myct1, Palm, Syt3, Tspan18, Piezo2, Nat8f3, Cngb1, Cd209g, Faim2, Gfra2, Srpk3, Gm3716, Klh113, Mogat2, Cyp11b1, Cyp11a1, Akr1cl, Hsd3b1, Scg2, Th, Cyp21a1, Chga, Cyp21a2-ps, Pnmt, Chgb, Cyp11b2, Caln1, Kcnk9, Dbh, Gm28382, Pla2g2f, Nr5a1, Pcskln, Mgarp, 4930486L24Rik, Cryba4, Atp2b3, Resp18, E330017L17Rik, Pcsk1, Snap25, Rims4, Slc24a2, Phox2b, Slc18a2, Eef1a2, Chrna3, Hand2os1, Cntnap5a, Rasgrf1, Hand2, Pnmal1, Necab2, Tbx20, Stmn3, Gm17322, St8sia3, Rab33a, Synpr, Rit2, Camkv, Gdap1, Soga3, Mdga2, Mab2112, Nrsn1, Cadps, Gnb3, 1520401A03Rik, Nrg3, Spock1, Cdk5r2, Ina, Act16b, Npy2r, Gal, Nrsn2, Gabrg2, Gpr158, Slc7a14, Sphkap, Tmem255a, 8430419K02Rik, Scg3, Gria1, Txndc2, Gm27477, Gria2, Gjd2, Lrrtm2, Hsd3b6, Diras1, Omg, Gm49391, Fam155a, Gm2007, Kcnq2, Gm42372, Is11, Lingo3, Prl8a2, Adh7, Lgi1, Gm47815, Gm14592, Cdx4, Marcks11-ps4, Sstr3, Pcdh10, Gm27000, Tub, Mc2r, Il20ra, and/or Grin2a.

Also disclosed herein are methods of reversing the expression profile of one or more genes encoding mediators of inflammation, wherein the microbial inflammation is acute inflammation, subacute inflammation, chronic inflammation, organ-specific inflammation, systemic inflammation, sepsis, or polymicrobial sepsis.

In one aspect, it is understood that control of aberrant gene expression in sepsis can have the therapeutic effect of treating conditions associated with gene over expression or suppression during microbial inflammation. For example, Intensive Care Unit-Acquired Weakness (ICUAW), that delays patients' recovery being mediated by Saa1 gene-encoded Serum Amyloid A1 contributing to the myotube injury can be treated by administering an NTCI. In addition, another sepsis-suppressed gene linked to muscle dystrophy, anoctamin 5 (Ano5), is beneficially regulated by the NTCI, Hence, the treatment with the NTCI lowered the expression of Saa1 Saa3 gene in the lungs and the kidneys and raised the expression of Anoctamin 5 gene. Similarly, NTCI can be used in the treatment of Disseminated intravascular coagulation (DIC), one of the major hematologic complications of sepsis. As shown herein the NTCI reduced the expression of the genes encoding fibrinogen chains and Factor X in the lungs and kidneys potentially reducing the risk of DIC in sepsis.

It is understood and herein contemplated that the microbial inflammation which mediates inflammation (including, but not limited to sepsis and polymicrobial sepsis) being treated can be in any tissue, organ, or system in the subject where a microbial infection can take place, including, but not limited to the blood, brain, sinuses, upper respiratory tract, or lungs heart, bone marrow, spleen, liver, kidneys, genito-urinary tract, bladder, aural cavities, stomach, intestines, skin, eyes, teeth, or gingiva. Accordingly, in one aspect, disclosed herein are methods of reversing the expression profile of one or more genes encoding mediators of inflammation, wherein the microbial inflammation is localized in the blood, brain, sinuses, upper respiratory tract, or lungs, heart, bone marrow, spleen, liver, kidneys, genito-urinary tract, bladder, aural cavities, stomach, intestines, skin, eyes, teeth, and/or gingiva.

“Microbial inflammation” refers to a physical condition associated with its cardinal signs such as redness, swelling, increase in temperature, pain, and impairment of organ function such as impaired respiration as a result of the microvascular endothelial injury in the lungs (and other organs) due to a microbial infection such as a virus, bacteria, fungi, or parasite. The innate and adaptive immune response to infecting pathogen (disease-causing microorganism) can include the burst in production of cytokines, chemokines, and proteolytic enzymes by granulocytes, monocytes, macrophages, dendritic cells, mast cells, innate lymphoid cells, T cells, B cells, NK cells, and NK T cells. Microbial inflammation can be localized to a specific organ- or can be systemic. Microbial inflammation can proceed in stages from acute to subacute and chronic with attendant tissue destruction and subsequent fibrosis. Left unchecked, the acute microbial inflammation can lead to sepsis and septic shock, the end stage of microbial inflammation.

“Microbial inflammation” refers to a mechanism of disease caused by infection (“microbial insult”). It evolves from innate immune response to an infection due to a microbe such as, for example, a virus, bacterium, fungus, or parasite. Thus, the microbial injury caused by microbial virulence factors is aggravated by the host-produced inflammatory mediators that impede the clearance of invading microbes and add insult to organ's injury. It is understood and herein contemplated that the microbial inflammation and its end stage, sepsis can result from any microbial insult elicited by known (or unknown) virulence factors and microbial antigens.

“Pathogen” is an agent that causes infection or disease, especially a virus, bacterium, fungus, protozoa, or parasite.

In one aspect, it is understood that the pathogen can be a virus. Accordingly, also disclosed herein are methods of reversing the expression profile of one or more genes encoding mediators of inflammation, wherein the microbial inflammation is caused by a viral infection caused by Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian

Immunodeficiency virus, Human T-cell Leukemia virus type-1 and 2, Hantavirus, Rubella virus, Simian Immunodeficiency virus, Human Immunodeficiency virus type-1, or Human Immunodeficiency virus type-2.

Similarly, it is understood that the pathogen can be a bacterium. Thus, in one aspect, disclosed herein are methods of reversing the expression profile of one or more genes encoding mediators of inflammation, wherein the microbial inflammation is caused by a bacterial infection caused by Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, or other Yersinia species. In some aspects, the bacteria causing the bacterial infection is not Bacillus anthracis.

Additionally, the pathogen can be a fungus. Therefore, also disclosed herein are methods of reversing the expression profile of one or more genes encoding mediators of inflammation, wherein the microbial inflammation is caused by a fungal infection caused by a fungi including, but not limited to Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

In one aspect, the pathogen can be a parasite. Thus, also disclosed herein are methods of reversing the expression profile of one or more genes encoding mediators of inflammation, wherein the microbial inflammation is caused by a parasitic infection including, but not limited to an infection of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

As shown herein, the disclosed NTCI (such as, for example, SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41) can effectuate the clearance of an infecting microbe even without the presence of an anti-microbial agent. This clearance can be the result of the ability of the NTCI to control the inflammatory response so the host immune system can remove the infecting microbe whereas without the NTCI, uncontrolled inflammation (such as, for example, acute inflammation, subacute inflammation, chronic inflammation, organ-specific inflammation, systemic inflammation, and/or sepsis including, but not limited to polymicrobial sepsis) would proceed, increase, or continue, or the clearance can occur by enhancing innate immunity-mediated suppression virulence factor production or reduction of virulence factors production by the infecting pathogen such a virus, fungus, or protozoa/parasite. Furthermore, it is understood and herein contemplated that as the disclosed NTCI (such as, for example, SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41) can, without the aid of an anti-microbial agent, reduce the numbers of infecting microbes and alter the inflammatory milieu such that the host immune system can appropriately fight the infecting microbe. Accordingly, in one aspect, disclosed herein are methods of reducing, decreasing, removing, clearing, and/or eliminating a microbial infection (such as, for example, a viral, bacterial, fungal, or parasitic infection) and/or the microbial burden (i.e., clearing the microbe) in the blood, brain, upper respiratory tract including sinuses, and/or lungs, heart, bone marrow, spleen, liver, kidneys, genito-urinary tract, bladder, aural cavities, stomach, intestines, skin, eyes, teeth, and gingiva comprising administering to the subject a therapeutically effective amount of a composition comprising one or more NTCI (such as, for example, SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41). In one aspect, the methods of reducing/inhibiting the presence of a pathogenic microbe (i.e., clearing the microbe) comprise administering to a subject a composition comprising an NTCI but said method does not comprise the administration of an anti-microbial agent. In one aspect, the methods of reducing/inhibiting the presence of a pathogenic microbe (i.e., clearing the microbe) can further comprise the administration of an anti-microbial agent. As used herein, clearance refers to a reduction in the number of infecting microbes at a site of infection such as a tissue, organ, or system (such as for example, blood, upper respiratory tract including sinuses, and/or lungs, brain, bone marrow, spleen, liver, kidneys, genito-urinary tract, bladder, aural cavities, stomach, intestines, skin, eyes, teeth and gingiva,). It is understood and herein contemplated that clearance while including the complete or partial elimination of the infecting pathogenic microbe can include less robust reductions in the infecting microbes. Thus, clearance can include such as a 10, 20, 25, 30, 33, 45, 40, 45, 50, 55, 60, 66, 70, 75, 80, 85, 90, 95, 96, 97, 98, or 99% reduction in the numbers of the infecting microbe.

It is understood and herein contemplated that the anti-inflammatory effects observed as a result of the disclosed compositions and used in the disclosed methods treating microbial inflammation can have a therapeutic effect even without the presence of any anti-microbial agent being administered in a composition with and/or in a separate composition from the NTCI. Thus, in one aspect, disclosed herein are methods of reducing, decreasing, removing, clearing, and/or eliminating a microbial infection (such as, for example, a viral, bacterial, fungal, or parasitic infection) and/or the microbial burden of the microbial infection in a subject comprising administering to the subject an NTCI (such as, for example, SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41), wherein the methods does not comprise administering an anti-microbial agent either as part of the NTCI composition or as a separate administration.

Also disclosed herein are methods of reducing, decreasing, removing, clearing, and/or eliminating a microbial infection and/or the microbial burden of the microbial infection (i.e., clearing the microbe), wherein the microbial burden is caused by a viral infection caused by Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

In some aspects, disclosed herein are methods of reducing, decreasing, removing, clearing, and/or eliminating a microbial infection and/or the microbial burden of the microbial infection (i.e., clearing the microbe), wherein the microbial burden is caused by a bacterial infection caused by an infection with a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species. In some aspects, the bacteria causing the bacterial infection is not Bacillus anthracis.

Also disclosed herein are methods of reducing, decreasing, removing, clearing, and/or eliminating a microbial infection and/or the microbial burden of the microbial infection (i.e., clearing the microbe), wherein the microbial burden is caused by a fungal infection; wherein the fungal infection is an infection with a fungi including, but not limited to Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, and Alternaria alternata.

In one aspect, disclosed herein are methods of reducing, decreasing, removing, clearing, and/or eliminating a microbial infection and/or the microbial burden of the microbial infection (i.e., clearing the microbe), wherein the microbial burden is caused by a parasitic or protozoan infection; and wherein the parasitic or protozoan infection is an infection with a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

In one aspect, it is understood and herein contemplated that despite being able to reduce inflammation and clear microbes from host tissue, organs, or systems without administration of an anti-microbial agent, the disclosed NTCI (such as, for example, SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41) are not microbicidal. Thus, there can also be situations where the addition of an anti-microbial agent to the treatment regimen such as a component in the composition comprising the NTCI or via a separate administration is desired. Accordingly, disclosed herein are methods of reducing, decreasing, removing, clearing, and/or eliminating a microbial infection and/or the microbial burden of the microbial infection in a subject, comprising administering to the subject an anti-microbial agent and a composition comprising one or more NTCI (such as, for example, SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41). The NTCI and anti-microbial agent can be administered as part of the same composition or separately. Examples of anti-microbial agents include but are not limited to antibiotics, antibodies, small molecules, and functional nucleic acids (siRNA, RNAi, anti-sense oligonucleotides), that directly attack the infecting microbe or alter host conditions rendering the host system inhospitable to the microbe.

As noted above anti-microbial agents can comprise any antibiotics, antibodies, small molecules, and functional nucleic acids (siRNA, RNAi, anti-sense oligonucleotides), that directly attack the infecting microbe or alter host conditions rendering the host system inhospitable to the microbe. Such agents include, but are not limited to Abacavir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Lamivudine, Lopinavir, Loviride, Maraviroc, Moroxydine, Methisazone, Nelfinavir, Nevirapine, Nexavir, Nitazoxanide, Norvir, Oseltamivir, Peginterferon alfa-2a, Penciclovir, Peramivir, Pleconaril, Podophyllotoxin, Raltegravir, Ribavirin, Rimantadine, Ritonavir, Pyramidine, Saquinavir, Sofosbuvir, Stavudine, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine, Clofazimine; Dapsone; Capreomycin; Cycloserine; Ethambutol (Bs); Ethionamide; Isoniazid; Pyrazinamide; Rifampicin; Rifabutin; Rifapentine; Streptomycin; Arsphenamine; Chloramphenicol (Bs); Fosfomycin; Fusidic acid; Metronidazole; Mupirocin; Platensimycin; Quinupristin/Dalfopristin; Thiamphenicol; Tigecycline (Bs); Tinidazole; Trimethoprim (Bs); aminoglycosides such as, for example, Amikacin, Gentamicin, Kanamycin, Meropenem, Neomycin, Netilmicin, Tobramycin, Paromomycin, Streptomycin, Spectinomycin, Nitazoxanide, Melarsoprol Eflornithine, Metronidazole, Tinidazole, Miltefosine, Mebendazole, Pyrantel pamoate, Thiabendazole, Diethylcarbamazine, Ivermectin, Niclosamide, Praziquantel, Albendazole, Praziquantel, Rifampin, Amphotericin B, Fumagillin, Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Efinaconazole, Epoxiconazole, Fluconazole, Isavuconazole, Itraconazole, Posaconazole, Propiconazole, Ravuconazole, Terconazole, Voriconazole, Abafungin, Anidulafungin, Caspofungin, Micafungin, Aurones, Benzoic acid, Ciclopirox, Flucytosine, Griseofulvin, Haloprogin, Tolnaftate, Undecylenic acid, Crystal violet, Balsam of Peru, Orotomide, Miltefosine; ansamycins, such as, for example, geldanamycin, rifaximin, herbimycin; Carbapenems, such as, for example, Ertapenem, Doripenem, Imipenem/Cilastatin, and Meropenem; Cephalosporins, such as, for example, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, and Ceftobiprole; Glycopeptides, such as, for example Teicoplanin, Vancomycin, Telavancin, Dalbavancin, and Oritavancin; Lincosamides (Bs), such as, for example, Clindamycin and Lincomycin; Lipopeptides, such as, for example, Daptomycin; Macrolides (Bs), such as, for example, Azithromycin, Clarithromycin, Erythromycin, Roxithromycin, Telithromycin, and Spiramycin; Monobactams, such as, for example, Aztreonam; Nitrofurans, such as, for example, Furazolidone and Nitrofurantoin (Bs); Oxazolidinones (Bs), such as, for example, Linezolid, Posizolid, Radezolid, and Torezolid; Penicillins, such as, for example, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin; Polypeptides, such as, for example, Bacitracin, Colistin, and Polymyxin B; Quinolones/Fluoroquinolones, such as, for example, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin; Sulfonamides (Bs), such as, for example, Mafenide, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Trimethoprim-Sulfamethoxazole (Co-trimoxazole) (TMP-SMX), and Sulfonamidochrysoidine (archaic); Tetracyclines (Bs), such as, for example, Demeclocycline, Doxycycline, Metacycline, Minocycline, Oxytetracycline, and Tetracycline; monoclonal antibodies such as, for example, Actoxumab, Atidortoxumab, Berlimatoxumab, Bezlotoxumab, Cosfroviximab, Edobacomab, Felvizumab, Firivumab, Foravirumab, Larcaviximab, Motavizumab, Navivumab, Panobacumab, Palivizumab, Porgaviximab, CR6261, Rafivirumab, Pagibaximab, Obiltoxaximab, Ibalizumab, Regavirumab, Rmab, Sevirumab, Rivabazumab pegol, Tefibazumab, Suvratoxumab, and Tuvirumab; and checkpoint inhibitors; Pembrolizumab, Nivolumab, Atezolizumab, Avelumab, Durvalumab, pidilizumab, AMP-224, AMP-514, PDR001, cemiplimab, and Ipilimumab.

### 1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### 2. Peptides

### a) Protein Variants

As discussed herein there are numerous variants of the NTCI that are known and herein contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 9 and 10 and are referred to as conservative substitutions.

TABLE 9
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 10
Amino Acid Substitutions
Original Residue Exemplary Conservative Substitutions,
others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 10, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 2 sets forth a particular sequence of cSN50.1. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. MOL Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 9 and Table 10. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. 1 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### a) Materials and Methods

### (1) Synthesis and Purification of the Cell-Penetrating Nuclear Transport Checkpoint Inhibitor (NTCI)

The cell-penetrating NTCI peptide, cSN50.1 (AAVALLPAVLLALLAPCVQRKRQKLMPC)(SEQ ID NO: 2) and its FITC-labeled derivative (AAVALLPAVLLALLAGGK(FITC)GGPCVQRKRQKLMPC) (SEQ ID NO: 42) were synthesized as described elsewhere. Briefly, the peptide chain was assembled through Solid Phase Peptide Synthesis (SPPS) according to standard Fmoc chemistry protocols using an automated peptide synthesizer FOCUS XC (AAPPTec, Louisville, KY). Crude peptides were removed from the resin with a TFA cleavage cocktail and purified by dialysis against double-distilled water in 1 kDa membrane (Spectra/Por 7; Spectrum Laboratories, Rancho Dominguez, CA). The purity and structure of the final products were verified respectively by an analytical C18 RP HPLC (Beckman Coulter GOLD System, Brea, CA) and MALDI mass spectroscopy (Voyager Elite; PerSeptive Biosystems, Framingham, MA). Before treatment, NTCIs (cSN50.1 and FITC-cSN50.1 peptides) were solubilized in sterile water (half the final volume) and diluted with sterile saline to a final concentration of 3.3 mg/ml (cSN50.1) and 4.1 mg/ml (FITC-cSN50.1).

### (2) Preparation of Cecal Microbiome (CM) Stock

8-10-week-old C57BL/6 mice (Jackson Laboratories, Bar Harbor, ME) were euthanized by over inhalation of isoflurane followed by cervical dislocation. The cecum was removed, and the gut microbiome content was extruded into a separate pre-weighed vial according to the published method modified as follows. The collected cecal contents were weighed, and mixed with 5% dextrose (D5W) to final concentration of 80 mg/ml. The cecal content was strained through a 70 μm mesh strainer (VWR, Radnor, PA). The filtered cecal microbiome (~250 μl/vial) was frozen at −80° C. as CM stock. To determine the stock's bacterial count (CFU/ml), 20 μl of fresh CM stock from each tube was diluted 1:100 with sterile PBS, and 100 μl of diluted stock mixture was cultured on Tryptic Soy Agar (TSA)+5% sheep blood plates (Thermo Fisher Scientific, Hampton, NH) and incubated overnight at 37° C. for the count of non-viridans bacterial colonies. For experimental consistency, the tubes with bacterial count averaging between 4.0×10⁴ and 5.0×10⁴ CFU/ml were kept stocked. Before each intraperitoneal injection, several vials were combined to suffice injection of 4.5×10⁵ CFUs/kg. In parallel, a sample of 20 μl from pooled CM stock was tested for quality control, as described above.

### (3) Animal Studies

Animal experiments were carried out in compliance with the ARRIVE guidelines and in strict conformity with the Guide for the Care and Use of Laboratory Animals of the National Institutes of Health. The submitted protocols were approved by the Vanderbilt University Institutional Animal Care and Use Committee. In all animal assays, an adapted model of nonsurgical polymicrobial peritonitis was used. During the course of experiments, mice were closely monitored and euthanized by isoflurane over inhalation followed by cervical dislocation upon expression of the signs of moribund state. Survivors were euthanized at the experimental end point. The experimental groups were selected using a double blinded randomization method. Each experiment was performed at least twice to assure statistical significance and experimental reproducibility. Blood was collected from retroorbital plexus under isoflurane anesthesia shortly before euthanasia.

### (a) Infection and Treatment

The experimental groups (Sham Control+Saline, n=5; CM-Challenged+Saline, n=5; and CM-Challenged+NTCI, n=5) were comprised of randomly assigned 8-10-week-old female C57Bl/6J mice (The Jackson Laboratory, Bar Harbor, ME). The mice were infected by single intraperitoneal (i.p.) injection of a CM stock corresponding to 4.5×10⁵ CFU/kg (between 9-11.5 μl/g of CM stock). For NGS, qRT PCR, and cytokines analyses, mice were treated with the i.p. injection of NTCI (cSN50.1 peptide, 33 μg/g/injection in 200 μl 0.45% NaCl) or 0.45% NaCl (200 μl) as vehicle control, at 30 min before and 30 min, 1.5h, 2.5h, 3.5h, and 5 h after CM challenge. To determine an effect of NTCI treatment on gene expression in lungs and kidneys, mice were treated with NTCI without the CM challenge or vehicle control, following the same injection schedule. For determination of bacterial dissemination in the blood, lungs, spleen, and kidneys, the NTCI and saline treatments were adjusted as follows: 30 min before and 30 min, 1.5h, 2.5h, 3.5h, 6h, and 9 h after CM challenge. Mice were euthanized at 6h for genomic analysis and at 12 h post-CM challenge for bacterial dissemination study.

### (b) Determination of LD₅₀ for Cecal Microbiome (CM)

Randomly grouped (n=6) 8-10-week-old female C57Bl/6J mice (The Jackson Laboratory, Bar Harbor, ME) were infected by single i.p. injection of a CM stock corresponding to 7.5×10⁵, 9.0×10⁵, 1.05×10⁶ or 1.2×10⁶ CFU/kg. Mice were closely monitored throughout the experiment and euthanized upon expression of the signs of moribund state. Survivors were euthanized at 168 h post CM challenge.

### (4) Gene Expression Profiling by qRT PCR

Mice were euthanized at 6 hrs. post CM challenge and isolated lungs were disrupted in lysis buffer on ice using a Dounce hand homogenizer. Total RNA was isolated using NucleoSpin RNA Plus kit (Macherey-Nagel, Germany) according to the manufacturer's instructions. RNA concentration and purity were determined using a NanoDrop One spectrophotometer (Thermo Fisher Scientific, Hampton, NH). The 1 μg of total RNA was reverse-transcribed using iScript cDNA synthesis kit (Bio-Rad, Hercules, CA). A real-time quantitative reverse transcription PCR was carried out in a 96-well plate on a QuantStudio 3 instrument (Thermo Fisher Scientific, Hampton, NH) using Taqman Fast Advanced Master Mix (Thermo Fisher Scientific, Hampton, NH), according to the manufacturer's protocol. FAM-labeled probes for mouse genes were obtained from Thermo Fisher Scientific. The raw Ct values were converted into relative expression levels using Litvak methods (2^{-ΔΔCt}) with 18S rRNA gene as reference and Mock Control (unchallenged and not treated mice) as calibrator (control). Converted Ct values were used for statistical analysis.

### (5) Next-Generation Sequencing (NGS)

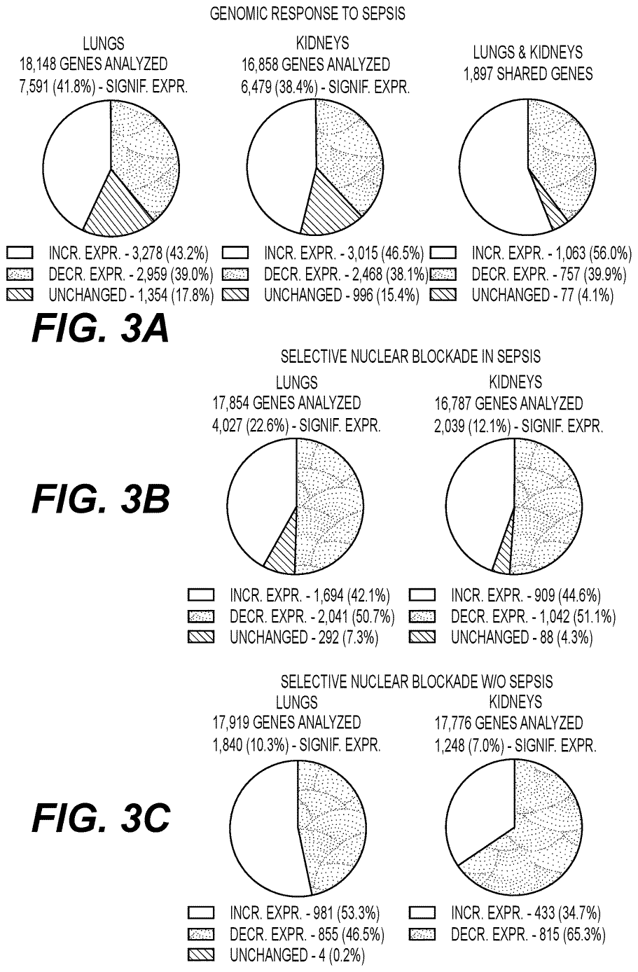

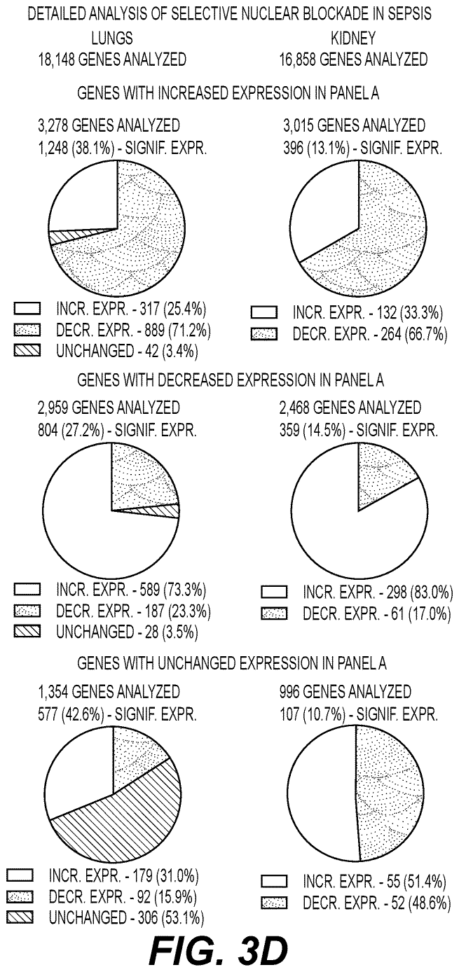

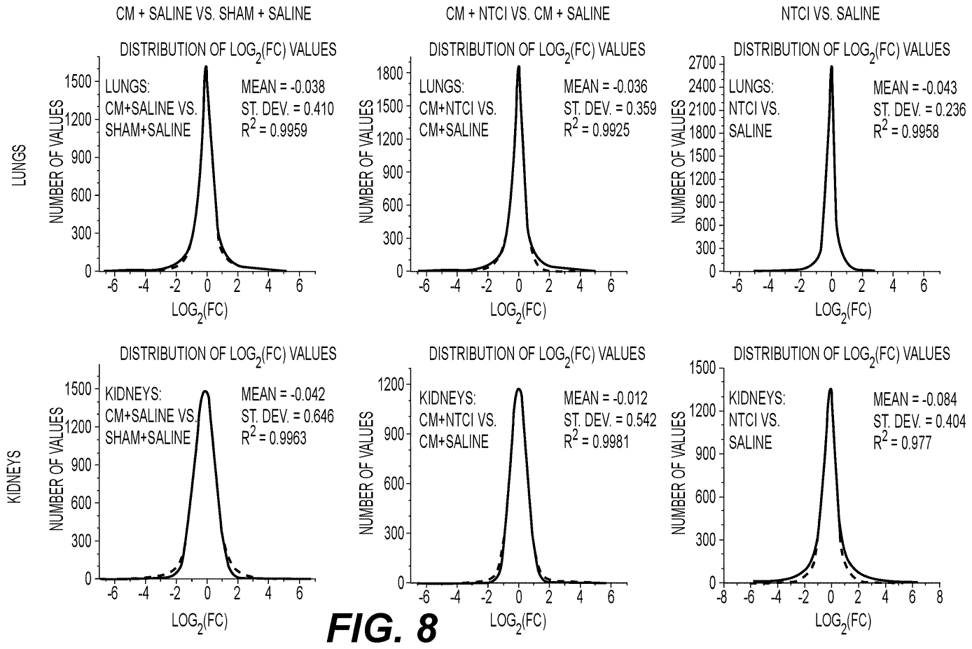

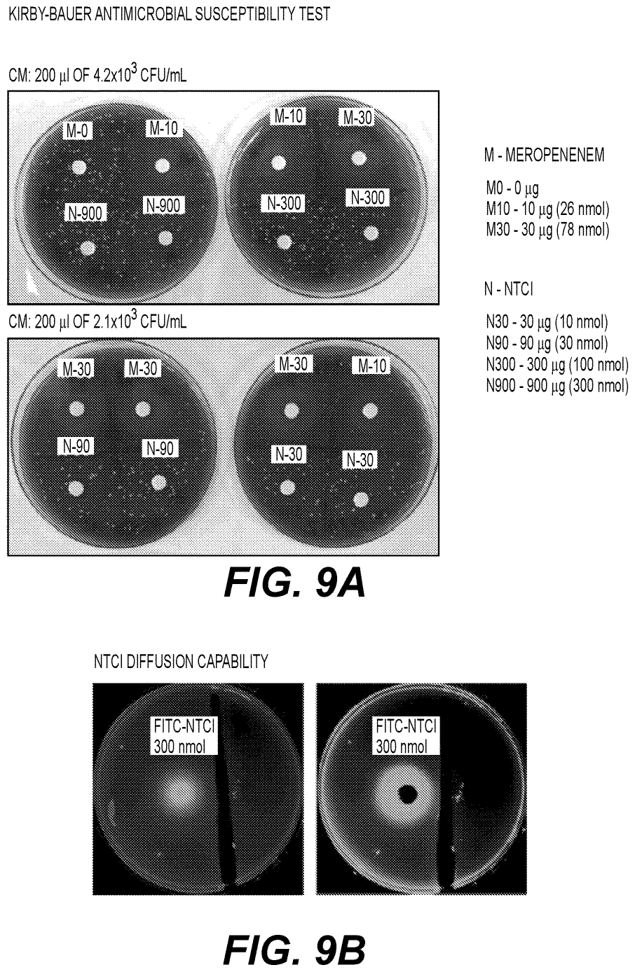

Mice were euthanized at 6 hrs. post CM-challenge and the lung and kidney tissues were processed for total RNA extraction as described above. Three samples from each experimental conditions (Sham+Saline, CM+Saline, CM+cSN50.1, and cSN50.1 w/o CM) from lungs and kidneys were submitted to the Vanderbilt Technologies for Advanced Genomics (VANTAGE) Core for RNA sequencing analysis. RNA integrity numbers (RIN) were measured using TapeStation system (Agilent Technologies, Santa Clara, CA) and total RNA was processed into Stranded mRNA (NEB) Library. RNA sequencing was performed using Illumina NovaSeq6000 (San Diego, CA). The quality control of RNA preparation was analyzed by DRAGEN RNA Pipeline (v3.7.5). The differential expression of the genes was analyzed using Illumina DRAGEN Secondary Analysis software. The expressions of approximately 17 to 18 thousand genes were analyzed in each organ and condition (FIGS. 3A, 3B, & 3C). Number of genes considered as significantly expressed were determined by a false discovery rate (FDR) set to p_{adj}<0.05. Genes with increased/decreased expression, as compared to the experimental control group, were selected from the pool of genes significantly expressed displaying values of log₂(fold change) grater/less than center of distribution+standard deviation determined by Gaussian distribution plot (FIG. 8). Genes with log₂(FC) values located within the range established by standard deviation were considered as genes with unchanged expression (FIG. 3).

The analyzed conditions were: CM+Saline vs. Sham+Saline to evaluate the impact of sepsis on gene expression, and CM+NTCI (cSN50.1) vs. CM+Saline to evaluate the impact of selective nuclear blockade on sepsis-induced gene expression. The condition of NTCI vs. Saline were used to determine an effect of NTCI treatment on gene expression in healthy animals without sepsis (FIG. 3C).

### (6) Determination of Bacterial Dissemination in Blood, Lungs, Spleen and Kidneys

Mice were euthanized 12 hours post CM challenge and blood, spleen, lungs, and kidneys were collected for the analysis of bacterial count. Blood (50 μl) was diluted 1:20 with sterile PBS and 200 μl of diluted blood sample was applied to TSA+5% sheep blood agar culture dish and incubated overnight at 37° C. Isolated organs were externally sterilized by brief immersion in 70% ethanol, washed with cold sterile PBS, and placed in pre-weighed vials containing 0.5 ml cold sterile PBS. Tissue samples were weighed, and net weights were recorded. Organs were homogenized with a disposable sterile plastic homogenizer and suspension passed through the 70 μm cell strainers. 50 μl of suspension was serially diluted with sterile PBS resulting in the final concentration of 1:100 (lungs), 1:10,000 (spleen), and 1:500 (kidneys). The diluted samples (200 μl) were applied to TSA+5% sheep blood agar plate for overnight incubation at 37° C. The colonies (non-viridans) were counted down and then converted into CFU/ml (blood) or CFU/g of a wet organ mass (lungs, spleen, and kidneys).

### (7) Cytokine Assay

The proteins levels of cytokines IL-6, IL-10, TNF-α, and IFN-γ were measured in blood plasma collected postmortem from the hearts of mice euthanized at 6 hrs. post CM/Sham challenge (groups Sham+Saline, CM+Saline, and CM+cSN50.1) or 1 hr. after last NTCI treatment (cSN50.1 only). A cytometric bead array (BD Biosciences) assay was performed and analyzed in the Vanderbilt University Medical Center Flow Cytometry Shared Resource.

### (8) Kirby-Bauer Antimicrobial Susceptibility Test (AST)

To determine the NTCI's antibacterial activity, the indicated amounts of peptide (in 10 μl aliquots) were loaded up to the blank sterile discs (Thermo Scientific™ Oxoid™, Thermo Fisher Scientific, Hampton, NH) using 1 mM or 5 mM stock solution. To allow solvent evaporation, disks, under sterile conditions, were flipped over several times between loads until completely dry. Kirby-Bauer AST was performed using meropenem-loaded disks (10 μg and 30 μg) as positive control. CM was diluted 1:10 and 1:20 with PBS and 200 μl inoculum suspension was spread evenly on TSA+5% sheep blood agar plates. NTCI- or meropenem-loaded discs were placed on inoculated agar plates and pressed gently down to ensure contact with the agar surface. The plates were placed in an incubator set to 37° C. After 24 hours of incubation plates were examined by measuring the diameters of the complete inhibition zones.

To determine the NTCI's diffusion zone from the disks into agar medium, a 300 nmol of FITC-labeled NTCI was loaded on the blank disc. Disc was applied to the surface of the regular agar plate for overnight incubation at 37° C. FITC-labeled NTCI diffusion into the agar gel was determined by fluorescent imaging (Gel Doc EZ imaging system; Bio-Rad, Hercules, CA).

### (9) Statistical Analysis

Normal distribution of data sets was verified using normal probability plot (q-q) and Kolmogorov-Smirnov Normality Test. A statistical analysis was performed using Prism 6 software (GraphPad, Boston, MA). Gene expression profile by qRT PCR of lungs total RNA samples and plasma levels of cytokines were analyzed by ordinary one-way ANOVA with an uncorrected Fisher's LSD test for a multiple comparison. Bacterial dissemination in blood, lungs, spleen and kidneys was analyzed by nonparametric t test with Mann-Whitney rank comparison. The data is presented as a mean±SEM. p values of <0.05 were considered significant. The standard deviation and the center of the log₂(FC) values distribution in NGS analyzed data was determined by the Gaussian plot (FIG. 8). Log₂(FC) values were rounded down to the nearest decimal point (0.1) and equal values were added up. These numbers were then plotted against log₂(FC) and analyzed by a Gaussian/Lorentzian function using Prism 6 software (GraphPad, Boston, MA).

### b) Results

(1) Sepsis Induces the Genes Encoding Mediators of Inflammation in the Lungs while the Selective Nuclear Blockade with the NTCI Suppresses this Induction as Detected by the Two Independent Methods [Quantitative PCR and the Next Generation Sequencing (NGS)].

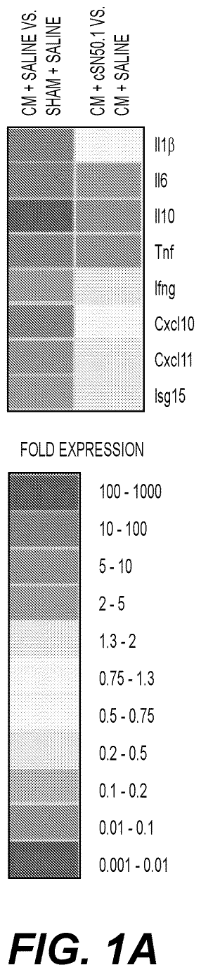

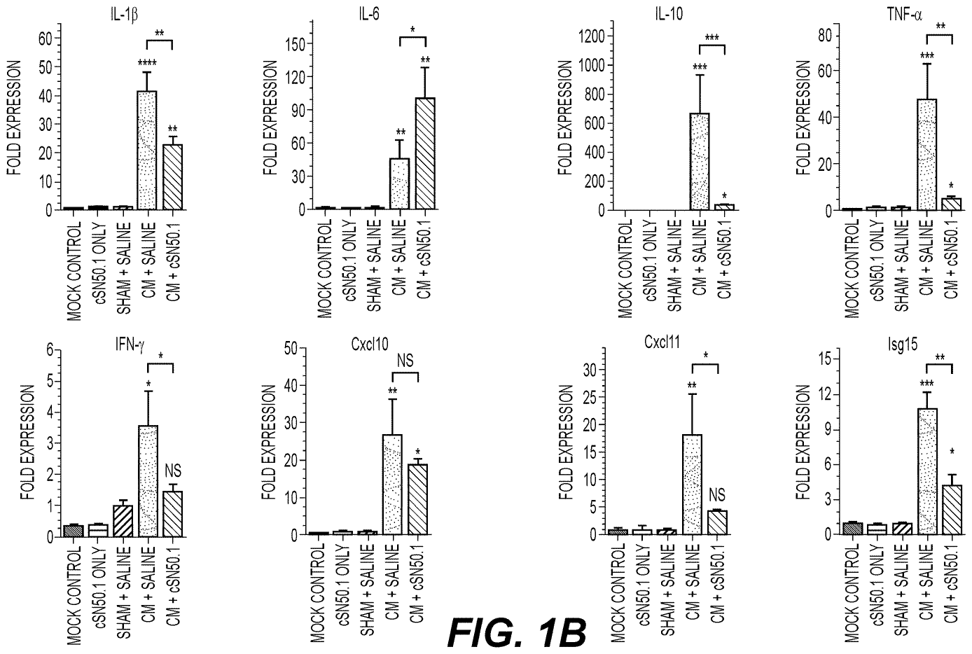

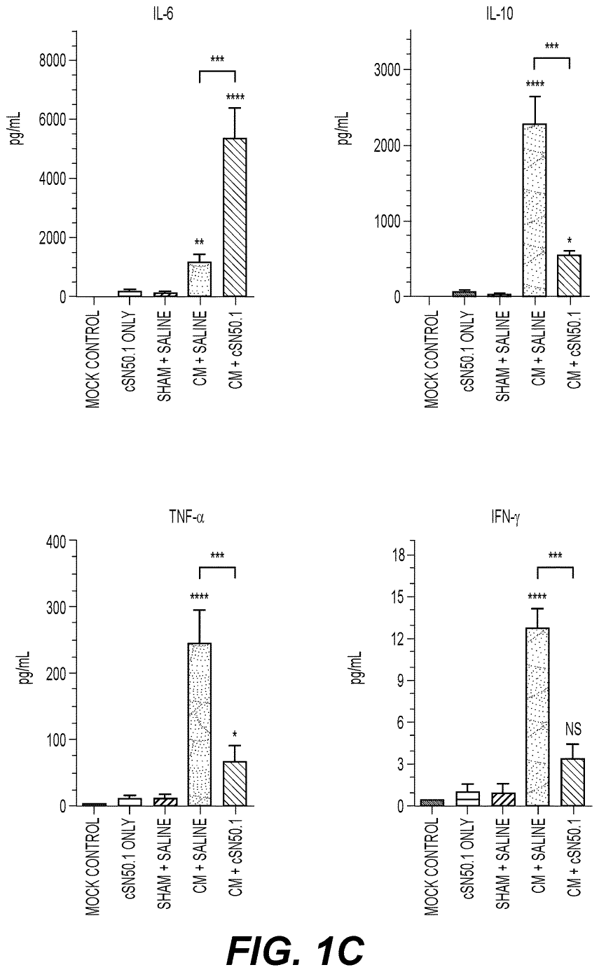

The lungs and kidneys are the major organs that fail during sepsis. Therefore, we focused first on the lungs, the site of ARDS, a severe complication of infections. In the polymicrobial sepsis, we compared the expression of the selected genes encoding mediators of inflammation analyzed by the quantitative PCR, to that determined by the NGS (FIGS. 1A and 1C). The 8 representative genes encoding cytokines and chemokines (IL-1B, IL-6, IL-10, TNFα, IFN-γ, CXCL10, CXCL11), and ISG15 (interferon-stimulated gene 15, which encodes the ubiquitin-like modifier of intracellular signaling proteins), were consistently induced in the lungs during the experimental sepsis. The NTCI (cSN50.1 peptide) suppressed their expression, as analyzed by both genomic methods (FIGS. 1A and 1B). One notable exception was the gene encoding the pleiotropic cytokine, IL-6, which remained elevated in the NTCI-treated mice. The consistency of our results based on the two methods of genomic analysis was further supported by the proteomic analysis of the corresponding changes in the blood plasma proteins encoded by the selected genes (FIG. 1C). As expected, the protein levels of IL-10, TNFα, and IFN-γ were increased in the CM-challenged mice. The treatment with NTCI (cSN50.1 peptide) significantly reduced their concentration reflecting their cognate gene suppression in the lungs (FIGS. 1A and 1B). Contrariwise, the IL-6 protein level remained elevated in the plasma of the NTCI-treated animals (FIG. 1C). Together, the concordance of the two methods of genomic analysis was also consistent with the proteomic analysis of plasma proteins. Reassured by these results, we embarked on the comparative study of the genomic response to sepsis in the two major target organs, lungs and kidneys, using the NGS.

(2) the Sepsis-Induced Genes Encoding Mediators of Inflammation in the Lungs and Kidneys are Controlled by the Selective Nuclear Blockade with the NTCI.

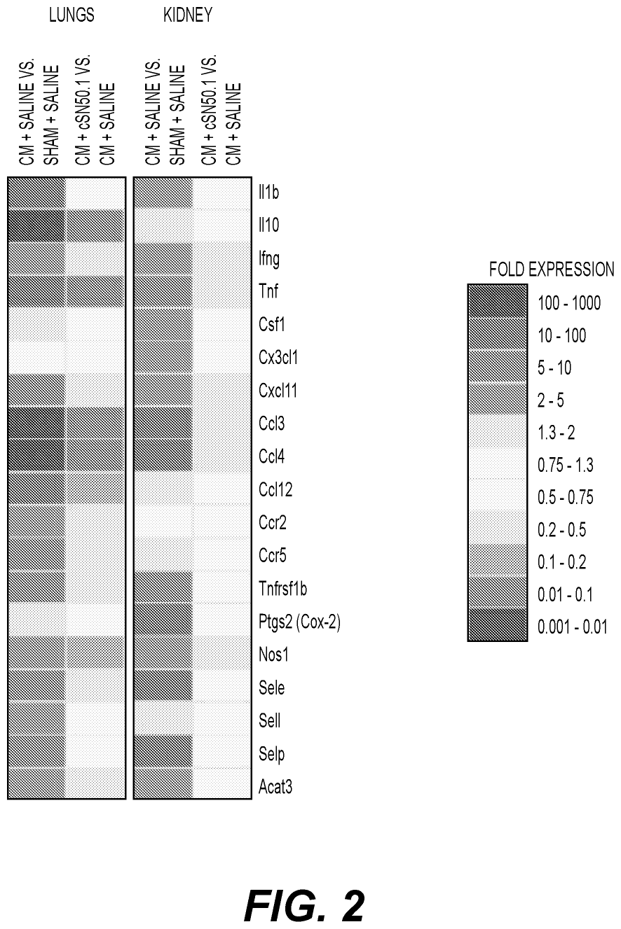

The first group of the 19 genes encoding the known mediators of inflammation in the lungs and kidneys included the following: (i) genes expressing cytokines: Il1b, Tnf, Ifng, Il10; (ii) chemokines: Ccl3, Ccl4, Ccl12, Ccr2,Ccr5; (iii) hemopoietic growth factor (Csf1), also known as granulocyte/monocyte colony-stimulating factor; (iv) cell adhesion molecules: selectins (endothelial, platelet, and leucocyte); and (v) genes encoding signal transducers: cyclooxygenase-2, nitric oxide synthase-1, and cholesterol acyltransferase 3 (Acat3), the catalyst in the cholesterol esters synthesis. As shown in FIG. 2, among these 19 representative genes in the lungs and kidneys, 12 displayed very similar pattern of expression in both organs whereas Csf1, Cx3cl1, and Ptgs2 (Prostaglandin-endoperoxide synthase 2, aka Cox2) showed higher expression in the kidneys. The treatment with the NTCI dramatically counteracted the activation of these genes in both organs. Remarkably, the six sepsis-elevated genes in the lungs (Il10, Tnf, Ccl3, Ccl4, Ccl12, and Nos1) were more susceptible to the nuclear blockade with the NTCI than their homologs in the kidneys (FIG. 2). The cellular and molecular architecture of the two organs can contribute to their different genomic response to sepsis.

(3) Sepsis Causes Massive Genomic Response in the Lungs and Kidneys that is Countered by the NTCI

The impact of experimental sepsis on the transcriptome in the lungs and kidneys was astounding (FIG. 3). In the lungs, among the 18,148 analyzed genes comprising the lung transcriptome, 7,591 (41.8%) genes were significantly expressed during sepsis. Within this set, 3,278 genes (43.2%) displayed increased expression and 2,959 genes (39%) showed decreased expression whereas 1,354 genes (17.8%) remained unchanged. In the kidneys, among the 16,858 analyzed genes comprising the kidneys transcriptome, 6,479 (38.4%) showed significant expression. Within this set, the expression of 3,015 genes (46.5%) was increased and 2,468 genes (38.1%) was decreased in response to sepsis (FIG. 3A). The expression of 996 (15.4%) genes did not show significant difference from uninfected control. The increased expression of 1,063 genes was observed in both organs whereas the expression of 757 genes was decreased in lungs and kidneys. In total, 1,897 genes shared common pattern of expression in the two organs.

This extraordinary genomic response to sepsis in the two major organs was profoundly changed by the selective nuclear blockade with the NTCI during the 6-hour treatment (FIG. 3B). Among 17,854 genes analyzed in the lungs and 16,787 genes analyzed in the kidneys, 4,027 genes were significantly expressed in the lungs and 2,039 genes in the kidneys. The impact of the NTCI treatment on the gene expression in the lungs and kidneys was bidirectional. In the lungs, 1,694 (42.1%) genes were increased and 2,041 genes (50.7%) were decreased whereas 292 genes (7.3%) were unchanged during selective nuclear blockade with NTCI. In the kidneys, the expression of 909 (44.6%) genes was increased and 1,042 (51.1%) genes were decreased while the expression of 88 (4.3%) genes remained unchanged during selective nuclear blockade with NTCI (FIG. 3B).

We also considered the impact of NTCI on the gene expression in control mice not challenged with sepsis-producing cecal microbiome (CM) (FIG. 3C). The NGS analysis of selective nuclear blockade by NTCI in control animals without sepsis showed that among 10.3% genes significantly expressed in the lungs (1,840 of 17,919 analyzed), 981 (53.3%) showed an upward and 855 (46.5%) displayed downward expression. In the kidneys, where only 7% of analyzed genes were significantly expressed (1,248 of 17,776 analyzed), the majority, 815 genes (65.2%), showed downward regulation, whereas expression of 433 genes (34.7%) was increased (FIG. 3C). Genomic response in animals without sepsis treated with the multiple intraperitoneal injections of the NTCI or saline (control) could be partially attributed to the stress associated with repeated interventions.

The detailed analysis of gene expression in infected animals treated with NTCI (FIG. 3D) showed that selective nuclear blockade with cSN50.1 decreased expression of 889 genes in the pool of genes increased by sepsis in the lungs (Panel A). Likewise, the NTCI treatment reduced expression of 66.7% genes in the pool of genes increased in the kidneys. In contrast, the expression of 589 lung genes and 298 kidney genes that were decreased in sepsis were strikingly increased by the NTCI (73.3% and 83.0%, respectively). These results in the animals with polymicrobial sepsis document the normalizing effect of the NTCI on genomic response to the microbial inducers and the host-produced mediators of inflammation.

Thus, we found that experimental sepsis elicits the enormous mobilization of the genome in the lungs and kidneys. The virulence factors of sepsis-causing microbial agents together with the host-produced mediators of inflammation, massively activated inflammatory and metabolic regulome in the cell nuclei of the lungs and kidneys. Our findings of this unprecedented “genomic storm” in the lungs and kidneys advance the understanding of sepsis through the identification of the genes displaying common pattern of expression, shared by both organs, and the genes displaying organ-specific expression in the lungs and kidneys involved in response to sepsis. Moreover, we expanded this analysis to the genes controlled by the selective nuclear blockade with the NTCI in the lungs and kidneys of infected animals.

(4) the Top 100 Shared Genes Responding to Sepsis in the Lungs and Kidneys that are Regulated by the NTCI.

The roster of the top 100 genes changed by sepsis in the lungs and kidneys as compared to the genes analyzed in the unchallenged “sham” controls treated with saline is presented in the Table 1. The 11 most sepsis-upregulated genes in the lungs differed in their maximal response from that of the kidneys. Nevertheless, there was a similar pattern of their inhibition by the NTCI with the notable exception of the gene encoding Colony Stimulating Factor 3 (granulocyte, Csf3), which was not suppressed by the NTCI in both organs. The gene encoding Interleukin 10 (IL10) was robustly responding to sepsis in the lungs being reduced almost 20-fold by NTCI albeit the almost 70-fold lower expression in the kidneys was unchanged by NTCI. Similarly, the highly elevated expression of the gene encoding chemokine (C-X-C motif) ligand 2 (Cxcl2) was reduced by the NTCI in the lungs but increased in the kidneys.

TABLE 1
Genes with Increased Expression in Sepsis Shared by Lungs and Kidneys

| | | | Lungs | | Kidneys | |
|---|---|---|---|---|---|---|
| | Gene ID | Gene Name | Saline | NTCI | \|Saline | NTCI |
| 1 | Csf3 | colony stimulating factor 3 (granulocyte) | 1,347 | 1,296 | 847 | 1,168 |
| 2 | Il10 | interleukin 10 | 460 | 25 | 6 | 7 |
| 3 | Acod1 | aconitate decarboxylase 1 | 452 | 103 | 73 | 53 |
| 4 | Saa3 | serum amyloid A 3 | 412 | 106 | 23 | 12 |
| 5 | Cxcl2 | chemokine (C-X-C motif) ligand 2 | 323 | 215 | 217 | 555 |
| 6 | Gpr31b | G protein-coupled receptor 31, D17Leh66b region | 306 | 67 | 5 | 5 |
| 7 | Fgf23 | fibroblast growth factor 23 | 227 | 437 | 28 | 27 |
| 8 | Igfbp1 | insulin-like growth factor binding protein 1 | 111 | 80 | 5 | 8 |
| 9 | Mt2 | metallothionein 2 | 106 | 123 | 14 | 39 |
| 10 | Mrgpra2b | MAS-related GPR, member A2B | 103 | 78 | 29 | 71 |
| 11 | Ccl3 | chemokine (C-C motif) ligand 3 | 101 | 9 | 12 | 4 |
| 12 | Ccl4 | chemokine (C-C motif) ligand 4 | 98 | 3 | 12 | 3 |
| 13 | Gpr84 | G protein-coupled receptor 84 | 81 | 19 | 20 | 17 |
| 14 | Adamts4 | a disintegrin-like and metallopeptidase (reprolysin type) with | 78 | 86 | 27 | 16 |
| | | thrombospondin type 1 motif, 4 | | | | |
| 15 | Madcam1 | mucosal vascular addressin cell adhesion molecule 1 | 76 | 46 | 47 | 20 |
| 16 | Il1r2 | interleukin 1 receptor, type II | 73 | 145 | 8 | 32 |
| 17 | Olfm4 | olfactomedin 4 | 71 | 31 | 2 | 2 |
| 18 | Il1f9 | interleukin 36G | 68 | 9 | 34 | 16 |
| 19 | Cxcl1 | chemokine (C-X-C motif) ligand 1 | 64 | 61 | 123 | 262 |
| 20 | Gpr31a | G protein-coupled receptor 31, D17Leh66a region | 62 | 16 | 12 | 4 |
| 21 | Gpr31c | G protein-coupled receptor 31, D17Leh66c region | 60 | 17 | 3 | 2 |
| 22 | Ptx3 | pentraxin related gene | 57 | 47 | 137 | 227 |
| 23 | Slc7a11 | solute carrier family 7 (cationic amino acid transporter, y+ | 57 | 30 | 6 | 12 |
| | | system), member 11 | | | | |
| 24 | Gm6553 | G protein-coupled receptor 31, D17Leh66c region pseudogene | 55 | 17 | 14 | 10 |
| 25 | Tmprss11g | transmembrane protease, serine 11g | 54 | 103 | 18 | 39 |
| 26 | Tac1 | tachykinin 1 | 54 | 80 | 27 | 86 |
| 27 | Il1f6 | interleukin 36A | 51 | 4 | 64 | 114 |
| 28 | Ccl2 | chemokine (C-C motif) ligand 2 | 50 | 16 | 16 | 17 |
| 29 | Psg16 | pregnancy specific beta-1-glycoprotein 16 | 48 | 39 | 10 | 6 |
| 30 | Fosl1 | fos-like antigen 1 | 48 | 31 | 11 | 24 |
| 31 | Sprr2a2 | small proline-rich protein 2A2 | 45 | 51 | 2 | 7 |
| 32 | Serpina3m | serine (or cysteine) peptidase inhibitor, clade A, member 3M | 45 | 29 | 17 | 7 |
| 33 | Il1rn | interleukin 1 receptor antagonist | 43 | 14 | 165 | 117 |
| 34 | Cxcl3 | chemokine (C-X-C motif) ligand 3 | 42 | 103 | 207 | 421 |
| 35 | Ccl20 | chemokine (C-C motif) ligand 20 | 41 | 41 | 8 | 8 |
| 36 | Tnf | tumor necrosis factor | 38 | 4 | 9 | 6 |
| 37 | Gm43181 | predicted gene 43181 | 37 | 13 | 23 | 15 |
| 38 | Clec4e | C-type lectin domain family 4, member e | 37 | 18 | 45 | 83 |
| 39 | Gm45774 | predicted gene 45774 | 35 | 22 | 7 | 4 |
| 40 | Ugt1a10 | UDP glycosyltransferase 1 family, polypeptide A10 | 35 | 39 | 4 | 2 |
| 41 | Kcnt1 | potassium channel, subfamily T, member 1 | 32 | 28 | 2 | 2 |
| 42 | Mt1 | metallothionein 1 | 31 | 39 | 6 | 19 |
| 43 | Mefv | Mediterranean fever | 31 | 20 | 17 | 18 |
| 44 | Clec4d | C-type lectin domain family 4, member d | 31 | 16 | 12 | 13 |
| 45 | Ms4a4a | membrane-spanning 4-domains, subfamily A, member 4A | 31 | 12 | 12 | 7 |
| 46 | Btbd16 | BTB (POZ) domain containing 16 | 30 | 10 | 32 | 18 |
| 47 | Spic | Spi-C transcription factor (Spi-1/PU.1 related) | 29 | 13 | 3 | 4 |
| 48 | Orm1 | orosomucoid 1 | 29 | 10 | 8 | 3 |
| 49 | Serpina3f | serine (or cysteine) peptidase inhibitor, clade A, member 3F | 28 | 15 | 76 | 86 |
| 50 | Il1b | interleukin 1 beta | 27 | 16 | 5 | 5 |
| 51 | Serpina10 | serine (or cysteine) peptidase inhibitor, clade A (alpha-1 | 27 | 9 | 6 | 2 |
| | | antiproteinase, antitrypsin), member 10 | | | | |
| 52 | 9830107B12Ri | RIKEN cDNA 9830107B12 gene | 27 | 9 | 22 | 4 |
| | k | | | | | |
| 53 | Gm49839 | predicted gene, 49839 | 27 | 43 | 10 | 16 |
| 54 | Msr1 | macrophage scavenger receptor 1 | 27 | 7 | 6 | 6 |
| 55 | Ankrd2 | ankyrin repeat domain 2 (stretch responsive muscle) | 26 | 14 | 13 | 15 |
| 56 | Saa4 | serum amyloid A 4 | 25 | 5 | 14 | 1 |
| 57 | S100a8 | S100 calcium binding protein A8 (calgranulin A) | 25 | 14 | 15 | 21 |
| 58 | Hpx | hemopexin | 24 | 7 | 25 | 1 |
| 59 | Apoa4 | apolipoprotein A-IV | 24 | 2 | 2 | 2 |
| 60 | Steap4 | STEAP family member 4 | 23 | 15 | 39 | 17 |
| 61 | Cxcl10 | chemokine (C-X-C motif) ligand 10 | 22 | 12 | 9 | 8 |
| 62 | S100a9 | S100 calcium binding protein A9 (calgranulin B) | 22 | 15 | 14 | 21 |
| 63 | Wfdc17 | WAP four-disulfide core domain 17 | 22 | 5 | 4 | 3 |
| 64 | Mmp8 | matrix metallopeptidase 8 | 22 | 19 | 233 | 282 |
| 65 | Gnat1 | G protein subunit alpha transducin 1 | 22 | 17 | 6 | 19 |
| 66 | Fgg | fibrinogen gamma chain | 21 | 7 | 36 | 19 |
| 67 | Fgb | fibrinogen beta chain | 20 | 6 | 26 | 2 |
| 68 | Lilr4b | leukocyte immunoglobulin-like receptor, subfamily B, member 4B | 20 | 13 | 4 | 6 |
| 69 | 2010003K11Ri | RIKEN cDNA 2010003K11 gene | 20 | 6 | 6 | 9 |
| | k | | | | | |
| 70 | Serpina3n | serine (or cysteine) peptidase inhibitor, clade A, member 3N | 20 | 11 | 29 | 9 |
| 71 | AA467197 | expressed sequence AA467197 | 19 | 14 | 39 | 40 |
| 72 | Gm16685 | predicted gene, 16685 | 19 | 19 | 4 | 3 |
| 73 | Timp1 | tissue inhibitor of metalloproteinase 1 | 19 | 14 | 7 | 6 |
| 74 | Fga | fibrinogen alpha chain | 18 | 6 | 37 | 24 |
| 75 | Ifitm6 | interferon induced transmembrane protein 6 | 18 | 19 | 7 | 14 |
| 76 | Lcn2 | lipocalin 2 | 18 | 9 | 53 | 53 |
| 77 | F10 | coagulation factor X | 18 | 3 | 5 | 1 |
| 78 | Leap2 | liver-expressed antimicrobial peptide 2 | 17 | 4 | 6 | 1 |
| 79 | 116 | interleukin 6 | 17 | 56 | 223 | 671 |
| 80 | Retnlg | resistin like gamma | 16 | 16 | 9 | 22 |
| 81 | Ccl7 | chemokine (C-C motif) ligand 7 | 16 | 7 | 13 | 19 |
| 82 | Ikbke | inhibitor of kappaB kinase epsilon | 15 | 4 | 3 | 1 |
| 83 | Vcan | versican | 14 | 8 | 7 | 9 |
| 84 | F13a1 | coagulation factor XIII, A1 subunit | 14 | 6 | 3 | 2 |
| 85 | Serpina3g | serine (or cysteine) peptidase inhibitor, clade A, member 3G | 14 | 9 | 14 | 11 |
| 86 | 1200007C13Ri | RIKEN cDNA 1200007C13 gene | 14 | 19 | 35 | 73 |
| | k | | | | | |
| 87 | Ngp | neutrophilic granule protein | 13 | 13 | 481 | 612 |
| 88 | Siglece | sialic acid binding Ig-like lectin E | 13 | 5 | 3 | 2 |
| 89 | Lrrc25 | leucine rich repeat containing 25 | 13 | 6 | 3 | 3 |
| 90 | Gm10309 | predicted gene 10309 | 13 | 8 | 36 | 20 |
| 91 | Trem1 | triggering receptor expressed on myeloid cells 1 | 13 | 9 | 9 | 15 |
| 92 | Slc39a14 | solute carrier family 39 (zinc transporter), member 14 | 13 | 8 | 5 | 4 |
| 93 | Fgl1 | fibrinogen-like protein 1 | 13 | 4 | 4 | 1 |
| 94 | Il27 | interleukin 27 | 12 | 3 | 8 | 6 |
| 95 | Tcp10b | t-complex protein 10b | 12 | 13 | 5 | 3 |
| 96 | Serpina3i | serine (or cysteine) peptidase inhibitor, clade A, member 31 | 12 | 11 | 26 | 68 |
| 97 | Cxcl9 | chemokine (C-X-C motif) ligand 9 | 12 | 4 | 16 | 16 |
| 98 | A730049H05Ri | RIKEN cDNA A730049H05 gene | 12 | 30 | 2 | 2 |
| | k | | | | | |
| 99 | Gm8883 | predicted gene 8883 | 12 | 1 | 4 | 2 |
| 100 | Fpr2 | formyl peptide receptor 2 | 12 | 9 | 25 | 31 |

Strikingly, the Saa3 gene encoding the acute phase protein response biomarker (Serum Amyloid A-3), was increased by sepsis in the lungs (412-fold) as compared to the kidneys (23-fold) (Table 1). Whereas the role of cytokines and chemokines in sepsis is generally known, the impact of elevated serum amyloid proteins A1-A3, as acute phase proteins akin to the C-Reactive Protein in humans, came to fore recently. The serum amyloid proteins A1-A3 contribute to muscle atrophy, known as the Intensive Care Unit-Acquired Weakness (ICUAW) delaying patients' recovery. Serum Amyloid A1 is recognized by the Toll-like Receptors 2 and 4 triggering nuclear signaling mediated by NF-κB and leading to the myotube injury. Importantly, the treatment with the NTCI lowered Saa3 gene expression four-fold in the lungs and two-fold in the kidneys.

The gene encoding aconitate decarboxylase (Acod1), also known as the immune responsive gene 1 (IRG1) was strikingly induced 452-fold by sepsis in the lungs whereas the NTCI attenuated its expression to the 103-fold level over the untreated control. In comparison, the same gene in the kidneys was induced by sepsis less robustly (73-fold) while the NTCI reduced its expression to 53-fold level. The product of IRG1, itaconate, suppresses succinate dehydrogenase while accumulation of succinate controls adipose tissue thermogenesis. Moreover, itaconate causes the electrophilic stress mobilizing the transcription factors NRF2 and ATF3. Nuclear translocation of NRF2 is mediated by Importin α5 targeted by the NTCI. Thus, NTCI not only suppresses the generation of itaconate but also its electrophilic stress-induced nuclear signaling mediated by NRF2.

Among the analyzed “common genes” shared between the lungs and kidneys is the gene encoding Fos-like antigen (Fosl1), a component of the AP1 transcription factor complex that belongs to the Stress-Responsive Transcription Factors. It was upregulated by sepsis 48-fold and 11-fold in the lungs and kidneys, respectively. The NTCI treatment downregulated its expression to the 31-fold level in the lungs. Contrariwise, expression of Fosl1 gene in the kidneys was increased two-fold by NTCI. Importantly, in addition to changing the expression of Fosl1, the NTCI inhibited nuclear translocation of FOSL1 bound to cJun (AP1 protein complex) during sepsis. FOSL1 was also identified in transcriptomic analysis of differentially expressed genes in SARS-Co-2 infected lung epithelial cells as compared to the pancreatic islet cells.

Genes encoding members of the acute phase protein response, C-reactive protein (Crp), and orosomucoid 1 and 2 (Orm1, Orm2) belong to the group of genes upregulated by sepsis in both organs. The gene encoding Orm1 was tightly regulated by NTCI treatment, especially in the kidneys, where its expression was reduced almost to the physiologic levels. Similarly, three genes encoding fibrinogen alpha (Fga), beta (Fgb), and gamma chains (Fgg), and Coagulation Factor X (F10) were increased by sepsis in the lungs and kidneys. Coagulation Factor X and fibrinogen, the main blood clotting protein, are also elevated during the aforementioned acute phase protein response contributing to the blood hypercoagulability known as thrombophilia. In combination with the microvascular endothelial injury, it evolves into disseminated intravascular coagulation (DIC), one of the major hematologic complications of sepsis. The NTCI treatment reduced the expression of the genes encoding fibrinogen chains and Factor X in the lungs and kidneys thereby reducing the risk of DIC in sepsis, and by extension, in thrombophilia. Consistent with the current findings, the NTCI reduced the signs of massive microvascular liver injury and the laboratory signs of DIC.

We also spotted the genes that are not controlled by the NTCI treatment among the top responders to sepsis in the lungs and kidneys. Besides Csf3, this group encompasses metallothionein 2 (Mt2), interleukin 1 receptor type II (Il1r2), neutrophilic granule protein (Ngp), matrix metallopeptidase 8 (Mmp8), interleukin 6 (Il6), chemokine (C-X-C motif) ligand 1, 2 and 3 (Cxcl1, Cxcl2, Cxcl3), and CD177 antigen (Cd177).

The most downregulated genes by sepsis in the lungs and kidneys feature anoctamin 5 encoding gene Ano5 (Table 2). Its product comprises a 913-amino acid protein of the anoctamin family that is involved in phospholipid scrambling required for the sarcolemma repairing process in the skeletal muscles. Mutations in Ano5 give rise to an autosomal dominant skeletal muscle myopathies and dystrophies. It is likely that the suppression of anoctamin 5 gene in sepsis can contribute to the aforementioned Intensive Care Unit-Acquired Weakness (ICUAW) linked to the elevated Amyloid A-1-3 in sepsis. NTCI treatment raised 5-fold the expression of Anoctamin 5 gene while reducing significantly Amyloid 1-3 genes in sepsis (Tables 1 and 3).

TABLE 2
Genes with Decreased Expression in Sepsis Shared by Lungs and Kidneys

| | | | Lungs | | Kidneys | |
|---|---|---|---|---|---|---|
| | Gene ID | Gene Name | Saline | NTCI | Saline | NTC |
| 1 | Ano5 | anoctamin 5 | −53 | −9 | −17 | −11 |
| 2 | Gsg1l | GSG1-like | −31 | −7 | −72 | −126 |
| 3 | Spon2 | spondin 2, extracellular matrix protein | −27 | −13 | −16 | −3 |
| 4 | Oxtr | oxytocin receptor | −26 | −1 | −22 | −14 |
| 5 | Rnf112 | ring finger protein 112 | −21 | −12 | −39 | NS |
| 6 | Ppp2r2c | protein phosphatase 2, regulatory subunit B, gamma | −17 | −5 | −4 | −33 |
| 7 | Crabp1 | cellular retinoic acid binding protein I | −16 | −7 | −30 | −5 |
| 8 | St6galnac3 | ST6 (alpha-N-acetyl-neuraminyl-2,3-beta-galactosyl-1,3)-N- | −15 | −7 | −4 | −3 |
| | | acetylgalactosaminide alpha-2,6-sialyltransferase 3 | | | | |
| 9 | Agtr2 | angiotensin II receptor, type 2 | −12 | −2 | −23 | −39 |
| 10 | Gjc2 | gap junction protein, gamma 2 | −11 | −9 | −23 | −28 |
| 11 | Gm33195 | predicted gene, 33195 | −11 | −1 | −6 | −2 |
| 12 | Gabra4 | gamma-aminobutyric acid (GABA) A receptor, subunit alpha 4 | −10 | −5 | −4 | −5 |
| 13 | Aplnr | apelin receptor | −10 | −4 | −3 | −6 |
| 14 | E030013/19Ri | RIKEN cDNA E030013119 gene | −9 | −7 | −6 | −3 |
| | k | | | | | |
| 15 | Pcdh12 | protocadherin 12 | −9 | −10 | −3 | −4 |
| 16 | Chp2 | calcineurin-like EF hand protein 2 | −9 | −5 | −4 | −3 |
| 17 | Gng8 | guanine nucleotide binding protein (G protein), gamma 8 | −9 | −5 | −20 | −48 |
| 18 | Pla2g2d | phospholipase A2, group IID | −8 | −3 | −5 | −4 |
| 19 | Gm20683 | predicted gene 20683 | −8 | −2 | −2 | −1 |
| 20 | Mansc4 | MANSC domain containing 4 | −8 | −4 | −3 | −2 |
| 21 | Gm10824 | predicted gene 10824 | −8 | −5 | −5 | −5 |
| 22 | Ces2b | carboxyesterase 2B | −8 | −5 | −3 | −1 |
| 23 | Kank4 | KN motif and ankyrin repeat domains 4 | −7 | −12 | −3 | −4 |
| 24 | Zmat4 | zinc finger, matrin type 4 | −7 | −9 | −4 | −2 |
| 25 | Cdr1os | cerebellar degeneration related antigen 1, opposite strand | −7 | −3 | −43 | NS |
| 26 | Wnt2 | wingless-type MMTV integration site family, member 2 | −7 | −6 | −4 | −3 |
| 27 | Gm17040 | predicted gene 17040 | −7 | −1 | −3 | −2 |
| 28 | Wfdc16 | WAP four-disulfide core domain 16 | −7 | −5 | −2 | −1 |
| 29 | Rab42 | RAB42, member RAS oncogene family | −7 | −6 | −5 | −8 |
| 30 | Tbxa2r | thromboxane A2 receptor | −7 | −9 | −4 | −10 |
| 31 | Plppr5 | phospholipid phosphatase related 5 | −7 | −2 | −47 | −6 |
| 32 | Misp3 | MISP family member 3 | −6 | −11 | −3 | −2 |
| 33 | Mmp28 | matrix metallopeptidase 28 (epilysin) | −6 | −6 | −2 | −3 |
| 34 | Lrrn4 | leucine rich repeat neuronal 4 | −6 | −4 | −11 | −6 |
| 35 | Sost | sclerostin | −6 | −9 | −107 | −17 |
| 36 | Tmem200a | transmembrane protein 200A | −6 | −5 | −10 | −6 |
| 37 | Shisa2 | shisa family member 2 | −6 | −11 | −2 | −3 |
| 38 | Stmn2 | stathmin-like 2 | −6 | −2 | −4 | −3 |
| 39 | Chrm1 | cholinergic receptor, muscarinic 1, CNS | −6 | −3 | −7 | −25 |
| 40 | Tmeff2 | transmembrane protein with EGF-like and two follistatin-like | −6 | −3 | −8 | −5 |
| | | domains 2 | | | | |
| 41 | 2810433D01 | RIKEN cDNA 2810433D01 gene | −6 | −7 | −4 | −3 |
| | Rik | | | | | |
| 42 | Pabpc4l | poly(A) binding protein, cytoplasmic 4-like | −6 | −6 | −6 | −18 |
| 43 | Dact3 | dishevelled-binding antagonist of beta-catenin 3 | −6 | −6 | −5 | −7 |
| 44 | Mettl24 | methyltransferase like 24 | −6 | −3 | −3 | −2 |
| 45 | Gpr34 | G protein-coupled receptor 34 | −6 | −5 | −14 | −8 |
| 46 | D430019H16 | RIKEN cDNA D430019H16 gene | −6 | −9 | −7 | −13 |
| | Rik | | | | | |
| 47 | Hoxc5 | homeobox C5 | −6 | −13 | −2 | −4 |
| 48 | Tekt5 | tektin 5 | −5 | −2 | −6 | −6 |
| 49 | Megf10 | multiple EGF-like-domains 10 | −5 | −3 | −34 | −22 |
| 50 | Wscd1 | WSC domain containing 1 | −5 | −4 | −2 | −4 |
| 51 | Gm32219 | predicted gene, 32219 | −5 | −3 | −51 | −33 |
| 52 | Tril | TLR4 interactor with leucine-rich repeats | −5 | −5 | −3 | −5 |
| 53 | Agtr1b | angiotensin II receptor, type 1b | −5 | −3 | −17 | −41 |
| 54 | 2610203C22R | RIKEN cDNA 2610203C22 gene | −5 | −6 | −4 | −4 |
| | ik | | | | | |
| 55 | Abcg3 | ATP binding cassette subfamily G member 3 | −5 | −7 | −13 | −9 |
| 56 | Star | steroidogenic acute regulatory protein | −5 | −1 | −914 | −588 |
| 57 | Grik2 | glutamate receptor, ionotropic, kainate 2 (beta 2) | −5 | −2 | −39 | −6 |
| 58 | Grin2c | glutamate receptor, ionotropic, NMDA2C (epsilon 3) | −5 | −2 | −5 | −3 |
| 59 | Vstm5 | V-set and transmembrane domain containing 5 | −5 | −4 | −3 | −2 |
| 60 | Smad9 | SMAD family member 9 | −5 | −6 | −4 | −6 |
| 61 | Pianp | PILR alpha associated neural protein | −5 | −4 | −12 | −5 |
| 62 | Rnf144a | ring finger protein 144A | −5 | −5 | −2 | −2 |
| 63 | Cthrc1 | collagen triple helix repeat containing 1 | −5 | −6 | −16 | −3 |
| 64 | Nxpe4 | neurexophilin and PC-esterase domain family, member 4 | −5 | −4 | −2 | −2 |
| 65 | Mrc2 | mannose receptor, C type 2 | −5 | −3 | −2 | −2 |
| 66 | Cx3cr1 | chemokine (C-X3-C motif) receptor 1 | −5 | −9 | −5 | −13 |
| 67 | Il5ra | interleukin 5 receptor, alpha | −5 | −3 | −4 | −8 |
| 68 | Kcnc4 | potassium voltage gated channel, Shaw-related subfamily, | −5 | −2 | −5 | −3 |
| | | member 4 | | | | |
| 69 | Fam163a | family with sequence similarity 163, member A | −5 | −18 | −2 | −2 |
| 70 | Cxcr3 | chemokine (C-X-C motif) receptor 3 | −5 | −3 | −3 | −4 |
| 71 | A330074K22 | RIKEN cDNA A330074K22 gene | −5 | −12 | −75 | −25 |
| | Rik | | | | | |
| 72 | Gm11627 | predicted gene 11627 | −5 | −3 | −13 | −7 |
| 73 | Mmp11 | matrix metallopeptidase 11 | −5 | −6 | −2 | −1 |
| 74 | Fam181b | family with sequence similarity 181, member B | −5 | −5 | −3 | −5 |
| 75 | Cd209b | CD209b antigen | −5 | −2 | −53 | −5 |
| 76 | Tnfrsf19 | tumor necrosis factor receptor superfamily, member 19 | −5 | −4 | −27 | −19 |
| 77 | Sept4 | septin 4 | −4 | −3 | −4 | −3 |
| 78 | Nlgn3 | neuroligin 3 | −4 | −3 | −173 | −426 |
| 79 | Baalc | brain and acute leukemia, cytoplasmic | −4 | −2 | −9 | −5 |
| 80 | Ngb | neuroglobin | −4 | −4 | −10 | −4 |
| 81 | Nxph3 | neurexophilin 3 | −4 | −4 | −9 | −2 |
| 82 | Klb | klotho beta | −4 | −2 | −5 | −3 |
| 83 | Igkv4-72 | immunoglobulin kappa chain variable 4-72 | −4 | −7 | −31 | −20 |
| 84 | Ptprv | protein tyrosine phosphatase receptor type V | −4 | −3 | −30 | −2 |
| 85 | Nrep | neuronal regeneration related protein | −4 | −2 | −3 | −9 |
| 86 | Itga8 | integrin alpha 8 | −4 | −2 | −3 | −1 |
| 87 | Myct1 | myc target 1 | −4 | −8 | −5 | −10 |
| 88 | Palm | paralemmin | −4 | −4 | −2 | −2 |
| 89 | Syt3 | synaptotagmin III | −4 | −2 | −2 | −1 |
| 90 | Tspan18 | tetraspanin 18 | −4 | −2 | −2 | −3 |
| 91 | Piezo2 | piezo-type mechanosensitive ion channel component 2 | −4 | −3 | −3 | −1 |
| 92 | Nat8f3 | N-acetyltransferase 8 (GCN5-related) family member 3 | −4 | −9 | −2 | −3 |
| 93 | Cngb1 | cyclic nucleotide gated channel beta 1 | −4 | −1 | −9 | −2 |
| 94 | Cd209g | CD209g antigen | −4 | −19 | −32 | −4 |
| 95 | Faim2 | Fas apoptotic inhibitory molecule 2 | −4 | −3 | −6 | −13 |
| 96 | Gfra2 | glial cell line derived neurotrophic factor family receptor | −4 | −3 | −5 | −4 |
| | | alpha 2 | | | | |
| 97 | Srpk3 | serine/arginine-rich protein specific kinase 3 | −4 | −3 | −74 | −3 |
| 98 | Gm3716 | predicted gene 3716 | −4 | −4 | −7 | −6 |
| 99 | Klhl13 | kelch-like 13 | −4 | −3 | −3 | −2 |
| 100 | Mogat2 | monoacylglycerol O-acyltransferase 2 | −4 | −6 | −2 | −1 |

The NTCI also increased the expression of the gene encoding oxytocin receptor (Oxtr), a 7-transmembrane G protein-coupled receptor that activates a set of signaling cascades, such as the MAPK, PKC, PLC, or CaMK pathways, which converge on transcription factors CREB or MEF-2. OXTR regulates neurite outgrowth, cellular viability, and increased survival. NTCI partially restored the Oxtr gene expression in the lungs and kidneys (Table 2). Likewise, NTCI increased another sepsis-downregulated gene encoding apelin receptor (Aplnr) in the lungs but decreased its expression in the kidneys (Table 2). Significantly, in cellular signaling, apelin is an endogenous peptide ligand for the APJ receptor involved in angiogenesis, cellular homeostasis, cardiovascular maintenance, and neuroprotection. In response to apelin, the APJ receptor drives the PI3K/Akt, MAPK, and PKA signaling pathways, leading to cell proliferation and protection from excitotoxicity. Similarly, the gene encoding β-Klotho was downregulated by sepsis in the lungs and kidneys and increased by the NTCI. This essential component of fibroblast growth factor (FGF) receptor complexes is required for high-affinity binding of endocrine FGF19 and FGF21 to evoke the signaling cascade actively involved in homeostatic maintenance of glucose metabolism and energy expenditure that are dysregulated in sepsis.

(5) the Top 100 Most Responsive Lung Genes in Sepsis and their Regulation by the Selective Nuclear Blockade with the NTCI

Next, we focused on the top 100 most upregulated and 100 most downregulated genes by sepsis in the lungs and determined the impact of the selective nuclear blockade with NTCI on their expression (Tables 3 and 4).

TABLE 3
Genes with Increased Expression in Lungs of Infected Mice

| | | | Lungs | |
|---|---|---|---|---|
| | Gene ID | Gene Name | Saline | NTCI |
| 1 | Saa2 | serum amyloid A 2 | 3,985 | 1,338 |
| 2 | Saa1 | serum amyloid A 1 | 1,849 | 658 |
| 3 | Prok2 | prokineticin 2 | 1,783 | 450 |
| 4 | B230303A05Rik | U1 small nuclear ribonucleoprotein 1C pseudogene | 1,612 | 697 |
| 5 | Gm43079 | predicted gene 43079 | 423 | 1 |
| 6 | Gm49383 | predicted gene, 49383 | 309 | 167 |
| 7 | Cldn14 | claudin 14 | 270 | 119 |
| 8 | Gm15056 | predicted gene 15056 | 200 | 17 |
| 9 | U90926 | cDNA sequence U90926 | 190 | 10 |
| 10 | Stfa2 | stefin A2 | 185 | 90 |
| 11 | Prokr2 | prokineticin receptor 2 | 171 | 5 |
| 12 | Lbx1 | ladybird homeobox 1 | 167 | 105 |
| 13 | Cstdc5 | cystatin domain containing 5 | 123 | 101 |
| 14 | Tarm1 | T cell-interacting, activating receptor on myeloid cells 1 | 119 | 41 |
| 15 | Mrgpra2a | MAS-related GPR, member A2A | 88 | 64 |
| 16 | Cstdc4 | cystatin domain containing 4 | 88 | 80 |
| 17 | Gm48786 | predicted gene, 48786 | 84 | 32 |
| 18 | Gm19510 | predicted gene, 19510 | 84 | 4 |
| 19 | Gm32089 | predicted gene, 32089 | 74 | 29 |
| 20 | Gm5966 | predicted gene 5966 | 67 | 30 |
| 21 | Stfa3 | stefin A3 | 63 | 39 |
| 22 | Tmem132e | transmembrane protein 132E | 58 | 87 |
| 23 | Orm2 | orosomucoid 2 | 56 | 12 |
| 24 | Gm28551 | predicted gene 28551 | 56 | 63 |
| 25 | A630023A22Rik | RIKEN cDNA A630023A22 gene | 55 | 50 |
| 26 | Gm7457 | predicted gene 7457 | 54 | 43 |
| 27 | Serpina7 | serine (or cysteine) peptidase inhibitor, clade A (alpha-1 antiproteinase, | 53 | 25 |
| | | antitrypsin), member 7 | | |
| 28 | Bmerb1 | bMERB domain containing 1 | 49 | 28 |
| 29 | Klk9 | kallikrein related-peptidase 9 | 48 | 29 |
| 30 | 1700011B04Rik | RIKEN cDNA 1700011B04 gene | 48 | 15 |
| 31 | Asprv1 | aspartic peptidase, retroviral-like 1 | 48 | 12 |
| 32 | Dmbt1 | deleted in malignant brain tumors 1 | 48 | 118 |
| 33 | Tmprss11d | transmembrane protease, serine 11d | 47 | 7 |
| 34 | Slc5a7 | solute carrier family 5 (choline transporter), member 7 | 43 | 18 |
| 35 | Ifitm7 | interferon induced transmembrane protein 7 | 42 | 17 |
| 36 | Gm38340 | predicted gene, 38340 | 41 | 26 |
| 37 | Pglyrp3 | peptidoglycan recognition protein 3 | 41 | 22 |
| 38 | Gm26748 | predicted gene, 26748 | 40 | 13 |
| 39 | 4930431P03Rik | RIKEN cDNA 4930431P03 gene | 39 | 11 |
| 40 | 4732490B19Rik | RIKEN cDNA 4732490B19 gene | 38 | 134 |
| 41 | Sectm1b | secreted and transmembrane 1B | 37 | 38 |
| 42 | Gm4948 | trans-2,3-enoyl-CoA reductase pseudogene | 36 | 229 |
| 43 | Gm20406 | predicted gene, 20406 | 33 | 5 |
| 44 | Rasl10b | RAS-like, family 10, member B | 33 | 13 |
| 45 | Gm36079 | annexin A2 receptor 2 | 32 | 12 |
| 46 | Sult2a3 | sulfotransferase family 2A, dehydroepiandrosterone (DHEA)-preferring, | 32 | 4 |
| | | member 3 | | |
| 47 | Apcs | amyloid P component, serum | 32 | 11 |
| 48 | Mymk | myomaker, myoblast fusion factor | 31 | 11 |
| 49 | Nkx2-9 | NK2 homeobox 9 | 29 | 4 |
| 50 | Gm26535 | predicted gene, 26535 | 29 | 7 |
| 51 | Rnase2a | ribonuclease, RNase A family, 2A (liver, eosinophil-derived neurotoxin) | 29 | 6 |
| 52 | Slc22a20 | solute carrier family 22 (organic anion transporter), member 20 | 28 | 6 |
| 53 | Stfa211 | stefin A2 like 1 | 28 | 24 |
| 54 | Gm4949 | glutamate-ammonia ligase (glutamine synthetase) pseudogene | 28 | 81 |
| 55 | Gm11738 | predicted gene 11738 | 27 | 556 |
| 56 | Gm29686 | predicted gene, 29686 | 27 | 9 |
| 57 | Serpine3 | serpin peptidase inhibitor, clade E (nexin, plasminogen activator inhibitor | 27 | 10 |
| | | type 1), member 3 | | |
| 58 | Gm807 | predicted gene 807 | 26 | 45 |
| 59 | Gm26444 | predicted gene, 26444 | 26 | 16 |
| 60 | Trdv5 | T cell receptor delta variable 5 | 25 | 4 |
| 61 | Sult1e1 | sulfotransferase family 1E, member 1 | 25 | 9 |
| 62 | Serpina3e-ps | serine (or cysteine) peptidase inhibitor, clade A, member 3E, pseudogene | 25 | 16 |
| 63 | 4930578G10Rik | RIKEN cDNA 4930578G10 gene | 24 | 22 |
| 64 | Gm37645 | predicted gene, 37645 | 24 | 4 |
| 65 | Ceacam19 | CEA cell adhesion molecule 19 | 23 | 3 |
| 66 | Ackr1 | atypical chemokine receptor 1 (Duffy blood group) | 23 | 12 |
| 67 | Ms4a3 | membrane-spanning 4-domains, subfamily A, member 3 | 23 | 2 |
| 68 | B130046B21Rik | RIKEN cDNA B130046B21 gene | 23 | 30 |
| 69 | Ctcflos | CCCTC-binding factor (zinc finger protein)-like, opposite strand | 23 | 17 |
| 70 | Doc2b | double C2, beta | 23 | 24 |
| 71 | Gm49573 | predicted gene, 49573 | 22 | 1 |
| 72 | Gm38275 | predicted gene, 38275 | 21 | 6 |
| 73 | Tex44 | testis expressed 44 | 21 | 2 |
| 74 | Gpr22 | G protein-coupled receptor 22 | 21 | 11 |
| 75 | Ceacam10 | CEA cell adhesion molecule 10 | 21 | 16 |
| 76 | E030018B13Rik | RIKEN cDNA E030018B13 gene | 20 | 6 |
| 77 | Gm37131 | predicted gene, 37131 | 20 | 14 |
| 78 | Htr3a | 5-hydroxytryptamine (serotonin) receptor 3A | 20 | 9 |
| 79 | Slc13a5 | solute carrier family 13 (sodium-dependent citrate transporter), member 5 | 20 | 7 |
| 80 | Nxf3 | nuclear RNA export factor 3 | 19 | 7 |
| 81 | Gm49427 | predicted gene, 49427 | 19 | 3 |
| 82 | Pde6h | phosphodiesterase 6H, cGMP-specific, cone, gamma | 19 | 10 |
| 83 | Ubd | ubiquitin D | 19 | 5 |
| 84 | Fcrlb | Fc receptor-like B | 19 | 8 |
| 85 | Sult2a5 | sulfotransferase family 2A, dehydroepiandrosterone (DHEA)-preferring, | 19 | 4 |
| | | member 5 | | |
| 86 | Tat | tyrosine aminotransferase | 18 | 11 |
| 87 | Gm6610 | predicted gene 6610 | 18 | −2 |
| 88 | Gm44292 | predicted gene, 44292 | 18 | 21 |
| 89 | Gm44165 | predicted gene, 44165 | 17 | 12 |
| 90 | Prss22 | protease, serine 22 | 17 | 41 |
| 91 | Gm11714 | predicted gene 11714 | 16 | 32 |
| 92 | Gm49368 | predicted gene, 49368 | 16 | 3 |
| 93 | Gm48065 | predicted gene, 48065 | 16 | 23 |
| 94 | Aoah | acyloxyacyl hydrolase | 16 | 6 |
| 95 | Tctex1d1 | dynein light chain Tctex-type 5 | 16 | 3 |
| 96 | Phox2a | paired-like homeobox 2a | 16 | 19 |
| 97 | Slc36a3 | solute carrier family 36 (proton/amino acid symporter), member 3 | 15 | 6 |
| 98 | Sult2a1 | sulfotransferase family 2A, dehydroepiandrosterone (DHEA)-preferring, | 15 | 3 |
| | | member 1 | | |
| 99 | Gm37347 | predicted gene, 37347 | 15 | 7 |
| 100 | Gm50083 | predicted gene, 50083 | 15 | 4 |

TABLE 4
Genes with Decreased Expression in Lungs of Infected Mice

| | | | Lungs | |
|---|---|---|---|---|
| | Gene ID | Gene Name | Saline | NTCI |
| 1 | Tmem233 | transmembrane protein 233 | −77 | NS |
| 2 | Duxbl3 | double homeobox B-like 1 | −71 | −2 |
| 3 | Rnase1 | ribonuclease, RNase A family, 1 (pancreatic) | −58 | NS |
| 4 | Defb42 | defensin beta 42 | −57 | NS |
| 5 | AL935121.1 | predicted gene, 52968 | −54 | −2 |
| 6 | Zp1 | zona pellucida glycoprotein 1 | −53 | −14 |
| 7 | Snurf | SNRPN upstream reading frame | −47 | NS |
| 8 | Gm25541 | predicted gene, 25541 | −45 | −19 |
| 9 | 4933403008Rik | RIKEN cDNA 4933403008 gene aka. testis expressed gene 16 (Tex16) | −40 | NS |
| 10 | 1810046K07Rik | POU domain, class 2, associating factor 2 | −40 | −7 |
| 11 | Gm5639 | PWWP domain containing 4, pseudogene | −38 | −3 |
| 12 | Myh2 | myosin, heavy polypeptide 2, skeletal muscle, adult | −35 | −5 |
| 13 | Gm765 | MyoD family inhibitor domain containing 2 | −35 | −6 |
| 14 | Gm50242 | predicted gene, 50242 | −34 | −9 |
| 15 | Gm6018 | MAD1 mitotic arrest deficient 1-like 1 pseudogene | −33 | NS |
| 16 | Ackr4 | atypical chemokine receptor 4 | −28 | −17 |
| 17 | Tnfrsf17 | tumor necrosis factor receptor superfamily, member 17 | −28 | −1 |
| 18 | Gm37858 | predicted gene, 37858 | −28 | −16 |
| 19 | Igkv14-130 | immunoglobulin kappa variable 14-130 | −27 | −364 |
| 20 | Ptprh | protein tyrosine phosphatase receptor type H | −26 | −14 |
| 21 | Gpr165 | G protein-coupled receptor 165 | −25 | NS |
| 22 | Gm47652 | predicted gene, 47652 | −25 | −5 |
| 23 | Gm16534 | family with sequence similarity 78, member A | −23 | −1 |
| 24 | 0610012D04Rik | RIKEN cDNA 0610012D04 gene | −23 | −13 |
| 25 | Gm36569 | predicted gene, 36569 | −23 | −5 |
| 26 | Gzmk | granzyme K | −23 | −8 |
| 27 | Gm9918 | predicted gene 9918 | −21 | −2 |
| 28 | Gm33677 | predicted gene, 33677 | −19 | −1 |
| 29 | St6gal2 | beta galactoside alpha 2,6 sialyltransferase 2 | −19 | −6 |
| 30 | Krtap17-1 | keratin associated protein 17-1 | −18 | −3 |
| 31 | Olfr1342 | olfactory receptor family 13 subfamily P member 4 | −17 | −136 |
| 32 | Lhcgr | luteinizing hormone/choriogonadotropin receptor | −17 | −6 |
| 33 | Ccdc42 | coiled-coil domain containing 42 | −16 | −9 |
| 34 | Zfp985 | zinc finger protein 985 | −16 | −4 |
| 35 | Igkv4-70 | immunoglobulin kappa chain variable 4-70 | −16 | −14 |
| 36 | Gm31517 | predicted gene, 31517 | −15 | −5 |
| 37 | Gm9767 | predicted gene 9767 | −14 | −1 |
| 38 | Olfr71 | olfactory receptor family 13 subfamily J member 1 | −14 | −13 |
| 39 | Slc26a3 | solute carrier family 26, member 3 | −13 | −10 |
| 40 | Trpv3 | transient receptor potential cation channel, subfamily V, member 3 | −13 | −4 |
| 41 | Gm49727 | predicted gene, 49727 | −13 | −8 |
| 42 | Gm43951 | RIKEN cDNA 4933427D14 gene | −13 | −2 |
| 43 | E330018M18Rik | RIKEN cDNA E330018M18 gene | −13 | −10 |
| 44 | Calcb | calcitonin-related polypeptide, beta | −13 | −3 |
| 45 | Rspo2 | R-spondin 2 | −12 | −18 |
| 46 | Glp1r | glucagon-like peptide 1 receptor | −12 | −13 |
| 47 | Fam124b | family with sequence similarity 124, member B | −12 | −10 |
| 48 | Amtn | amelotin | −12 | −4 |
| 49 | 4930570G19Rik | RIKEN cDNA 4930570G19 gene | −12 | −3 |
| 50 | Gm11574 | predicted gene 11574 | −12 | −5 |
| 51 | Pla2g2e | phospholipase A2, group IIE | −11 | −1 |
| 52 | Sfrp2 | secreted frizzled-related protein 2 | −11 | −7 |
| 53 | Pigz | phosphatidylinositol glycan anchor biosynthesis, class Z | −11 | −7 |
| 54 | Gm13688 | predicted gene 13688 | −11 | −3 |
| 55 | Abca14 | ATP-binding cassette, sub-family A (ABC1), member 14 | −11 | −1 |
| 56 | B530045E10Rik | RIKEN cDNA B530045E10 gene | −10 | −4 |
| 57 | Fras1 | Fraser extracellular matrix complex subunit 1 | −10 | −3 |
| 58 | Gm12159 | predicted gene 12159 | −10 | −2 |
| 59 | Gm48488 | predicted gene, 48488 | −10 | −2 |
| 60 | 9030404E10Rik | RIKEN cDNA 9030404E10 gene | −10 | −12 |
| 61 | Hmcn1 | hemicentin 1 | −10 | −3 |
| 62 | Igkv4-68 | immunoglobulin kappa variable 4-68 | −10 | −15 |
| 63 | Wnt7a | wingless-type MMTV integration site family, member 7A | −10 | −15 |
| 64 | Tmem74 | transmembrane protein 74 | −10 | −6 |
| 65 | Ighv1-66 | immunoglobulin heavy variable 1-66 | −10 | −31 |
| 66 | Cr2 | complement receptor 2 | −9 | −6 |
| 67 | Slc5a11 | solute carrier family 5 (sodium/glucose cotransporter), member 11 | −9 | −71 |
| 68 | Lrrc17 | leucine rich repeat containing 17 | −9 | −7 |
| 69 | Slc22a14 | solute carrier family 22 (organic cation transporter), member 14 | −9 | −2 |
| 70 | Dipk2b | divergent protein kinase domain 2B | −9 | −11 |
| 71 | Fgf18 | fibroblast growth factor 18 | −9 | −6 |
| 72 | Ndp | Norrie disease (pseudoglioma) (human) | −9 | −5 |
| 73 | Gm17334 | predicted gene, 17334 | −9 | −6 |
| 74 | Btnl2 | butyrophilin-like 2 | −9 | −7 |
| 75 | Gm10432 | predicted gene 10432 | −9 | −4 |
| 76 | Colq | collagen-like tail subunit (single strand of homotrimer) of asymmetric | −9 | −3 |
| | | acetylcholinesterase | | |
| 77 | Gm48662 | predicted gene, 48662 | −9 | −7 |
| 78 | 4833412C05Rik | RIKEN cDNA 4833412C05 gene | −9 | −3 |
| 79 | Gm12576 | predicted gene 12576 | −9 | −5 |
| 80 | Igkv6-17 | immunoglobulin kappa variable 6-17 | −9 | −8 |
| 81 | Gm13816 | predicted gene 13816 | −8 | −8 |
| 82 | Tmem266 | transmembrane protein 266 | −8 | 1 |
| 83 | Ighv5-6 | immunoglobulin heavy variable 5-6 | −8 | −67 |
| 84 | Cntn4 | contactin 4 | −8 | −2 |
| 85 | Rbp7 | retinol binding protein 7, cellular | −8 | −2 |
| 86 | Insyn2a | inhibitory synaptic factor 2A | −8 | −3 |
| 87 | Slc5a12 | solute carrier family 5 (sodium/glucose cotransporter), member 12 | −8 | −3 |
| 88 | Gm9947 | predicted gene 9947 | −8 | −3 |
| 89 | Igkv12-89 | immunoglobulin kappa chain variable 12-89 | −8 | −7 |
| 90 | Gm26795 | predicted gene, 26795 | −8 | −2 |
| 91 | Ddx4 | DEAD box helicase 4 | −8 | −2 |
| 92 | Dclk3 | doublecortin-like kinase 3 | −8 | −16 |
| 93 | Hpcal4 | hippocalcin-like 4 | −8 | −3 |
| 94 | Ndnf | neuron-derived neurotrophic factor | −7 | −8 |
| 95 | Igkv17-121 | immunoglobulin kappa variable 17-121 | −7 | −10 |
| 96 | Slurp1 | secreted Ly6/Plaur domain containing 1 | −7 | −3 |
| 97 | Ano4 | anoctamin 4 | −7 | −2 |
| 98 | Ighv1-7 | immunoglobulin heavy variable V1-7 | −7 | −3 |
| 99 | Rimklb | ribosomal modification protein rimK-like family member B | −7 | −5 |
| 100 | Igkv4-80 | immunoglobulin kappa variable 4-80 | −7 | −10 |

Among the most sepsis-upregulated genes in the lungs, we noted the top 14 genes displaying remarkable rise of 119-fold to 3,985-fold in their upward expression shown in Table 3. Three of the most upregulated genes encode Saa2, Saa1, and Prok2. Whereas serum amyloid genes (Saa1, Saa2, and Saa3) were addressed earlier, Prok2 (upregulated 1,783-fold), that encodes prokineticin 2 is also noteworthy. Its deficiency is linked to the low levels of gonadotropins and testosterone with or without anosmia. The NTCI treatment reduced its expression to 450-fold level while the gene encoding Prok2 receptor (Prokr2) was reduced to 4.5-fold level by the NTCI. The role of prokineticin 2 and its receptor in the development of ARDS and other lesions of acute lung injury in sepsis is unknown although anosmia is a prominent feature of SARS Coronavirus-2 infections.

Two other genes strongly responding to sepsis are of particular interest: Tarm1 and Mrgprb2. The former encodes T cell-interacting activating receptor on myeloid cells. This novel leukocyte receptor expressed in neutrophils and macrophages plays an important role in proinflammatory response to acute bacterial infections. Simultaneous stimulation of TARM-1 and Toll-like receptor 4 enhanced the production of TNFα and IL-6 by granulocytes and macrophages. In turn, Mrgpra2a is the homolog to the human gene encoding MRGPRX2. This important protein mediates mast-cell activation in response to a broad array of stimuli ranging from wasp venom to several pharmaceutical compounds associated with the IgE-independent pseudo-allergic reactions in patients. The NTCI treatment reduced its expression in the lungs (Table 3).

Among the most downregulated lung genes in sepsis, is defensin beta 42 (Defb42) displaying predominantly anti-bacterial activity against Escherichia coli and Staphylococcus aureus. The expression of this gene in the lungs of NTCI-treated mice was not significant (Table 4).

(6) the Top 100 Most Responsive Kidney Genes in Sepsis and their Regulation by the Selective Nuclear Blockade with the NTCI

Among the top 100 most upregulated genes by sepsis in the kidneys (Tables 5 and 6), is eosinophil peroxidase (Epx) which displayed 118-fold increase. It was almost doubled by the NTCI to 231-fold level. As no clinical findings have been associated with human Epx deficiency, its overexpression in the kidneys during sepsis awaits further studies. In turn, G protein-coupled bile acid receptor-1 (Gpbar1 aka Takeda G protein-coupled receptor-5 [TGR5]) regulates bile acid metabolism and glucose and insulin sensitivity. TGR5 stimulates glucagon-like peptide-1 (GLP-1) secretion to improve insulin sensitivity and hepatic metabolism. Significantly, the treatment with the NTCI increased 4-fold the expression of the TGR5 underscoring the ability to have metabolic improvement in sepsis.

Among the genes in the kidneys displaying extreme downregulation during sepsis, the two genes encoding cytochrome P450, family 11 subfamily b and a polypeptide 1 were restored by the NTCI to higher albeit still abnormal levels. Along with the genes encoding cytochrome P450 family 17 subfamily A member 1, they participate in steroidogenesis being induced by endothelin-1. The profound suppression of these genes indicates potential failure of physiologic steroidogenesis in sepsis.

TABLE 5
Genes with Increased Expression in Kidneys of Infected Mice

| | | | Kidneys | |
|---|---|---|---|---|
| | Gene ID | Gene Name | Saline | NTCI |
| 1 | Epx | eosinophil peroxidase | 118 | 231 |
| 2 | Gm26965 | predicted gene, 26965 | 100 | 30 |
| 3 | Reg3g | regenerating islet-derived 3 gamma | 83 | 116 |
| 4 | Cela3b | chymotrypsin-like elastase family, member 3B | 70 | −1 |
| 5 | Gm12503 | predicted gene 12503 | 64 | 32 |
| 6 | Gpbar1 | G protein-coupled bile acid receptor 1 | 60 | 266 |
| 7 | Dnah6 | dynein, axonemal, heavy chain 6 | 58 | 47 |
| 8 | Fut2 | fucosyltransferase 2 | 50 | 252 |
| 9 | Muc4 | mucin 4 | 49 | 87 |
| 10 | Gm43189 | predicted gene 43189 | 37 | 15 |
| 11 | Angptl7 | angiopoietin-like 7 | 37 | 11 |
| 12 | Pcdh8 | protocadherin 8 | 32 | 49 |
| 13 | Sftpa1 | surfactant associated protein A1 | 32 | 21 |
| 14 | Mlana | melan-A | 32 | 10 |
| 15 | Gm16350 | predicted gene 16350 | 31 | 24 |
| 16 | Syt2 | synaptotagmin II | 30 | 15 |
| 17 | Gm17055 | predicted gene 17055 | 29 | 29 |
| 18 | Ppy | pancreatic polypeptide | 29 | 4 |
| 19 | Ccdc33 | coiled-coil domain containing 33 | 29 | 10 |
| 20 | Gm33280 | predicted gene, 33280 | 28 | 9 |
| 21 | Gm45643 | predicted gene 45643 | 27 | 15 |
| 22 | Lrit3 | leucine-rich repeat, immunoglobulin-like and transmembrane domains 3 | 26 | 24 |
| 23 | Sbk2 | SH3-binding domain kinase family, member 2 | 26 | 26 |
| 24 | Slc6a19os | solute carrier family 6 (neurotransmitter transporter), member 19, opposite | 26 | 43 |
| | | strand | | |
| 25 | Slamf1 | signaling lymphocytic activation molecule family member 1 | 26 | 39 |
| 26 | Gm5541 | TD and POZ domain containing 4 pseudogene | 26 | 4 |
| 27 | Fgf21 | fibroblast growth factor 21 | 25 | 6 |
| 28 | Gm49392 | predicted gene, 49392 | 25 | 13 |
| 29 | Slc6a16 | solute carrier family 6, member 16 | 24 | −1 |
| 30 | Olr1 | oxidized low density lipoprotein (lectin-like) receptor 1 | 24 | 13 |
| 31 | Gm35028 | predicted gene, 35028 | 23 | 15 |
| 32 | Slfn4 | schlafen 4 | 23 | 14 |
| 33 | Gm20548 | predicted gene 20548 | 22 | 9 |
| 34 | Gm40709 | predicted gene, 40709 | 22 | 9 |
| 35 | Speer4f2 | spermatogenesis associated glutamate (E)-rich protein 4f2 | 21 | 19 |
| 36 | Gm13363 | predicted gene 13363 | 21 | 14 |
| 37 | Egr2 | early growth response 2 | 20 | 11 |
| 38 | Gm44260 | predicted gene, 44260 | 19 | 4 |
| 39 | Marc1 | mitochondrial amidoxime reducing component 1 | 19 | 23 |
| 40 | Crygn | crystallin, gamma N | 19 | 5 |
| 41 | Lpo | lactoperoxidase | 18 | 24 |
| 42 | Gabrp | gamma-aminobutyric acid (GABA) A receptor, pi | 17 | 15 |
| 43 | Bpifc | BPI fold containing family C | 17 | 5 |
| 44 | Fgf18 | fibroblast growth factor 18 | 17 | 11 |
| 45 | Lncenc1 | long non-coding RNA, embryonic stem cells expressed 1 | 17 | 9 |
| 46 | Prss27 | protease, serine 27 | 17 | 39 |
| 47 | Muc19 | mucin 19 | 16 | 7 |
| 48 | Gm39929 | predicted gene, 39929 | 16 | 4 |
| 49 | Gm2670 | predicted gene 2670 | 16 | 7 |
| 50 | 5830487J09Rik | RIKEN cDNA 5830487J09 gene | 15 | 10 |
| 51 | Olfr1442 | olfactory receptor family 5 subfamily B member 94 | 15 | 21 |
| 52 | Krt20 | keratin 20 | 14 | 30 |
| 53 | Gm19684 | predicted gene, 19684 | 14 | 2 |
| 54 | Gm33424 | predicted gene, 33424 | 14 | 55 |
| 55 | 4732419C18Rik | RIKEN cDNA 4732419C18 gene | 14 | 30 |
| 56 | Gm37621 | predicted gene, 37621 | 14 | 13 |
| 57 | Creb313 | CAMP responsive element binding protein 3-like 3 | 13 | 6 |
| 58 | Gm14319 | predicted gene 14319 | 13 | 18 |
| 59 | Dnah10 | dynein, axonemal, heavy chain 10 | 12 | 12 |
| 60 | Areg | amphiregulin | 12 | 24 |
| 61 | Armc12 | armadillo repeat containing 12 | 12 | 29 |
| 62 | Eif4ebp3 | eukaryotic translation initiation factor 4E binding protein 3 | 12 | 20 |
| 63 | Gdf6 | growth differentiation factor 6 | 11 | 5 |
| 64 | Gm49037 | predicted gene, 49037 | 11 | 11 |
| 65 | Gm42793 | predicted gene 42793 | 11 | 41 |
| 66 | Derl3 | Der1-like domain family, member 3 | 11 | 5 |
| 67 | Nptx2 | neuronal pentraxin 2 | 11 | 23 |
| 68 | Atp12a | ATPase, H+/K+ transporting, nongastric, alpha polypeptide | 10 | 7 |
| 69 | Kcna7 | potassium voltage-gated channel, shaker-related subfamily, member 7 | 10 | 3 |
| 70 | Prg4 | proteoglycan 4 (megakaryocyte stimulating factor, articular superficial zone | 10 | 5 |
| | | protein) | | |
| 71 | Ctla4 | cytotoxic T-lymphocyte-associated protein 4 | 10 | 16 |
| 72 | Gm44164 | predicted gene, 44164 | 9 | 6 |
| 73 | Mmp7 | matrix metallopeptidase 7 | 9 | 5 |
| 74 | Gm44949 | predicted gene 44949 | 9 | 9 |
| 75 | Slc17a8 | solute carrier family 17 (sodium-dependent inorganic phosphate | 9 | 3 |
| | | cotransporter), member 8 | | |
| 76 | Lamb3 | laminin, beta 3 | 9 | 6 |
| 77 | Plekhs1 | pleckstrin homology domain containing, family S member 1 | 9 | 19 |
| 78 | Trim17 | tripartite motif-containing 17 | 9 | 3 |
| 79 | Dusp8 | dual specificity phosphatase 8 | 9 | 6 |
| 80 | 4930422C21Rik | RIKEN cDNA 4930422C21 gene | 9 | 4 |
| 81 | Gm10727 | predicted gene 10727 | 9 | 2 |
| 82 | Col27a1 | collagen, type XXVII, alpha 1 | 8 | 9 |
| 83 | Tacc2 | transforming, acidic coiled-coil containing protein 2 | 8 | 6 |
| 84 | Fat2 | FAT atypical cadherin 2 | 8 | 2 |
| 85 | Lif | leukemia inhibitory factor | 8 | 20 |
| 86 | Klk1b1 | kallikrein 1-related peptidase b1 | 8 | 5 |
| 87 | Tsc22d1 | TSC22 domain family, member 1 | 8 | 11 |
| 88 | Wnt8b | wingless-type MMTV integration site family, member 8B | 8 | 20 |
| 89 | Grem1 | gremlin 1, DAN family BMP antagonist | 8 | 14 |
| 90 | Hesx1 | homeobox gene expressed in ES cells | 8 | 15 |
| 91 | Hhipl1 | hedgehog interacting protein-like 1 | 8 | 5 |
| 92 | Ugt1a5 | UDP glucuronosyltransferase 1 family, polypeptide A5 | 8 | 1 |
| 93 | Klk1b4 | kallikrein 1-related pepidase b4 | 7 | 15 |
| 94 | Gm8947 | predicted gene 8947 | 7 | 6 |
| 95 | A530013C23Rik | RIKEN cDNA A530013C23 gene | 7 | 9 |
| 96 | Mylk4 | myosin light chain kinase family, member 4 | 7 | 4 |
| 97 | Cyp2a5 | cytochrome P450, family 2, subfamily a, polypeptide 5 | 7 | 9 |
| 98 | Muc1 | mucin 1, transmembrane | 7 | 5 |
| 99 | Gm44588 | predicted gene 44588 | 7 | 3 |
| 100 | Gm45053 | predicted gene 45053 | 7 | 10 |

TABLE 6
Genes with Decreased Expression in Kidneys of Infected Mice

| | | | Kidneys | |
|---|---|---|---|---|
| | Gene ID | Gene Name | Saline | NTCI |
| 1 | Cyp11b1 | cytochrome P450, family 11, subfamily b, polypeptide 1 | −70,748 | −1,178 |
| 2 | Cyp11a1 | cytochrome P450, family 11, subfamily a, polypeptide 1 | −27,744 | −483 |
| 3 | Akr1cl | aldo-keto reductase family 1, member C-like | −17,566 | −884 |
| 4 | Hsd3b1 | hydroxy-delta-5-steroid dehydrogenase, 3 beta- and steroid delta-isomerase | −11,966 | −476 |
| | | 1 | | |
| 5 | Scg2 | secretogranin II | −4,862 | −674 |
| 6 | Th | tyrosine hydroxylase | −4,501 | −344 |
| 7 | Cyp21a1 | cytochrome P450, family 21, subfamily a, polypeptide 1 | −3,966 | −495 |
| 8 | Chga | chromogranin A | −3,318 | −793 |
| 9 | Cyp21a2-ps | cytochrome P450, family 21, subfamily a, polypeptide 2 pseudogene | −1,979 | −443 |
| 10 | Pnmt | phenylethanolamine-N-methyltransferase | −1,705 | −322 |
| 11 | Chgb | chromogranin B | −1,447 | −508 |
| 12 | Cyp11b2 | cytochrome P450, family 11, subfamily b, polypeptide 2 | −1,274 | −114 |
| 13 | Caln1 | calneuron 1 | −1,121 | NS |
| 14 | Kcnk9 | potassium channel, subfamily K, member 9 | −1,062 | −238 |
| 15 | Dbh | dopamine beta hydroxylase | −912 | −641 |
| 16 | Gm28382 | predicted gene 28382 | −869 | −348 |
| 17 | Pla2g2f | phospholipase A2, group IIF | −851 | NS |
| 18 | Nr5a1 | nuclear receptor subfamily 5, group A, member 1 | −843 | −988 |
| 19 | Pcsk1n | proprotein convertase subtilisin/kexin type 1 inhibitor | −707 | −97 |
| 20 | Mgarp | mitochondria localized glutamic acid rich protein | −666 | −244 |
| 21 | 4930486L24Rik | RIKEN cDNA 4930486L24 gene | −630 | −23 |
| 22 | Cryba4 | crystallin, beta A4 | −549 | −43 |
| 23 | Atp2b3 | ATPase, Ca++ transporting, plasma membrane 3 | −541 | NS |
| 24 | Resp18 | regulated endocrine-specific protein 18 | −510 | −332 |
| 25 | E330017L17Rik | RIKEN cDNA E330017L17 gene | −473 | −154 |
| 26 | Pcsk1 | proprotein convertase subtilisin/kexin type 1 | −468 | −57 |
| 27 | Snap25 | synaptosomal-associated protein 25 | −448 | NS |
| 28 | Rims4 | regulating synaptic membrane exocytosis 4 | −352 | −271 |
| 29 | Slc24a2 | solute carrier family 24 (sodium/potassium/calcium exchanger), member 2 | −344 | NS |
| 30 | Phox2b | paired-like homeobox 2b | −316 | NS |
| 31 | Slc18a2 | solute carrier family 18 (vesicular monoamine), member 2 | −286 | −31 |
| 32 | Eef1a2 | eukaryotic translation elongation factor 1 alpha 2 | −271 | −181 |
| 33 | Chrna3 | cholinergic receptor, nicotinic, alpha polypeptide 3 | −260 | −169 |
| 34 | Hand2os1 | Hand2, opposite strand 1 | −240 | NS |
| 35 | Cntnap5a | contactin associated protein-like 5A | −240 | −38 |
| 36 | Rasgrf1 | RAS protein-specific guanine nucleotide-releasing factor 1 | −236 | −26 |
| 37 | Hand2 | heart and neural crest derivatives expressed 2 | −226 | −951 |
| 38 | Pnmal1 | PNMA family member 8A | −221 | −144 |
| 39 | Necab2 | N-terminal EF-hand calcium binding protein 2 | −184 | −44 |
| 40 | Tbx20 | T-box 20 | −182 | NS |
| 41 | Stmn3 | stathmin-like 3 | −163 | −22 |
| 42 | Gm17322 | predicted gene, 17322 | −154 | −17 |
| 43 | St8sia3 | ST8 alpha-N-acetyl-neuraminide alpha-2,8-sialyltransferase 3 | −148 | NS |
| 44 | Rab33a | RAB33A, member RAS oncogene family | −145 | −97 |
| 45 | Synpr | synaptoporin | −140 | −11 |
| 46 | Rit2 | Ras-like without CAAX 2 | −110 | −11 |
| 47 | Camkv | CaM kinase-like vesicle-associated | −107 | NS |
| 48 | Gdap1 | ganglioside-induced differentiation-associated-protein 1 | −105 | NS |
| 49 | Soga3 | SOGA family member 3 | −99 | NS |
| 50 | Mdga2 | MAM domain containing glycosylphosphatidylinositol anchor 2 | −99 | −10 |
| 51 | Mab21l2 | mab-21-like 2 | −96 | −122 |
| 52 | Nrsn1 | neurensin 1 | −93 | −61 |
| 53 | Cadps | Ca2+-dependent secretion activator | −91 | −37 |
| 54 | Gnb3 | guanine nucleotide binding protein (G protein), beta 3 | −83 | NS |
| 55 | 1520401A03Rik | glycine rich extracellular protein 1 | −82 | NS |
| 56 | Nrg3 | neuregulin 3 | −82 | NS |
| 57 | Spock1 | sparc/osteonectin, cwcv and kazal-like domains proteoglycan 1 | −81 | −9 |
| 58 | Cdk5r2 | cyclin dependent kinase 5, regulatory subunit 2 (p39) | −78 | NS |
| 59 | Ina | internexin neuronal intermediate filament protein, alpha | −76 | −26 |
| 60 | Act16b | actin-like 6B | −75 | NS |
| 61 | Npy2r | neuropeptide Y receptor Y2 | −74 | −21 |
| 62 | Gal | galanin and GMAP prepropeptide | −70 | −49 |
| 63 | Nrsn2 | neurensin 2 | −69 | NS |
| 64 | Gabrg2 | gamma-aminobutyric acid (GABA) A receptor, subunit gamma 2 | −69 | NS |
| 65 | Gpr158 | G protein-coupled receptor 158 | −68 | NS |
| 66 | Slc7a14 | solute carrier family 7 (cationic amino acid transporter, y+ system), member | −68 | −32 |
| | | 14 | | |
| 67 | Sphkap | SPHK1 interactor, AKAP domain containing | −67 | −51 |
| 68 | Tmem255a | transmembrane protein 255A | −67 | −61 |
| 69 | 8430419K02Rik | RIKEN cDNA 8430419K02 gene | −66 | −3 |
| 70 | Scg3 | secretogranin III | −65 | −276 |
| 71 | Gria1 | glutamate receptor, ionotropic, AMPA1 (alpha 1) | −65 | −11 |
| 72 | Txndc2 | thioredoxin domain containing 2 (spermatozoa) | −63 | −59 |
| 73 | Gm27477 | predicted gene, 27477 | −63 | −3 |
| 74 | Gria2 | glutamate receptor, ionotropic, AMPA2 (alpha 2) | −62 | −154 |
| 75 | Gjd2 | gap junction protein, delta 2 | −62 | NS |
| 76 | Lrrtm2 | leucine rich repeat transmembrane neuronal 2 | −62 | −2 |
| 77 | Hsd3b6 | hydroxy-delta-5-steroid dehydrogenase, 3 beta- and steroid delta-isomerase | −62 | −52 |
| | | 6 | | |
| 78 | Diras1 | DIRAS family, GTP-binding RAS-like 1 | −62 | −40 |
| 79 | Omg | oligodendrocyte myelin glycoprotein | −61 | NS |
| 80 | Gm49391 | predicted gene, 49391 | −60 | −6 |
| 81 | Fam155a | NALCN channel auxiliary factor 1 | −59 | −146 |
| 82 | Gm2007 | predicted gene 2007 | −59 | −5 |
| 83 | Kcnq2 | potassium voltage-gated channel, subfamily Q, member 2 | −58 | −38 |
| 84 | Gm42372 | PNMA family member 8C | −57 | −6 |
| 85 | Isl1 | ISL1 transcription factor, LIM/homeodomain | −57 | −7 |
| 86 | Lingo3 | leucine rich repeat and Ig domain containing 3 | −57 | −20 |
| 87 | Prl8a2 | prolactin family 8, subfamily a, member 2 | −57 | NS |
| 88 | Adh7 | alcohol dehydrogenase 7 (class IV), mu or sigma polypeptide | −56 | −20 |
| 89 | Lgi1 | leucine-rich repeat LGI family, member 1 | −53 | −7 |
| 90 | Gm47815 | predicted gene, 47815 | −53 | NS |
| 91 | Gm14592 | splicing factor 3a, subunit 3 pseudogene | −52 | NS |
| 92 | Cdx4 | caudal type homeobox 4 | −52 | −21 |
| 93 | Marcksl1-ps4 | MARCKS-like 1, pseudogene 4 | −52 | −3 |
| 94 | Sstr3 | somatostatin receptor 3 | −48 | −3 |
| 95 | Pcdh10 | protocadherin 10 | −47 | −9 |
| 96 | Gm27000 | predicted gene, 27000 | −47 | NS |
| 97 | Tub | tubby bipartite transcription factor | −47 | −20 |
| 98 | Mc2r | melanocortin 2 receptor | −46 | −53 |
| 99 | Il20ra | interleukin 20 receptor, alpha | −44 | −7 |
| 100 | Grin2a | glutamate receptor, ionotropic, NMDA2A (epsilon 1) | −43 | −28 |

### (7) Enhancement of Bacterial Clearance in Polymicrobial Sepsis by the Selective Nuclear Blockade.

As indicated above, sepsis had a significant impact on the genes encoding multiple mediators of innate immunity and inflammation in the lungs and kidneys, including Solute Carrier Family 11 (Proton-Coupled Divalent Metal Ion Transporters, Member 1 (Slc11a1), aka natural resistance associated macrophage protein 1 (NRAMP). This gene is linked to the control of intracellular pathogens, and along with the gene encoding cathelicidin antimicrobial peptide (Camp) and defensins, contributes to the innate immunity-mediated bacterial clearance in the lungs and kidneys as well as blood and spleen. This process depends on the phagocytic system.

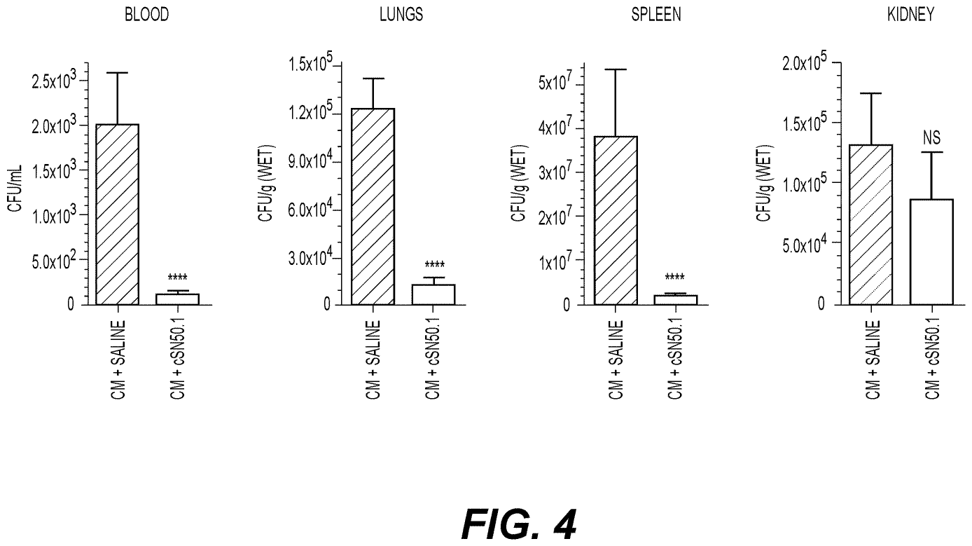

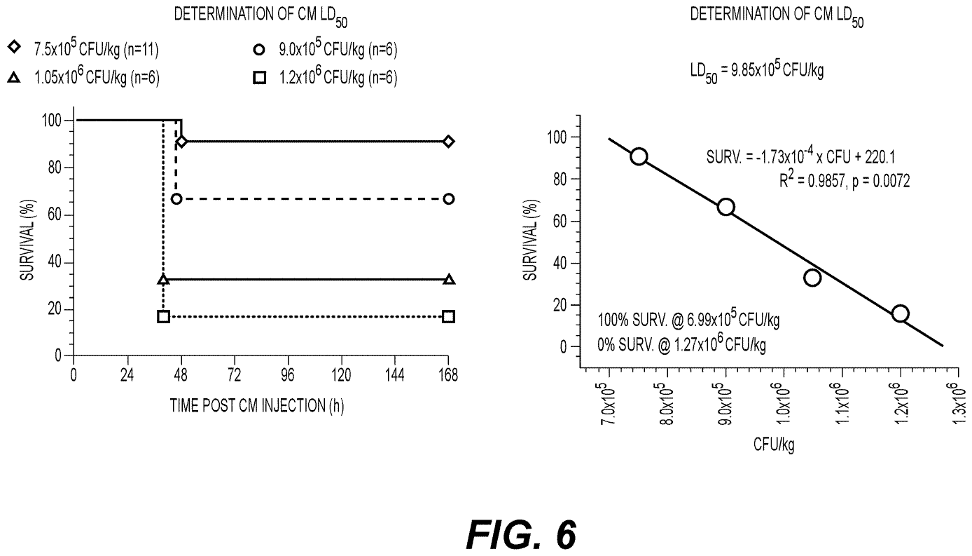

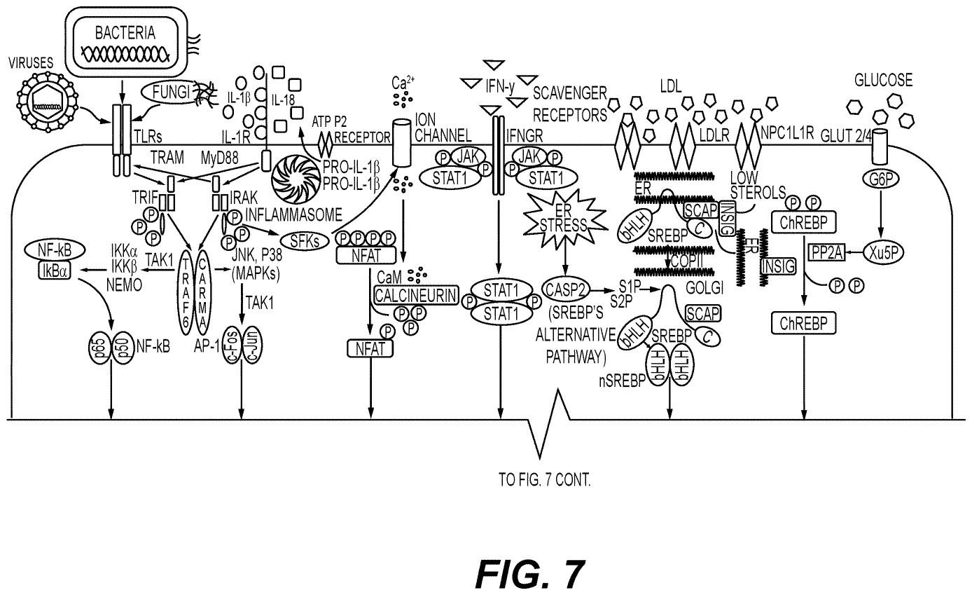

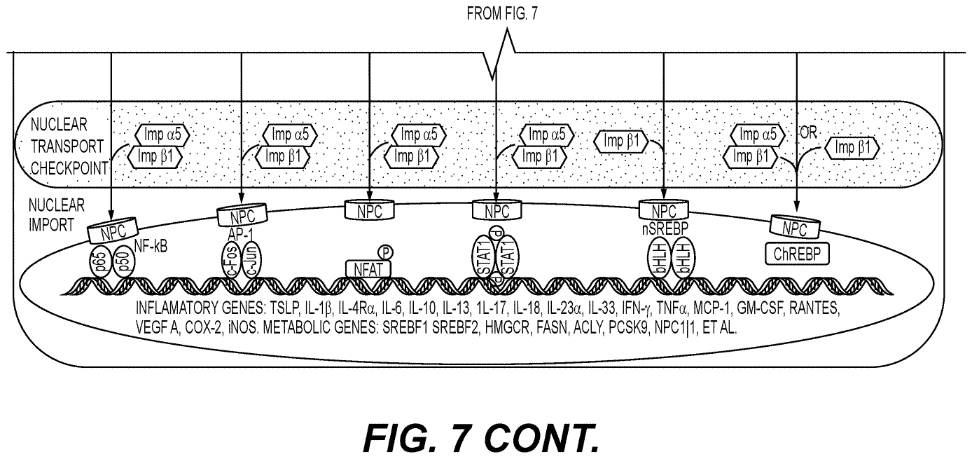

To study bacterial clearance in sepsis, we adopted its clinically relevant experimental model evolving from polymicrobial peritonitis. This non-surgical model provides consistent results in terms of the standardized microbial challenge as compared to the alternative surgical model, the cecal ligation and puncture. The latter is subject to the uncontrolled spillage of cecal microbiome into the peritoneal cavity. The cecal microbiome (abbreviated CM) was obtained from the euthanized healthy donor mice. The gut microbiome of healthy C57Bl/6 mice comprises: Dorea, Actinobacteria, Bifidobacteriaceae, Turucibacteriaceae, Firmicutes, Lactobacilliaceae, and other Bacilli. The mice were uniformly infected with a titrated amount of gut microbiota through a single intraperitoneal injection (4.5×10⁵ Colony Forming Units (CFU)/kg, representing half of the LD₅₀ determined for Cecal Microbiome (CM) (FIG. 6). We hasten to add that this microbial challenge comprises a multitude of intestinal Gram-negative bacteria expressing diverse LPS structures and additional virulence factors, Gram-positive bacteria, and other microbes residing in the gut microbiome. Importantly, the resulting polymicrobial peritonitis is accompanied by dissemination of the infection to the blood (bacteremia), lungs, spleen, kidneys, and other organs (FIG. 4), thereby producing a multi-system microbial inflammation that mediates sepsis.

Strikingly, the selective nuclear blockade with the parentally administered NTCI for 12 hours (without antimicrobial therapy) reduced the detectable bacterial count in the following ways: 18-fold in the blood, 9-fold in the spleen, 11-fold in the lungs (FIG. 4). The downward change of the bacterial count in the kidneys was not significant. We link these NTCI-induced gains in the bacterial clearance in the lungs, as compared to the kidneys, to the different genomic response to polymicrobial sepsis in these two target organs. We excluded the direct action of the NTCI on bacterial population comprising CM by using the Kirby-Bauer method of bacterial sensitivity testing with antibiotic meropenem as the positive control (Fig. SF4). We showed that the addition of NTCI to meropenem, a broad-spectrum β-lactam antibiotic, increased survival to 55% in a similar model of sepsis whereas meropenem alone afforded 30% survival.

### c) Discussion

We report that polymicrobial sepsis in the preclinical model causes a massive genomic response in the two main target organs, the lungs and kidneys. This unprecedented, major organ-based genomic response is attenuated by the selective nuclear blockade with the NTCI. Astoundingly, 3,735 sepsis-induced genes in the lungs and 1,951 sepsis-induced genes in the kidneys were reprogrammed by 6-hour treatment. Moreover, the NTCI-treated animals displayed the enhancement of microbial clearance that reduced the sepsis-causing dissemination of titrated gut microbiome following its intraperitoneal injection. The bacterial clearance was increased 18-fold in the blood, 9-fold in the spleen, and 11-fold in the lungs during the 12-hour treatment with the NTCI. Importantly, the 9-fold increase in microbial clearance in the spleen is clinically relevant. Patients with hyposplenism due to celiac disease or following bone marrow transplantation are at a significant risk of fulminant sepsis. Thus, the data sets presented herein and their impact on bacterial clearance in experimental sepsis serve as a translational platform for future clinical studies of patients with sepsis. Ultimately, the massive genomic response to sepsis, which is treated by the selective nuclear blockade, provides a foundation for much needed new therapeutic approaches. Our data also provide a basis for evaluation of the microbial clearance in other model systems to address the host's genomic response to the sepsis-causing viral, fungal, and protozoal pathogens. The prevention and very early control of severe viral, bacterial, fungal, and protozoal infections remains crucial for taming their progression into sepsis.

Other studies of the whole blood response to pediatric sepsis, reported over 1,000 upregulated and 1,401 downregulated genes that were expressed in blood cells based on genome-level expression profiling using the microarray technology. The whole blood response to adult sepsis has identified two classes of genomic changes: “adaptive” and “inflammatory”. The former was dominated by T cells and natural killer (NK) cells, which displayed an upregulation of the T cell receptor signaling pathway and NK cell-mediated cytotoxicity. The “inflammatory” response displayed a preponderance of macrophages with upregulation of the Toll-like receptor signaling pathway linked to a less favorable prognosis. Our study of the two major organs, the lungs and kidneys, identified over 6,200 sepsis-responding genes in the lungs, and over 5,400 genes in the kidneys that were changed downward or upward during the 6-hour treatment with the selective nuclear blockade.

While comparing the genomic response to sepsis in the lungs and kidneys, we can consider structural and functional differences between them. Distinct regions of the lungs display different population of epithelial cells that function as adult stem cells. These cells are differentiated. Furthermore, the mature epithelium differs along the proximal-distal axis thereby determining its different potential for repair in response to injury. In turn, the air-blood barrier comprising alveolar-microvascular unit is particularly vulnerable to sepsis-causing microbial agents. They include viruses, such as influenza and SARS-Coronavirus 1 and 2, and LPS-bearing Gram-negative bacteria, the cause of sepsis in two-thirds of patients, as well as Staphylococci and Streptococci producing superantigenic immunotoxins.

The kidneys differ from the lungs in terms of their cell types and gene expression. In contrast to the lungs, the kidney cells are surrounded by the gradient of increasing tissue osmolality from the cortex to the medulla thereby influencing their transcriptomes. The kidney's tubules traverse significant space from kidney's cortex to medulla. This space is marked by steep corticomedullary gradients of tissue osmolality and oxygen tension, known to influence the gene expression of adjacent cells. In animal models of sepsis-caused AKI, cortical changes underlie the acute injury. Notwithstanding these spatial differences in the kidneys' cellular and molecular architecture, the whole-organ gene expression profiling revealed that 33% of the 16,858 analyzed genes comprising the kidneys transcriptome were upregulated or downregulated in response to experimental sepsis (FIG. 3).

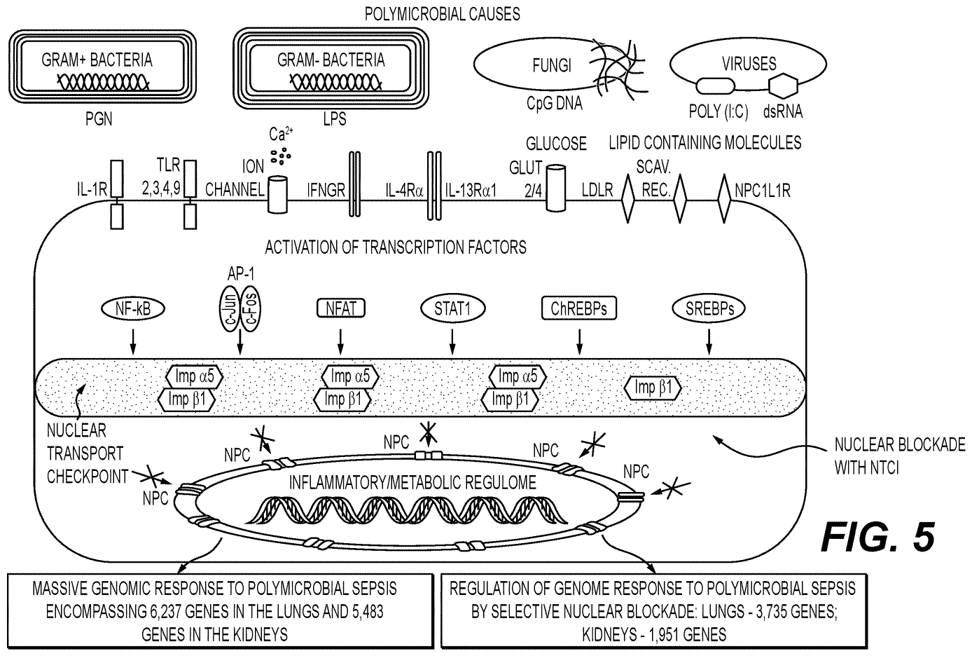

The selective nuclear blockade imposed by the NTCI simultaneously targets two nuclear transport shuttles, cytoplasmic adaptor proteins termed importin α5 and importin β1. These two cytoplasmic proteins are required for the nuclear translocation of an entire set of proinflammatory SRTFs and MTFs to the cell's nucleus. Thus, the NTCI disables the inflammatory and metabolic regulomes thereby silencing (until this study) at least 32 known genes encoding the mediators of inflammation in animal models of allergic, autoimmune, metabolic, microbial, and physical inflammation. In the experimental sepsis model studied by us herein, the number of genes regulated by the selective nuclear blockade in the lungs and kidneys exceeds 3,700 and 1,900, respectively (FIG. 5).

The selectivity of the nuclear blockade imposed by disabling importin α5- and importin β1-based nuclear transport of SRTFs and MTFs allows other members of importins α family to continue their nuclear translocation function. Moreover, the adverse impact of inhibiting importin α5 was allied by the report that mice with an importin α5 deficiency are viable and fertile and do not display any apparent morphological and behavioral defects. Significantly, the NTCI inhibits the nuclear translocation of larger transcription factors (MW>45 kDa), such as SRTFs and MTFs by targeting the Imp α5/Imp β1 complex. Therefore, in the presence of the NTCI, the smaller transcription factors (MW<45 kDa), which are essential to cell survival and maintenance, can freely translocate to the nucleus and contribute to the homeostasis and lifespan of cells treated with the NTCI. We found that the NTCI treatment did not alter the expression of the five housekeeping genes (Gusb, Hprt1, Hsp90ab1, Gapdh, and Actb).

The new generation cell-penetrating NTCI, the cSN50.1 peptide, regulated the massive genomic response to sepsis and enhanced bacterial clearance without institution of antimicrobial therapy, as documented here. In the experimental model of sepsis, the same NTCI increased the survival of mice from 30% in the antibiotic-treated control group to 55% in the group treated with the NTCI combined with antimicrobial therapy. Another experimental sepsis study using a cecal ligation and puncture model, independently showed that the first generation NTCI (SN50 peptide), combined with antimicrobial therapy, also improved survival. Moreover, the innate immunity reprogramming action of the NTCI (SN50 peptide) was subsequently showed by others in the adjuvant-mediated enhancement of response to the viral and bacterial vaccines. Notably, a new generation NTCI (the cSN50.1 peptide) used herein is 8 times more water soluble and bioavailable than the first generation NTCI (SN50 peptide) used in most of the studies.

The NTCI reported in this and earlier reports, represents a new class of broad-spectrum anti-inflammatory agents for topical (localized) genomic control of inflammation and, if needed, systemic therapy of sepsis and related diseases mediated by microbial inflammation. Other broad-spectrum anti-inflammatory agents, the glucocorticoids, e.g., dexamethasone, target the inflammatory regulome via the action of the cognate nuclear receptor, which functions as a transcription factor, inducing genomic response. Inadvertently, this response causes immunosuppression and dysregulated metabolism (hyperglycemia, hyperlipidemia) as well as skin atrophy, osteoporosis and atherosclerosis. In striking contrast, the NTCI reduces blood glucose, cholesterol, and triglycerides, and atherosclerosis while increasing the innate immunity-mediated clearance of bacteria in the blood, spleen, and lungs (FIG. 4).

The microbial clearance, operational in the blood, spleen, lungs, and other organs, reduces the dissemination of bacteria from an initial site of sepsis-causing infection. This primarily phagocytic process, mediated by circulating myelomonocytic cells (granulocytes and monocytes) and tissue macrophages, also depends on well-functioning small blood vessels (<100 μm diameter). Their normal function in the lungs, heart, kidneys, and other organs is essential for trafficking and extravasation of phagocytic and non-phagocytic immune cells. Microbial agents, joined by the host-produced mediators of innate immunity and inflammation, damage the small blood vessels.

These considerations are important as the wide-spread emergence of microbes that can escape antimicrobial therapy calls for new approaches to cytoprotective treatments of severe infections evolving into sepsis. Among the cytoprotective approaches, pathogen-directed monoclonal antibodies tested in Ebola virus-associated sepsis and other viral infections, recombinant SLIT protein, and NTCIs, await further preclinical and clinical studies. Frustratingly, over 100 published clinical trials in sepsis targeting microbial factors, cytokines (including anti-TNF-alpha and anti-IL-1 beta monoclonal antibodies), and blood coagulation regulators have thus far failed. A follow-up review analyzed more than 200 unique putative biomarkers of sepsis that have been proposed and concluded that none of these is used to guide therapies that target the host response.

The selective nuclear blockade that impedes the access of at least 7 families of transcription factors to the inflammatory and metabolic regulomes in the cell's nucleus (FIG. 5) controls cumulatively the expression of over 4,200 genes in the two major target organs in sepsis as documented herein. Moreover, it enhances the innate immunity-mediated bacterial clearance in the blood, spleen, and lungs (FIG. 4) and increases survival when combined with anti-microbial therapy. These gains are imperative as sepsis-causing infections are increasingly difficult to control due to growing multi-drug resistance. For example, community-acquired Methicillin-Resistant Staphylococcus aureus (MRSA) almost doubled the number of influenza deaths.

The upregulated and downregulated genes in sepsis can be categorized in the following functional groups: A. The mediators of innate immunity and inflammation (cytokines, chemokines, growth factors, cell-adhesion molecules, intracellular signaling intermediates, microbicidal peptides). This set also encompasses genes responsible for the inborn errors of immunity based on the 320 different gene defects underlying 330 distinct disorders as reported in 2018. B. The genes involved in the metabolism of cholesterol, triglycerides, fatty acids, and carbohydrates. C. The genes encoding transcription factors, including those participating in the feed-forward proinflammatory loop (e.g., cFOS), and metabolic signaling loops (e.g., SREBPs). D. The genes encoding physiologic suppressors of inflammation (e.g., SOCS1, SOCS3, CRADD, and A20). Since microvascular endothelial cells are dysfunctional during sepsis, we emphasize a separate category (E) for the genes encoding the endothelial regulatory signaling axis comprising angiopoietin-1 (angpt-1) and Tie2 and others. Microvascular endothelial injury plays a critically important role in the sepsis-induced dysfunction of the lungs, kidneys, and other organs.

Our genomic findings in experimental sepsis controlled by the selective nuclear blockade have significant translational implications for the mechanism of human sepsis and related complications. For example, the Intensive Care Unit-Acquired Weakness (ICUAW), that delays patients' recovery is mediated by Saa1 gene-encoded Serum Amyloid A1 contributing to the myotube injury. In addition, we identified in experimental sepsis another sepsis-suppressed gene linked to muscle dystrophy, anoctamin 5 (Ano5), and discovered its beneficial regulation by the NTCI, Importantly, the treatment with the NTCI lowered the expression of Saa1 in the lungs by 2.7-fold. Furthermore, it lowered Saa3 gene expression four-fold in the lungs and two-fold in the kidneys and raised 5-fold the expression of Anoctamin 5 gene (Tables 1 and 3).

Disseminated intravascular coagulation (DIC), is one of the major hematologic complications of sepsis. The NTCI treatment reduced the expression of the genes encoding fibrinogen chains and Factor X in the lungs and kidneys thereby reducing the risk of DIC in sepsis.

Metabolic derangements in human sepsis exemplified by elevated lactate level in blood and alteration in fatty acid metabolism were elevated in non-survivors of Septic Shock. Our study found strikingly profound changes in genes encoding endocrine and metabolic regulators. Hence, the G protein-coupled bile acid receptor-1 (Gpbar1 aka Takeda G protein-coupled receptor-5 [TGR5]) was downregulated in experimental sepsis. TGR5 stimulates glucagon-like peptide-1 (GLP-1) secretion to improve insulin sensitivity and hepatic metabolism. Significantly, the treatment with the NTCI increased 4-fold the expression of the Gpbar1. This underscores the NTCI's ability for metabolic improvement in sepsis and other severe infectious diseases. The two genes encoding cytochrome P450, family 11 subfamily b and a polypeptide 1, were most profoundly suppressed in experimental sepsis. The cytochrome P450 family 17 subfamily A member 1 participates in steroidogenesis induced by endothelin-1. Sepsis-induced suppression of these genes heralds potential failure of physiologic steroidogenesis. The NTCI partially reversed precipitous decline of these metabolic genes. The last but not least example of translational value emerging from our study is the gene encoding β-Klotho. It was downregulated by sepsis in the lungs and kidneys and increased by the NTCI. The β-Klotho is the essential component of fibroblast growth factor (FGF) receptor complexes required for high-affinity binding of endocrine FGF19 and FGF21. They evoke the signaling cascade actively involved in homeostatic maintenance of glucose metabolism and energy expenditure that are dysregulated in sepsis.

In summary, we uncovered the hitherto unreported induction of thousands of genes by experimental sepsis in the lungs and kidneys, the two major organs, which are the sites of acute respiratory distress syndrome (ARDS) and acute kidney injury (AKI), respectively. We also found that this genomic response to sepsis is regulated by the selective nuclear blockade with the NTCI. The resulting reprogramming of the genes in the lungs, spleen, and blood contributes to the increased bacterial clearance reducing the dissemination of polymicrobial infection (FIG. 4) and increasing survival in sepsis. Therefore, the selective nuclear blockade counteracts the action of the microbial virulence factors combined with the host's produced mediators of inflammation thereby protecting the major organs from potentially lethal septic injury.

Altogether, our results are of significant relevance to millions of individuals in all age groups threatened by sepsis. We submit that the NTCI-based approach paves the way for the effective treatment of sepsis, one of the most prevailing and challenging complication of severe infections worldwide. Our findings of robust genomic response to experimental sepsis and its successful control in the two major organs, lungs and kidneys, by the NTCI has valuable medical use. The NTCI (cSN50.1 aka. AMTX-100 CF) is in the ongoing Phase 2 clinical trial for mild to moderate Atopic Dermatitis (NCT04313400), the most common skin disease.

### D. References

Abreu A P, Trarbach E B, de Castro M, Frade Costa E M, Versiani B, Matias Baptista M T, et al. Loss-of-Function Mutations in the Genes Encoding Prokineticin-2 or Prokineticin Receptor-2 Cause Autosomal Recessive Kallmann Syndrome. The Journal of Clinical Endocrinology & Metabolism (2008) 93 (10): 4113-8. doi: 10.1210/jc.2008-0958.
Angus D C, van der Poll T. Severe Sepsis and Septic Shock. The New England journal of medicine (2013) 369 (9): 840-51. Epub 2013 Aug. 30. doi: 10.1056/NEJMra1208623.
Bambouskova M, Gorvel L, Lampropoulou V, Sergushichev A, Loginicheva E, Johnson K, et al. Electrophilic Properties of Itaconate and Derivatives Regulate the Ikappabzeta-Atf3 Inflammatory Axis. Nature (2018) 556 (7702): 501-4. Epub 2018 Apr. 20. doi: 10.1038/s41586-018-0052-z.
Bousfiha A, Jeddane L, Picard C, Ailal F, Bobby Gaspar H, Al-Herz W, et al. The 2017 luis Phenotypic Classification for Primary Immunodeficiencies. J Clin Immunol (2018) 38 (1): 129-43. Epub 2017 Dec. 12. doi: 10.1007/s10875-017-0465-8.
Caruso R, Warner N, Inohara N, Nunez G. Nod1 and Nod2: Signaling, Host Defense, and Inflammatory Disease. Immunity (2014) 41 (6): 898-908. Epub 2014 Dec. 20. doi: 10.1016/j.immuni.2014.12.010.
Chang T Y, Li B L, Chang C C, Urano Y. Acyl-Coenzyme A: Cholesterol Acyltransferases. Am J Physiol Endocrinol Metab (2009) 297 (1): E1-9. Epub 2009 Jan. 15. doi: 10.1152/ajpendo.90926.2008.
Chiusa M, Hu W, Zienkiewicz J, Chen X, Zhang M Z, Harris R C, et al. Egf Receptor-Mediated Fus Phosphorylation Promotes Its Nuclear Translocation and Fibrotic Signaling. The Journal of cell biology (2020) 219 (9). Epub 2020 Jul. 18. doi: 10.1083/jcb.202001120.
Chung D R, Huh K. Novel Pandemic Influenza a (H1n1) and Community-Associated Methicillin-Resistant Staphylococcus Aureus Pneumonia. Expert Rev Anti Infect Ther (2015) 13 (2): 197-207. Epub 2015 Jan. 13. doi: 10.1586/14787210.2015.999668.
Cohen D M, Steger D J. Nuclear Receptor Function through Genomics: Lessons from the Glucocorticoid Receptor. Trends in endocrinology and metabolism: TEM (2017) 28 (7): 531-40. Epub 2017 May 13. doi: 10.1016/j.tem.2017.04.001.
Cordes T, Wallace M, Michelucci A, Divakaruni A S, Sapcariu S C, Sousa C, et al. Immunoresponsive Gene 1 and Itaconate Inhibit Succinate Dehydrogenase to Modulate Intracellular Succinate Levels. The Journal of biological chemistry (2016) 291 (27): 14274-84. Epub 2016 May 18. doi: 10.1074/jbc.M115.685792.
Diamos A G, Hunter J G L, Pardhe M D, Rosenthal S H, Sun H, Foster B C, et al. High Level Production of Monoclonal Antibodies Using an Optimized Plant Expression System. Front Bioeng Biotechnol (2019) 7:472. Epub 2020 Feb. 6. doi: 10.3389/fbioe.2019.00472.
DiGiandomenico A, Veach R A, Zienkiewicz J, Moore D J, Wylezinski L S, Hutchens M A, et al. The “Genomic Storm” Induced by Bacterial Endotoxin Is Calmed by a Nuclear Transport Modifier That Attenuates Localized and Systemic Inflammation. PloS one (2014) 9 (10): e110183. Epub 2014 Oct. 21. doi: 10.1371/journal.pone.0110183.
Freedman N D, Yamamoto K R. Importin 7 and Importin Alpha/Importin Beta Are Nuclear Import Receptors for the Glucocorticoid Receptor. Molecular biology of the cell (2004) 15(5): 2276-86. Epub 2004 Mar. 9. doi: 10.1091/mbc.E03-11-0839.
Gallo C G, Fiorino S, Posabella G, Antonacci D, Tropeano A, Pausini E, et al. Covid-19, What Could Sepsis, Severe Acute Pancreatitis, Gender Differences, and Aging Teach Us? Cytokine (2021) 148:155628. Epub 2021 Aug. 20. doi: 10.1016/j.cyto.2021.155628.
Gogos C A, Lekkou A, Papageorgiou O, Siagris D, Skoutelis A, Bassaris H P. Clinical Prognostic Markers in Patients with Severe Sepsis: A Prospective Analysis of 139 Consecutive Cases. J Infect (2003) 47 (4): 300-6. Epub 2003 Oct. 15. doi: 10.1016/s0163-4453 (03) 00101-4.
Hahn A, Kny M, Pablo-Tortola C, Todiras M, Willenbrock M, Schmidt S, et al. Serum Amyloid A1 Mediates Myotube Atrophy Via Toll-Like Receptors. J Cachexia Sarcopenia Muscle (2020) 11 (1): 103-19. Epub 2019 Aug. 24. doi: 10.1002/jcsm.12491.
Hawiger J, Musser J M. How to Approach Genome Wars in Sepsis? Crit Care (2011) 15 (6): 1007. Epub 2011 Dec. 6. doi: 10.1186/cc10482.
Hawiger J, Veach R A, Zienkiewicz J. New Paradigms in Sepsis: From Prevention to Protection of Failing Microcirculation. Journal of thrombosis and haemostasis: JTH (2015) 13 (10): 1743-56. Epub 2015 Jul. 21. doi: 10.1111/jth.13061.
Hawiger J, Zienkiewicz J. Decoding Inflammation, Its Causes, Genomic Responses, and Emerging Countermeasures. Scand J Immunol (2019): e12812. Epub 2019 Aug. 6. doi: 10.1111/sji.12812.
Higgins S J, De Ceunynck K, Kellum J A, Chen X, Gu X, Chaudhry S A, et al. Tie2 Protects the Vasculature against Thrombus Formation in Systemic Inflammation. The Journal of clinical investigation (2018) 128 (4): 1471-84. Epub 2018 Jan. 24. doi: 10.1172/JCI97488.
Hinze C, Karaiskos N, Boltengagen A, Walentin K, Redo K, Himmerkus N, et al. Kidney Single-Cell Transcriptomes Predict Spatial Corticomedullary Gene Expression and Tissue Osmolality Gradients. J Am Soc Nephrol (2021) 32 (2): 291-306. Epub 2020 Nov. 27. doi: 10.1681/ASN.2020070930.
Hoebe K, Jiang Z, Tabeta K, Du X, Georgel P, Crozat K, et al. Genetic Analysis of Innate Immunity. Advances in immunology (2006) 91:175-226. Epub 2006 Aug. 30. doi: 10.1016/S0065-2776 (06) 91005-0.
Hogan B L, Barkauskas C E, Chapman H A, Epstein J A, Jain R, Hsia C C, et al. Repair and Regeneration of the Respiratory System: Complexity, Plasticity, and Mechanisms of Lung Stem Cell Function. Cell Stem Cell (2014) 15 (2): 123-38. Epub 2014 Aug. 12. doi: 10.1016/j.stem.2014.07.012.
Hu Q, Hao C, Tang S. From Sepsis to Acute Respiratory Distress Syndrome (Ards): Emerging Preventive Strategies Based on Molecular and Genetic Researches. Biosci Rep (2020) 40 (5). Epub 2020 Apr. 23. doi: 10.1042/BSR20200830.
Hua S, Liu Q, Li J, Fan M, Yan K, Ye D. Beta-Klotho in Type 2 Diabetes Mellitus: From Pathophysiology to Therapeutic Strategies. Rev Endocr Metab Disord (2021) 22 (4): 1091-109. Epub 2021 Jun. 14. doi: 10.1007/s11154-021-09661-1.
Islam M B, Chowdhury U N, Nain Z, Uddin S, Ahmed M B, Moni M A. Identifying Molecular Insight of Synergistic Complexities for Sars-Cov-2 Infection with Pre-Existing Type 2 Diabetes. Comput Biol Med (2021) 136:104668. Epub 2021 Aug. 3. doi: 10.1016/j.compbiomed.2021.104668.
Iwashyna T J, Ely E W, Smith D M, Langa K M. Long-Term Cognitive Impairment and Functional Disability among Survivors of Severe Sepsis. Jama (2010) 304 (16): 1787-94. Epub 2010 Oct. 28. doi: 10.1001/jama.2010.1553.
Jurek B, Neumann I D. The Oxytocin Receptor: From Intracellular Signaling to Behavior. Physiological reviews (2018) 98 (3): 1805-908. Epub 2018 Jun. 14. doi: 10.1152/physrev.00031.2017.
Kalbak K. Incidence of Arteriosclerosis in Patients with Rheumatoid Arthritis Receiving Long-Term Corticosteroid Therapy. Ann Rheum Dis (1972) 31 (3): 196-200. Epub 1972 May 1. doi: 10.1136/ard.31.3.196.
Kanhutu K, Jones P, Cheng A C, Grannell L, Best E, Spelman D. Spleen Australia Guidelines for the Prevention of Sepsis in Patients with Asplenia and Hyposplenism in Australia and New Zealand. Intern Med J (2017) 47 (8): 848-55. Epub 2016 Dec. 8. doi: 10.1111/imj.13348.
Kim W Y, Hong S B. Sepsis and Acute Respiratory Distress Syndrome: Recent Update. Tuberc Respir Dis (Seoul) (2016) 79 (2): 53-7. Epub 2016 Apr. 12. doi: 10.4046/trd.2016.79.2.53.
Kinjo T, Higashi H, Uno K, Kuramoto N. Apelin/Apelin Receptor System: Molecular Characteristics, Physiological Roles, and Prospects as a Target for Disease Prevention and Pharmacotherapy. Curr Mol Pharmacol (2021) 14 (2): 210-9. Epub 2020 Jun. 3. doi: 10.2174/1874467213666200602133032.
Kramarow E A. Sepsis-Related Mortality among Adults Aged 65 and Over: United States, 2019. NCHS Data Brief (2021) (422): 1-8. Epub 2021 Nov. 30.
Langley R J, Tsalik E L, van Velkinburgh J C, Glickman S W, Rice B J, Wang C, et al. An Integrated Clinico-Metabolomic Model Improves Prediction of Death in Sepsis. Sci Transl Med (2013) 5 (195): 195ra95. Epub 2013 Jul. 26. doi: 10.1126/scitranslmed.3005893.
Lee S J, Sekimoto T, Yamashita E, Nagoshi E, Nakagawa A, Imamoto N, et al. The Structure of Importin-Beta Bound to Srebp-2: Nuclear Import of a Transcription Factor. Science (2003) 302 (5650): 1571-5. Epub 2003 Dec. 3. doi: 10.1126/science.1088372.
Liu D, Li C, Chen Y, Burnett C, Liu X Y, Downs S, et al. Nuclear Import of Proinflammatory Transcription Factors Is Required for Massive Liver Apoptosis Induced by Bacterial Lipopolysaccharide. The Journal of biological chemistry (2004) 279 (46): 48434-42. Epub 2004 Sep. 4. doi: 10.1074/jbc.M407190200.
Liu D, Zienkiewicz J, DiGiandomenico A, Hawiger J. Suppression of Acute Lung Inflammation by Intracellular Peptide Delivery of a Nuclear Import Inhibitor. Molecular therapy: the journal of the American Society of Gene Therapy (2009) 17 (5): 796-802. Epub 2009 Mar. 5. doi: 10.1038/mt.2009.18.
Liu Y, Major A S, Zienkiewicz J, Gabriel C L, Veach R A, Moore D J, et al. Nuclear Transport Modulation Reduces Hypercholesterolemia, Atherosclerosis, and Fatty Liver. Journal of the American Heart Association (2013) 2 (2): e000093. Epub 2013 Apr. 9. doi: 10.1161/JAHA.113.000093.
Liu Y, Zienkiewicz J, Boyd K L, Smith T E, Xu Z Q, Hawiger J. Hyperlipidemic Hypersensitivity to Lethal Microbial Inflammation and Its Reversal by Selective Targeting of Nuclear Transport Shuttles. Scientific reports (2021) 11 (1): 11907. Epub 2021 Jun. 9. doi: 10.1038/s41598-021-91395-w.
Liu Y, Zienkiewicz J, Qiao H, Gibson-Corley K N, Boyd K L, Veach R A, et al. Genomic Control of Inflammation in Experimental Atopic Dermatitis. Scientific reports (2022) 12 (1): 18891. Epub 2022 Nov. 8. doi: 10.1038/s41598-022-23042-x.
London N R, Zhu W, Bozza F A, Smith M C, Greif D M, Sorensen L K, et al. Targeting Robo4-Dependent Slit Signaling to Survive the Cytokine Storm in Sepsis and Influenza. Science translational medicine (2010) 2 (23): 23ra19. Epub 2010 Apr. 9. doi: 10.1126/scitranslmed.3000678.
Luo Z, Lei H, Sun Y, Liu X, Su D F. Orosomucoid, an Acute Response Protein with Multiple Modulating Activities. J Physiol Biochem (2015) 71 (2): 329-40. Epub 2015 Feb. 26. doi: 10.1007/s13105-015-0389-9.
Marshall J C, Leligdowicz A. Gaps and Opportunities in Sepsis Translational Research. EBioMedicine (2022) 86:104387. Epub 2022 Dec. 6. doi: 10.1016/j.ebiom.2022.104387.
Marshall J C. Why Have Clinical Trials in Sepsis Failed? Trends Mol Med (2014) 20 (4): 195-203. Epub 2014 Mar. 4. doi: 10.1016/j.molmed.2014.01.007.
McNeil B D, Pundir P, Meeker S, Han L, Undem B J, Kulka M, et al. Identification of a Mast-Cell-Specific Receptor Crucial for Pseudo-Allergic Drug Reactions. Nature (2015) 519 (7542): 237-41. Epub 2014 Dec. 18. doi: 10.1038/nature14022.
Mills E L, Pierce K A, Jedrychowski M P, Garrity R, Winther S, Vidoni S, et al. Accumulation of Succinate Controls Activation of Adipose Tissue Thermogenesis. Nature (2018) 560 (7716): 102-6. Epub 2018 Jul. 20. doi: 10.1038/s41586-018-0353-2.
Mills E L, Ryan D G, Prag H A, Dikovskaya D, Menon D, Zaslona Z, et al. Itaconate Is an Anti-Inflammatory Metabolite That Activates Nrf2 Via Alkylation of Keap1. Nature (2018) 556 (7699): 113-7. Epub 2018 Mar. 29. doi: 10.1038/nature25986.
Molema G, Zijlstra J G, van Meurs M, Kamps J. Renal Microvascular Endothelial Cell Responses in Sepsis-Induced Acute Kidney Injury. Nat Rev Nephrol (2022) 18 (2): 95-112. Epub 2021 Oct. 21. doi: 10.1038/s41581-021-00489-1.
Moser B A, Steinhardt R C, Escalante-Buendia Y, Boltz D A, Barker K M, Cassaidy B J, et al. Increased Vaccine Tolerability and Protection Via Nf-Kappab Modulation. Sci Adv (2020) 6 (37). Epub 2020 Sep. 13. doi: 10.1126/sciadv.aaz8700.
O'Sullivan A W, Wang J H, Redmond H P. Nf-Kappab and P38 Mapk Inhibition Improve Survival in Endotoxin Shock and in a Cecal Ligation and Puncture Model of Sepsis in Combination with Antibiotic Therapy. J Surg Res (2009) 152 (1): 46-53. Epub 2008 Nov. 26. doi: 10.1016/j.jss.2008.04.030.
Paoli C J, Reynolds M A, Sinha M, Gitlin M, Crouser E. Epidemiology and Costs of Sepsis in the United States—an Analysis Based on Timing of Diagnosis and Severity Level. Crit Care Med (2018) 46 (12): 1889-97. Epub 2018 Jul. 27. doi: 10.1097/CCM.0000000000003342.
Pathak P, Xie C, Nichols R G, Ferrell J M, Boehme S, Krausz K W, et al. Intestine Farnesoid X Receptor Agonist and the Gut Microbiota Activate G-Protein Bile Acid Receptor-1 Signaling to Improve Metabolism. Hepatology (2018) 68 (4): 1574-88. Epub 2018 Feb. 28. doi: 10.1002/hep.29857.
Percopo C M, Krumholz J O, Fischer E R, Kraemer L S, Ma M, Laky K, et al. Impact of Eosinophil-Peroxidase (Epx) Deficiency on Eosinophil Structure and Function in Mouse Airways. J Leukoc Biol (2019) 105 (1): 151-61. Epub 2018 Oct. 5. doi: 10.1002/jlb.3ab0318-090rr.
Perlmann T. Glucocorticoid Receptor DNA-Binding Specificity Is Increased by the Organization of DNA in Nucleosomes. Proceedings of the National Academy of Sciences of the United States of America (1992) 89 (9): 3884-8. Epub 1992 May 1.
Qiao, H., Zienkiewicz, J., Liu, Y., and Hawiger, J. (2023) Frontiers in Immunology 14:1221102.
Radjabova V, Mastroeni P, Skjodt K, Zaccone P, de Bono B, Goodall J C, et al. Tarm1 Is a Novel Leukocyte Receptor Complex-Encoded Itam Receptor That Costimulates Proinflammatory Cytokine Secretion by Macrophages and Neutrophils. J Immunol (2015) 195 (7): 3149-59. Epub 2015 Aug. 28. doi: 10.4049/jimmunol.1401847.
Rudd K E, Johnson S C, Agesa K M, Shackelford K A, Tsoi D, Kievlan D R, et al. Global, Regional, and National Sepsis Incidence and Mortality, 1990-2017: Analysis for the Global Burden of Disease Study. Lancet (2020) 395 (10219): 200-11. Epub 2020 Jan. 20. doi: 10.1016/S0140-6736 (19) 32989-7.
Schulze Blasum B, Schröter R, Neugebauer U, Hofschröer V, Pavenstädt H, Ciarimboli G, et al. The Kidney-Specific Expression of Genes Can Be Modulated by the Extracellular Osmolality. Faseb j (2016) 30 (10): 3588-97. Epub 2016 Jul. 29. doi: 10.1096/fj.201600319R.
Shi C, Pamer E G. Monocyte Recruitment During Infection and Inflammation. Nature reviews Immunology (2011) 11 (11): 762-74. Epub 2011 Oct. 11. doi: 10.1038/nri3070.
Shmidt T, Hampich F, Ridders M, Schultrich S, Hans V H, Tenner K, et al. Normal Brain Development in Importin-Alpha5 Deficient-Mice. Nature cell biology (2007) 9 (12): 1337-8; author reply 9. Epub 2007 Dec. 7. doi: 10.1038/ncb1207-1337.
Soontrapa P, Liewluck T. Anoctamin 5 (Ano5) Muscle Disorders: A Narrative Review. Genes (Basel) (2022) 13 (10). Epub 2022 Oct. 28. doi: 10.3390/genes13101736.
Stevens S M, Woller S C, Bauer K A, Kasthuri R, Cushman M, Streiff M, et al. Guidance for the Evaluation and Treatment of Hereditary and Acquired Thrombophilia. J Thromb Thrombolysis (2016) 41 (1): 154-64. Epub 2016 Jan. 19. doi: 10.1007/s11239-015-1316-1.
Theodore M, Kawai Y, Yang J, Kleshchenko Y, Reddy S P, Villalta F, et al. Multiple Nuclear Localization Signals Function in the Nuclear Import of the Transcription Factor Nrf2. The Journal of biological chemistry (2008) 283 (14): 8984-94. Epub 2008 Feb. 2. doi: 10.1074/jbc.M709040200.
Torgerson T R, Colosia A D, Donahue J P, Lin Y Z, Hawiger J. Regulation of Nf-Kappa B, Ap-1, Nfat, and Stat1 Nuclear Import in T Lymphocytes by Noninvasive Delivery of Peptide Carrying the Nuclear Localization Sequence of Nf-Kappa B P50. J Immunol (1998) 161 (11): 6084-92. Epub 1998 Dec. 2.
Torres M J, López-Moncada F, Herrera D, Indo S, Lefian A, Llanos P, et al. Endothelin-1 Induces Changes in the Expression Levels of Steroidogenic Enzymes and Increases Androgen Receptor and Testosterone Production in the Pc3 Prostate Cancer Cell Line. Oncol Rep (2021) 46 (2). Epub 2021 Jun. 25. doi: 10.3892/or.2021.8122.
Tsao C W, Aday A W, Almarzooq Z I, Alonso A, Beaton A Z, Bittencourt M S, et al. Heart Disease and Stroke Statistics-2022 Update: A Report from the American Heart Association. Circulation (2022) 145 (8): e153-e639. Epub 2022 Jan. 27. doi: 10.1161/CIR.0000000000001052.
Veach R A, Liu D, Yao S, Chen Y, Liu X Y, Downs S, et al. Receptor/Transporter-Independent Targeting of Functional Peptides across the Plasma Membrane. The Journal of biological chemistry (2004) 279 (12): 11425-31. Epub 2003 Dec. 31. doi: 10.1074/jbc.M311089200.
Veach R A, Liu Y, Zienkiewicz J, Wylezinski L S, Boyd K L, Wynn J L, et al. Survival, Bacterial Clearance and Thrombocytopenia Are Improved in Polymicrobial Sepsis by Targeting Nuclear Transport Shuttles. PloS one (2017) 12 (6): e0179468. Epub 2017 Jun. 20. doi: 10.1371/journal.pone.0179468.
Vincent J L, Rello J, Marshall J, Silva E, Anzueto A, Martin C D, et al. International Study of the Prevalence and Outcomes of Infection in Intensive Care Units. Jama (2009) 302 (21): 2323-9. Epub 2009 Dec. 3. doi: 10.1001/jama.2009.1754.
Why Mouse Matters: Human Genome Research Institute (NHGRI) (2010). Available from: https://www.genome.gov/10001343/2000-release-mouse-sequencing-consortium.
Wong G, Kobinger G P. Backs against the Wall: Novel and Existing Strategies Used During the 2014-2015 Ebola Virus Outbreak. Clin Microbiol Rev (2015) 28 (3): 593-601. doi: 10.1128/cmr.00014-15.
Wong H R, Shanley T P, Sakthivel B, Cvijanovich N, Lin R, Allen G L, et al. Genome-Level Expression Profiles in Pediatric Septic Shock Indicate a Role for Altered Zinc Homeostasis in Poor Outcome. Physiol Genomics (2007) 30 (2): 146-55. Epub 2007 Mar. 22. doi: 10.1152/physiolgenomics.00024.2007.
Wu T, Xu F, Su C, Li H, Lv N, Liu Y, et al. Alterations in the Gut Microbiome and Cecal Metabolome During Klebsiella Pneumoniae-Induced Pneumosepsis. Frontiers in immunology (2020) 11:1331. Epub 2020 Aug. 28. doi: 10.3389/fimmu.2020.01331.
Wynn J L, Cvijanovich N Z, Allen G L, Thomas N J, Freishtat R J, Anas N, et al. The Influence of Developmental Age on the Early Transcriptomic Response of Children with Septic Shock. Mol Med (2011) 17 (11-12): 1146-56. Epub 2011 Jul. 9. doi: 10.2119/molmed.2011.00169.
Wynn J L, Scumpia P O, Delano M J, O'Malley K A, Ungaro R, Abouhamze A, et al. Increased Mortality and Altered Immunity in Neonatal Sepsis Produced by Generalized Peritonitis. Shock (2007) 28 (6): 675-83. Epub 2007 Jul. 11. doi: 10.1097/SHK.0b013e3180556d09.
Wynn J L, Wilson C S, Hawiger J, Scumpia P O, Marshall A F, Liu J H, et al. Targeting Il-17a Attenuates Neonatal Sepsis Mortality Induced by Il-18. Proceedings of the National Academy of Sciences of the United States of America (2016) 113 (19): E2627-35. Epub 2016 Apr. 27. doi: 10.1073/pnas.1515793113.
Xin A, Zhao Y, Yu H, Shi H, Diao H, Zhang Y. Characterization of B-Defensin 42 Expressed in Principal Cells at the Initial Segment of the Rat Epididymis. Acta Biochimica et Biophysica Sinica (2015) 47 (11): 861-9. doi: 10.1093/abbs/gmv089.
Zhou W, Zhang C, Zhuang Z, Zhang J, Zhong C. Identification of Two Robust Subclasses of Sepsis with Both Prognostic and Therapeutic Values Based on Machine Learning Analysis. Frontiers in immunology (2022) 13:1040286. Epub 2022 Dec. 13. doi: 10.3389/fimmu.2022.1040286.
Zienkiewicz J, Armitage A, Hawiger J. Targeting Nuclear Import Shuttles, Importins/Karyopherins Alpha by a Peptide Mimicking the Nfkappab1/P50 Nuclear Localization Sequence. Journal of the American Heart Association (2013) 2 (5): e000386. Epub 2013 Sep. 18. doi: 10.1161/JAHA.113.000386.

1. A method of reversing the expression profile of one or more genes encoding mediators of inflammation in a subject whose expression modulates during microbial inflammation as a result of microbial infection comprising administering to the subject a therapeutically effective amount of a nuclear transport checkpoint inhibitor (NTCI).

2. The method of claim 1, wherein the one or more genes are selected from the group consisting of I110, Acod1, Saa3, Gpr31b, Fgf23, Igfbp1, Mrgpra2b, Ccl3, Ccl4, Gpr84, Adamts4, Madcam1, Olfm4, Il1f9, Gpr31a, Gpr31c, Ptx3, Slc7a11, Gm6553, Tmprss11g, Tac1, Il1f6, Ccl2, Psg16, Fosl1, Sprr2a2, Serpina3m, Il1rn, Cc120, Tnf, Gm43181, Clec4e, Gm45774, Ugtla10, Kcnt1, Mt1, Mefv, Clec4d, Ms4a4a, Btbd16, Spic, Orm1, Serpina3f, Il1b, Serpina10, 9830107B12Rik, Gm49839, Msr1, Ankrd2, Saa4, S100a8, Hpx, Apoa4, Steap4, Cxcl10, S100a9, Wfdc17, Gnat1, Fgg, Fgb, Lilr4b, 2010003K11Rik, Serpina3n, AA467197, Gm16685, Timp1, Fga, Ifitm6, Lcn2, F10, Leap2, Retnlg, Ccl7, Ikbke, Vcan, F13a1, Serpina3g, 1200007C13Rik, Ngp, Siglece, Lrrc25, Gm10309, Trem1, Slc39a14, Fgl1, 1127, Tcp10b, Serpina3i, Cxc19, A730049H05Rik, Gm8883, Fpr2, Saa2, Saa1, Prok2, B230303A05Rik, Gm43079, Gm49383, Cldn14, Gm15056, U90926, Stfa2, Prokr2, Lbx1, Cstdc5, Tarm1, Mrgpra2a, Cstdc4, Gm48786, Gm19510, Gm32089, Gm5966, Stfa3, Tmem132e, Orm2, Gm28551, A630023A22Rik, Gm7457, Serpina7, Bmerb1, Klk9, 1700011B04Rik, Asprv1, Dmbt1, Tmprss11d, Slc5a7, Ifitm7, Gm38340, Pglyrp3, Gm26748, 4930431P03Rik, 4732490B 19Rik, Sectm1b, Gm4948, Gm20406, Ras110b, Gm36079, Sult2a3, Apcs, Mymk, Nkx2-9, Gm26535, Rnase2a, Slc22a20, Stfa211, Gm4949, Gm11738, Gm29686, Serpine3, Gm807, Gm26444, Trdv5, Sultle1, Serpina3e-ps, 4930578G10Rik, Gm37645, Ceacam19, Ackr1, Ms4a3, B130046B21Rik, Ctcflos, Doc2b, Gm49573, Gm38275, Tex44, Gpr22, Ceacam10, E030018B13Rik, Gm37131, Htr3a, Slc13a5, Nxf3, Gm49427, Pde6h, Ubd, Fcrlb, Sult2a5, Tat, Gm6610, Gm44292, Gm44165, Prss22, Gm11714, Gm49368, Gm48065, Aoah, Tctex1d1, Phox2a, Slc36a3, Sult2a1, Gm37347, Gm50083, Epx, Gm26965, Reg3g, Cela3b, Gm12503, Gpbar1, Dnah6, Fut2, Muc4, Gm43189, Angpt17, Pcdh8, Sftpa1, Mlana, Gm16350, Syt2, Gm17055, Ppy, Ccdc33, Gm33280, Gm45643, Lrit3, Sbk2, Slc6a19os, Slamf1, Gm5541, Fgf21, Gm49392, Slc6a16, Olr1, Gm35028, Slfn4, Gm20548, Gm40709, Speer4f2, Gm13363, Egr2, Gm44260, Marc1, Crygn, Lpo, Gabrp, Bpifc, Fgf18, Lncenc1, Prss27, Muc19, Gm39929, Gm2670, 5830487J09Rik, Olfr1442, Krt20, Gm19684, Gm33424, 4732419C18Rik, Gm37621, Creb313, Gm14319, Dnah10, Areg, Armc12, Eif4ebp3, Gdf6, Gm49037, Gm42793, Der13, Nptx2, Atp12a, Kcna7, Prg4, Ctla4, Gm44164, Mmp7, Gm44949, Slc17a8, Lamb3, Plekhs1, Trim17, Dusp8, 4930422C21Rik, Gm10727, Col27a1, Tacc2, Fat2, Lif, Klk1b1, Tsc22d1, Wnt8b, Grem1, Hesx1, Hhipl1, Ugtla5, Klk1b4, Gm8947, A530013C23Rik, Mylk4, Cyp2a5, Muc1, Gm44588, Gm45053, Ano5, Gsg11, Spon2, Oxtr, Rnf112, Ppp2r2c, Crabp1, St6galnac3, Agtr2, Gjc2, Gm33195, Gabra4, Aplnr, E030013I19Rik, Pcdh12, Chp2, Gng8, Pla2g2d, Gm20683, Mansc4, Gm10824, Ces2b, Kank4, Zmat4, Cdr1os, Wnt2, Gm17040, Wfdc16, Rab42, Tbxa2r, Plppr5, Misp3, Mmp28, Lrrn4, Sost, Tmem200a, Shisa2, Stmn2, Chrm1, Tmeff2, 2810433D01Rik, Pabpc41, Dact3, Mett124, Gpr34, D430019H16Rik, Hoxc5, Tekt5, Megf10, Wscd1, Gm32219, Tril, Agtr1b, 2610203C22Rik, Abcg3, Star, Grik2, Grin2c, Vstm5, Smad9, Pianp, Rnf144a, Cthrc1, Nxpe4, Mrc2, Cx3cr1, Il5ra, Kcnc4, Fam163a, Cxcr3, A330074K22Rik, Gm11627, Mmp11, Fam181b, Cd209b, Tnfrsf19, Sept4, Nlgn3, Baalc, Ngb, Nxph3, Klb, Igkv4-72, Ptprv, Nrep, Itga8, Myct1, Palm, Syt3, Tspan18, Piezo2, Nat8f3, Cngb1, Cd209g, Faim2, Gfra2, Srpk3, Gm3716, Klhl13, Mogat2, Tmem233, Duxb13, Rnase1, Defb42, AL935121.1, Zp1, Snurf, Gm25541, 4933403008Rik, 1810046K07Rik, Gm5639, Myh2, Gm765, Gm50242, Gm6018, Ackr4, Tnfrsf17, Gm37858, Igkv14-130, Ptprh, Gpr165, Gm47652, Gm16534, 0610012D04Rik, Gm36569, Gzmk, Gm9918, Gm33677, St6gal2, Krtap17-1, Olfr1342, Lhcgr, Ccdc42, Zfp985, Igkv4-70, Gm31517, Gm9767, Olfr71, Slc26a3, Trpv3, Gm49727, Gm43951, E330018M18Rik, Calcb, Rspo2, Glp1r, Fam124b, Amtn, 4930570G19Rik, Gm11574, Pla2g2e, Sfrp2, Pigz, Gm13688, Abca14, B530045E10Rik, Fras1, Gm12159, Gm48488, 9030404E10Rik, Hmcn1, Igkv4-68, Wnt7a, Tmem74, Ighv1-66, Cr2, Slc5a11, Lrrc17, Slc22a14, Dipk2b, Fgf18, Ndp, Gm17334, Btn12, Gm10432, Colq, Gm48662, 4833412C05Rik, Gm12576, Igkv6-17, Gm13816, Tmem266, Ighv5-6, Cntn4, Rbp7, Insyn2a, Slc5a12, Gm9947, Igkv12-89, Gm26795, Ddx4, Dclk3, Hpcal4, Ndnf, Igkv17-121, Slurp1, Ano4, Ighv1-7, Rimklb, Igkv4-80, Cyp11b1, Cyp11a1, Akr1cl, Hsd3b1, Scg2, Th, Cyp21a1, Chga, Cyp21a2-ps, Pnmt, Chgb, Cyp11b2, Caln1, Kcnk9, Dbh, Gm28382, Pla2g2f, Nr5a1, Pcskln, Mgarp, 4930486L24Rik, Cryba4, Atp2b3, Resp18, E330017L17Rik, Pcsk1, Snap25, Rims4, Slc24a2, Phox2b, Slc18a2, Eef1a2, Chrna3, Hand2os1, Cntnap5a, Rasgrf1, Hand2, Pnmal1, Necab2, Tbx20, Stmn3, Gm17322, St8sia3, Rab33a, Synpr, Rit2, Camkv, Gdap1, Soga3, Mdga2, Mab2112, Nrsn1, Cadps, Gnb3, 1520401A03Rik, Nrg3, Spock1, Cdk5r2, Ina, Act16b, Npy2r, Gal, Nrsn2, Gabrg2, Gpr158, Slc7a14, Sphkap, Tmem255a, 8430419K02Rik, Scg3, Gria1, Txndc2, Gm27477, Gria2, Gjd2, Lrrtm2, Hsd3b6, Diras1, Omg, Gm49391, Fam155a, Gm2007, Kcnq2, Gm42372, Is11, Lingo3, Prl8a2, Adh7, Lgi1, Gm47815, Gm14592, Cdx4, Marcks11-ps4, Sstr3, Pcdh10, Gm27000, Tub, Mc2r, Il20ra, Grin2a, IL-1b, IL-10, Tnf-a, Ifn-g, Nos1, Sele, Sell, Selp, Acat3, Tnfrsf1b, Csf1, Cx3cl1, and Ptgs2, Ccl12, Ccr2, Ccr5, Cxcl11, and/or Isg15.

3. The method of claim 2, wherein the expression of the one or more genes increases during microbial inflammation and wherein the one or more genes are selected from the group consisting of I110, Acod1, Saa3, Gpr31b, Fgf23, Igfbp1, Mrgpra2b, Ccl3, Ccl4, Gpr84, Adamts4, Madcam1, Olfm4, Il1f9, Gpr31a, Gpr31c, Ptx3, Slc7a11, Gm6553, Tmprss11g, Tac1, Il1f6, Ccl2, Psg16, Fosl1, Sprr2a2, Serpina3m, Il1rn, Cc120, Tnf, Gm43181, Clec4e, Gm45774, Ugtla10, Kcnt1, Mt1, Mefv, Clec4d, Ms4a4a, Btbd16, Spic, Orm1, Serpina3f, Il1b, Serpina10, 9830107B12Rik, Gm49839, Msr1, Ankrd2, Saa4, S100a8, Hpx, Apoa4, Steap4, Cxc110, S100a9, Wfdc17, Gnat1, Fgg, Fgb, Lilr4b, 2010003K11Rik, Serpina3n, AA467197, Gm16685, Timp1, Fga, Ifitm6, Lcn2, F10, Leap2, Retnlg, Ccl7, Ikbke, Vcan, F13a1, Serpina3g, 1200007C13Rik, Ngp, Siglece, Lrrc25, Gm10309, Trem1, Slc39a14, Fgl1, 1127, Tcp10b, Serpina3i, Cxc19, A730049H05Rik, Gm8883, Fpr2, Saa2, Saa1, Prok2, B230303A05Rik, Gm43079, Gm49383, Cldn14, Gm15056, U90926, Stfa2, Prokr2, Lbx1, Cstdc5, Tarm1, Mrgpra2a, Cstdc4, Gm48786, Gm19510, Gm32089, Gm5966, Stfa3, Tmem132e, Orm2, Gm28551, A630023A22Rik, Gm7457, Serpina7, Bmerb1, Klk9, 1700011B04Rik, Asprv1, Dmbt1, Tmprss11d, Slc5a7, Ifitm7, Gm38340, Pglyrp3, Gm26748, 4930431P03Rik, 4732490B19Rik, Sectm1b, Gm4948, Gm20406, Ras110b, Gm36079, Sult2a3, Apcs, Mymk, Nkx2-9, Gm26535, Rnase2a, Slc22a20, Stfa211, Gm4949, Gm11738, Gm29686, Serpine3, Gm807, Gm26444, Trdv5, Sultle1, Serpina3e-ps, 4930578G10Rik, Gm37645, Ceacam19, Ackr1, Ms4a3, B130046B21Rik, Ctcflos, Doc2b, Gm49573, Gm38275, Tex44, Gpr22, Ceacam10, E030018B13Rik, Gm37131, Htr3a, Slc13a5, Nxf3, Gm49427, Pde6h, Ubd, Forlb, Sult2a5, Tat, Gm6610, Gm44292, Gm44165, Prss22, Gm11714, Gm49368, Gm48065, Aoah, Tctex1d1, Phox2a, Slc36a3, Sult2a1, Gm37347, Gm50083, Epx, Gm26965, Reg3g, Cela3b, Gm12503, Gpbar1, Dnah6, Fut2, Muc4, Gm43189, Angpt17, Pcdh8, Sftpa1, Mlana, Gm16350, Syt2, Gm17055, Ppy, Ccdc33, Gm33280, Gm45643, Lrit3, Sbk2, Slc6a19os, Slamf1, Gm5541, Fgf21, Gm49392, Slc6a16, Olr1, Gm35028, Slfn4, Gm20548, Gm40709, Speer4f2, Gm13363, Egr2, Gm44260, Marc1, Crygn, Lpo, Gabrp, Bpifc, Fgf18, Lncenc1, Prss27, Muc19, Gm39929, Gm2670, 5830487J09Rik, Olfr1442, Krt20, Gm19684, Gm33424, 4732419C18Rik, Gm37621, Creb313, Gm14319, Dnah10, Areg, Armc12, Eif4ebp3, Gdf6, Gm49037, Gm42793, Der13, Nptx2, Atp12a, Kcna7, Prg4, Ctla4, Gm44164, Mmp7, Gm44949, Slc17a8, Lamb3, Plekhs1, Trim17, Dusp8, 4930422C21Rik, Gm10727, Col27a1, Tacc2, Fat2, Lif, Klk1b1, Tsc22d1, Wnt8b, Grem1, Hesx1, Hhipl1, Ugtla5, Klk1b4, Gm8947, A530013C23Rik, Mylk4, Cyp2a5, Muc1, Gm44588, Gm45053, IL-1b, IL-10, Tnf-a, Ifn-g, Nos1, Sele, Sell, Selp, Acat3, Tnfrsf1b, Csf1, Cx3cl1, and Ptgs2, Ccl12, Ccr2,Ccr5, Cxcl11, and/or Isg15.

4. The method of claim 2, wherein the expression of the one or more genes decreases during microbial inflammation and wherein the one or more genes are selected from the group consisting of Ano5, Gsg11, Spon2, Oxtr, Rnf112, Ppp2r2c, Crabp1, St6galnac3, Agtr2, Gjc2, Gm33195, Gabra4, Aplnr, E030013I19Rik, Pcdh12, Chp2, Gng8, Pla2g2d, Gm20683, Mansc4, Gm10824, Ces2b, Kank4, Zmat4, Cdr1os, Wnt2, Gm17040, Wfdc16, Rab42, Tbxa2r, Plppr5, Misp3, Mmp28, Lrrn4, Sost, Tmem200a, Shisa2, Stmn2, Chrm1, Tmeff2, 2810433D01Rik, Pabpc41, Dact3, Mett124, Gpr34, D430019H16Rik, Hoxc5, Tekt5, Megf10, Wscd1, Gm32219, Tril, Agtr1b, 2610203C22Rik, Abcg3, Star, Grik2, Grin2c, Vstm5, Smad9, Pianp, Rnf144a, Cthrc1, Nxpe4, Mrc2, Cx3cr1, Il5ra, Kcnc4, Fam163a, Cxcr3, A330074K22Rik, Gm11627, Mmp11, Fam181b, Cd209b, Tnfrsf19, Sept4, Nlgn3, Baalc, Ngb, Nxph3, Klb, Igkv4-72, Ptprv, Nrep, Itga8, Myct1, Palm, Syt3, Tspan18, Piezo2, Nat8f3, Cngb1, Cd209g, Faim2, Gfra2, Srpk3, Gm3716, Klhl13, Mogat2, Tmem233, Duxb13, Rnase1, Defb42, AL935121.1, Zp1, Snurf, Gm25541, 4933403008Rik, 1810046K07Rik, Gm5639, Myh2, Gm765, Gm50242, Gm6018, Ackr4, Tnfrsf17, Gm37858, Igkv14-130, Ptprh, Gpr165, Gm47652, Gm16534, 0610012D04Rik, Gm36569, Gzmk, Gm9918, Gm33677, St6gal2, Krtap17-1, Olfr1342, Lhcgr, Ccdc42, Zfp985, Igkv4-70, Gm31517, Gm9767, Olfr71, Slc26a3, Trpv3, Gm49727, Gm43951, E330018M18Rik, Calcb, Rspo2, Glp1r, Fam124b, Amtn, 4930570G19Rik, Gm11574, Pla2g2e, Sfrp2, Pigz, Gm13688, Abca14, B530045E10Rik, Fras1, Gm12159, Gm48488, 9030404E10Rik, Hmcn1, Igkv4-68, Wnt7a, Tmem74, Ighv1-66, Cr2, Slc5a11, Lrrc17, Slc22a14, Dipk2b, Fgf18, Ndp, Gm17334, Btn12, Gm10432, Colq, Gm48662, 4833412C05Rik, Gm12576, Igkv6-17, Gm13816, Tmem266, Ighv5-6, Cntn4, Rbp7, Insyn2a, Slc5a12, Gm9947, Igkv12-89, Gm26795, Ddx4, Dclk3, Hpcal4, Ndnf, Igkv17-121, Slurp1, Ano4, Ighv1-7, Rimklb, Igkv4-80, Cyp11b1, Cyp11a1, Akr1cl, Hsd3b1, Scg2, Th, Cyp21a1, Chga, Cyp21a2-ps, Pnmt, Chgb, Cyp11b2, Caln1, Kcnk9, Dbh, Gm28382, Pla2g2f, Nr5a1, Pcsk1n, Mgarp, 4930486L24Rik, Cryba4, Atp2b3, Resp18, E330017L17Rik, Pcsk1, Snap25, Rims4, Slc24a2, Phox2b, Slc18a2, Eef1a2, Chrna3, Hand2os1, Cntnap5a, Rasgrf1, Hand2, Pnmal1, Necab2, Tbx20, Stmn3, Gm17322, St8sia3, Rab33a, Synpr, Rit2, Camkv, Gdap1, Soga3, Mdga2, Mab2112, Nrsn1, Cadps, Gnb3, 1520401A03Rik, Nrg3, Spock1, Cdk5r2, Ina, Actl6b, Npy2r, Gal, Nrsn2, Gabrg2, Gpr158, Slc7a14, Sphkap, Tmem255a, 8430419K02Rik, Scg3, Gria1, Txndc2, Gm27477, Gria2, Gjd2, Lrrtm2, Hsd3b6, Diras1, Omg, Gm49391, Fam155a, Gm2007, Kcnq2, Gm42372, Is11, Lingo3, Pr18a2, Adh7, Lgi1, Gm47815, Gm14592, Cdx4, Marcks11-ps4, Sstr3, Pcdh10, Gm27000, Tub, Mc2r, I120ra, and/or Grin2a.

5. The method of claim 1, wherein the microbial inflammation is acute inflammation, subacute inflammation, chronic inflammation, organ-specific inflammation, systemic inflammation, sepsis, or polymicrobial sepsis.

6. The method of claim 1, wherein the NTCI comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

7. The method of claim 1, wherein the microbial inflammation is localized in the blood, brain, sinuses, upper respiratory tract, or lungs, heart, bone marrow, spleen, liver, kidneys, genito-urinary tract, bladder, aural cavities, stomach, intestines, skin, eyes, teeth, and/or gingiva.

8. The method of claim 7, wherein the microbial inflammation is localized in the lungs; wherein the expression of the one or more genes increases during microbial inflammation; and wherein the one or more genes are selected from the group consisting of I110, Acod1, Saa3, Gpr31b, Fgf23, Igfbp1, Mrgpra2b, Ccl3, Ccl4, Gpr84, Adamts4, Madcam1Olfm4, Il1f9, Gpr31a, Gpr31c, Ptx3, Slc7a11, Gm6553, Tmprss11g, Tac1, Il1f6, Ccl2, Psg16, Fos11, Sprr2a2, Serpina3m, Il1rn, Cc120, Tnf, Gm43181, Clec4e, Gm45774, Ugtla10, Kcnt1, Mt1, Mefv, Clec4d, Ms4a4a, Btbd16, Spic, Orm1, Serpina3f, Il1b, Serpina10, 9830107B12Rik, Gm49839, Msr1, Ankrd2, Saa4, S100a8, Hpx, Apoa4, Steap4, Cxcl10, S100a9, Wfdc17, Gnat1, Fgg, Fgb, Lilr4b, 2010003K11Rik, Serpina3n, AA467197, Gm16685, Timp1, Fga, Ifitm6, Lcn2, F10, Leap2, Retnlg, Ccl7, Ikbke, Vcan, F13a1, Serpina3g, 1200007C13Rik, Ngp, Siglece, Lrrc25, Gm10309, Trem1, Slc39a14, Fgl1, 1127, Tcp10b, Serpina3i, Cxc19, A730049H05Rik, Gm8883, Fpr2, Saa2, Saa1, Prok2, B230303A05Rik, Gm43079, Gm49383, Cldn14, Gm15056, U90926, Stfa2, Prokr2, Lbx1, Cstdc5, Tarm1, Mrgpra2a, Cstdc4, Gm48786, Gm19510, Gm32089, Gm5966, Stfa3, Tmem132e, Orm2, Gm28551, A630023A22Rik, Gm7457, Serpina7, Bmerb1, Klk9, 1700011B04Rik, Asprv1, Dmbt1, Tmprss11d, Slc5a7, Ifitm7, Gm38340, Pglyrp3, Gm26748, 4930431P03Rik, 4732490B19Rik, Sectm1b, Gm4948, Gm20406, Ras110b, Gm36079, Sult2a3, Apcs, Mymk, Nkx2-9, Gm26535, Rnase2a, Slc22a20, Stfa211, Gm4949, Gm11738, Gm29686, Serpine3, Gm807, Gm26444, Trdv5, Sultle1, Serpina3e-ps, 4930578G10Rik, Gm37645, Ceacam19, Ackr1, Ms4a3, B130046B21Rik, Ctcflos, Doc2b, Gm49573, Gm38275, Tex44, Gpr22, Ceacam10, E030018B13Rik, Gm37131, Htr3a, Slc13a5, Nxf3, Gm49427, Pde6h, Ubd, Forlb, Sult2a5, Tat, Gm6610, Gm44292, Gm44165, Prss22, Gm11714, Gm49368, Gm48065, Aoah, Tctex1d1, Phox2a, Slc36a3, Sult2a1, Gm37347, Gm50083, IL-1b, IL-10, Tnf-a, Ifn-g, Nos1, Sele, Sell, Selp, Acat3, Tnfrsf1b, Csf1, Cx3cl1, and Ptgs2, Ccl12, Ccr2,Ccr5, Cxcl11, and/or Isg15.

9. The method of claim 7, wherein the microbial inflammation is localized in the lungs; wherein the expression of the one or more genes decreases during microbial inflammation and wherein the one or more genes are selected from the group consisting of Ano5, Gsg11, Spon2, Oxtr, Rnf112, Ppp2r2c, Crabp1, St6galnac3, Agtr2, Gjc2, Gm33195, Gabra4, Aplnr, E030013I19Rik, Pcdh12, Chp2, Gng8, Pla2g2d, Gm20683, Mansc4, Gm10824, Ces2b, Kank4, Zmat4, Cdr1os, Wnt2, Gm17040, Wfdc16, Rab42, Tbxa2r, Plppr5, Misp3, Mmp28, Lrrn4, Sost, Tmem200a, Shisa2, Stmn2, Chrm1, Tmeff2, 2810433D01Rik, Pabpc41, Dact3, Mett124, Gpr34, D430019H16Rik, Hoxc5, Tekt5, Megf10, Wscd1, Gm32219, Tril, Agtr1b, 2610203C22Rik, Abcg3, Star, Grik2, Grin2c, Vstm5, Smad9, Pianp, Rnf144a, Cthrc1, Nxpe4, Mrc2, Cx3cr1, I15ra, Kcnc4, Fam163a, Cxcr3, A330074K22Rik, Gm11627, Mmp11, Fam181b, Cd209b, Tnfrsf19, Sept4, Nlgn3, Baalc, Ngb, Nxph3, Klb, Igkv4-72, Ptprv, Nrep, Itga8, Myct1, Palm, Syt3, Tspan18, Piezo2, Nat8f3, Cngb1, Cd209g, Faim2, Gfra2, Srpk3, Gm3716, Klh113, Mogat2, Tmem233, Duxb13, Rnase1, Defb42, AL935121.1, Zp1, Snurf, Gm25541, 4933403008Rik, 1810046K07Rik, Gm5639, Myh2, Gm765, Gm50242, Gm6018, Ackr4, Tnfrsf17, Gm37858, Igkv14-130, Ptprh, Gpr165, Gm47652, Gm16534, 0610012D04Rik, Gm36569, Gzmk, Gm9918, Gm33677, St6gal2, Krtap17-1, Olfr1342, Lhcgr, Ccdc42, Zfp985, Igkv4-70, Gm31517, Gm9767, Olfr71, Slc26a3, Trpv3, Gm49727, Gm43951, E330018M18Rik, Calcb, Rspo2, Glp1r, Fam124b, Amtn, 4930570G19Rik, Gm11574, Pla2g2e, Sfrp2, Pigz, Gm13688, Abca14, B530045E10Rik, Fras1, Gm12159, Gm48488, 9030404E10Rik, Hmcn1, Igkv4-68, Wnt7a, Tmem74, Ighv1-66, Cr2, Slc5a11, Lrrc17, Slc22a14, Dipk2b, Fgf18, Ndp, Gm17334, Btn12, Gm10432, Colq, Gm48662, 4833412C05Rik, Gm12576, Igkv6-17, Gm13816, Tmem266, Ighv5-6, Cntn4, Rbp7, Insyn2a, Slc5a12, Gm9947, Igkv12-89, Gm26795, Ddx4, Dclk3, Hpcal4, Ndnf, Igkv17-121, Slurp1, Ano4, Ighv1-7, Rimklb, and/or Igkv4-80.

10. The method of claim 7, wherein the microbial inflammation is localized in the kidneys; wherein the expression of the one or more genes increases during microbial inflammation and wherein the one or more genes are selected from the group consisting of Acod1, Saa3, Gpr31b, Fgf23, Igfbp1, Mrgpra2b, Ccl3, Ccl4, Gpr84, Adamts4, Madcam1, Olfm4, Il1f9, Gpr31a, Gpr31c, Ptx3, Slc7a11, Gm6553, Tmprss11g, Tac1, Il1f6, Ccl2, Psg16, Sprr2a2, Serpina3m, Il1rn, Ccl20, Tnf, Gm43181, Clec4e, Gm45774, Ugt1a10, Kcnt1, Mt1, Mefv, Clec4d, Ms4a4a, Btbd16, Spic, Orm1, Serpina3f, Il1b, Serpina10, 9830107B12Rik, Gm49839, Msr1, Ankrd2, Saa4, S100a8, Hpx, Apoa4, Steap4, Cxcl10, S100a9, Wfdc17, Gnat1, Fgg, Fgb, Lilr4b, 2010003K11Rik, Serpina3n, AA467197, Gm16685, Timp1, Fga, Ifitm6, Lcn2, F10, Leap2, Retnlg, Ccl7, Ikbke, Vcan, F13a1, Serpina3g, 1200007C13Rik, Ngp, Siglece, Lrrc25, Gm10309, Trem1, Slc39a14, Fgl1, 1127, Tcp10b, Serpina3i, Cxc19, A730049H05Rik, Gm8883, Fpr2, Epx, Gm26965, Reg3g, Cela3b, Gm12503, Gpbar1, Dnah6, Fut2, Muc4, Gm43189, Angpt17, Pcdh8, Sftpa1, Mlana, Gm16350, Syt2, Gm17055, Ppy, Ccdc33, Gm33280, Gm45643, Lrit3, Sbk2, Slc6a19os, Slamf1, Gm5541, Fgf21, Gm49392, Slc6a16, Olr1, Gm35028, Slfn4, Gm20548, Gm40709, Speer4f2, Gm13363, Egr2, Gm44260, Marc1, Crygn, Lpo, Gabrp, Bpifc, Fgf18, Lncenc1, Prss27, Muc19, Gm39929, Gm2670, 5830487J09Rik, Olfr1442, Krt20, Gm19684, Gm33424, 4732419C18Rik, Gm37621, Creb313, Gm14319, Dnah10, Areg, Armc12, Eif4ebp3, Gdf6, Gm49037, Gm42793, Derl3, Nptx2, Atp12a, Kcna7, Prg4, Ctla4, Gm44164, Mmp7, Gm44949, Slc17a8, Lamb3, Plekhs1, Trim17, Dusp8, 4930422C21Rik, Gm10727, Col27a1, Tacc2, Fat2, Lif, Klk1b1, Tsc22d1, Wnt8b, Grem1, Hesx1, Hhipl1, Ugt1a5, Klk1b4, Gm8947, A530013C23Rik, Mylk4, Cyp2a5, Muc1, Gm44588, Gm45053, IL-1b, IL-10, Tnf-a, Ifn-g, Nos1, Sele, Sell, Selp, Acat3, Tnfrsf1b, Csf1, Cx3cl1, and Ptgs2, Ccl12, Ccr2,Ccr5, Cxcl11, and/or Isg15.

11. The method of claim 7, wherein the microbial inflammation is localized in the kidneys; wherein the expression of the one or more genes decreases during microbial inflammation and wherein the one or more genes are selected from the group consisting of Ano5, Gsg11, Spon2, Oxtr, Rnf112, Ppp2r2c, Crabp1, St6galnac3, Agtr2, Gjc2, Gm33195, Gabra4, Aplnr, E030013I19Rik, Pcdh12, Chp2, Gng8, Pla2g2d, Gm20683, Mansc4, Gm10824, Ces2b, Kank4, Zmat4, Cdr1os, Wnt2, Gm17040, Wfdc16, Rab42, Tbxa2r, Plppr5, Misp3, Mmp28, Lrrn4, Sost, Tmem200a, Shisa2, Stmn2, Chrm1, Tmeff2, 2810433D01Rik, Pabpc41, Dact3, Mett124, Gpr34, D430019H16Rik, Hoxc5, Tekt5, Megf10, Wscd1, Gm32219, Tril, Agtr1b, 2610203C22Rik, Abcg3, Star, Grik2, Grin2c, Vstm5, Smad9, Pianp, Rnf144a, Cthrc1, Nxpe4, Mrc2, Cx3cr1, Il5ra, Kcnc4, Fam163a, Cxcr3, A330074K22Rik, Gm11627, Mmp11, Fam181b, Cd209b, Tnfrsf19, Sept4, Nlgn3, Baalc, Ngb, Nxph3, Klb, Igkv4-72, Ptprv, Nrep, Itga8, Myct1, Palm, Syt3, Tspan18, Piezo2, Nat8f3, Cngb1, Cd209g, Faim2, Gfra2, Srpk3, Gm3716, Klhl13, Mogat2, Cyp11b1, Cyp11a1, Akr1cl, Hsd3b1, Scg2, Th, Cyp21a1, Chga, Cyp21a2-ps, Pnmt, Chgb, Cyp11b2, Caln1, Kcnk9, Dbh, Gm28382, Pla2g2f, Nr5a1, Pcskln, Mgarp, 4930486L24Rik, Cryba4, Atp2b3, Resp18, E330017L17Rik, Pcsk1, Snap25, Rims4, Slc24a2, Phox2b, Slc18a2, Eef1a2, Chrna3, Hand2os1, Cntnap5a, Rasgrf1, Hand2, Pnmal1, Necab2, Tbx20, Stmn3, Gm17322, St8sia3, Rab33a, Synpr, Rit2, Camkv, Gdap1, Soga3, Mdga2, Mab2112, Nrsn1, Cadps, Gnb3, 1520401A03Rik, Nrg3, Spock1, Cdk5r2, Ina, Act16b, Npy2r, Gal, Nrsn2, Gabrg2, Gpr158, Slc7a14, Sphkap, Tmem255a, 8430419K02Rik, Scg3, Gria1, Txndc2, Gm27477, Gria2, Gjd2, Lrrtm2, Hsd3b6, Diras1, Omg, Gm49391, Fam155a, Gm2007, Kcnq2, Gm42372, Is11, Lingo3, Prl8a2, Adh7, Lgi1, Gm47815, Gm14592, Cdx4, Marcks11-ps4, Sstr3, Pcdh10, Gm27000, Tub, Mc2r, I120ra, and/or Grin2a.

12. The method of claim 1, wherein the microbial inflammation is caused by a microbe selected from the group consisting of a virus, a bacteria, a fungus, and a parasite viral infection.

13.-19. (canceled)

20. A method of reducing the microbial burden in the blood, brain, sinuses, upper respiratory tract, or lungs, heart, bone marrow, spleen, liver, kidneys, genito-urinary tract, bladder, aural cavities, stomach, intestines, skin, eyes, teeth, or gingiva of a subject comprising administering to the subject a therapeutically effective amount of a composition comprising an NTCI.

21. The method of claim 20, wherein the NTCI comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

22. The method of claim 20, wherein the method further comprises the administration of an anti-microbial agent.

23. The method of claim 20, wherein the microbial burden is caused by a microbe selected from the group consisting of a virus, a bacteria, a fungus, and a parasite.

24.-30. (canceled)


SEQUENCE LISTING (42 sequences)

SEQ ID NO: 1 - protein, 29 aa, synthetic construct
  REGION 1..29: note = Description of sequence: cSN50
  VARIANT 1: note = x is any amino acid
  VARIANT 2: note = x is any amino acid
  VARIANT 3: note = x is any amino acid
  VARIANT 4: note = x is any amino acid
  VARIANT 8: note = x is any amino acid
  VARIANT 9: note = x is any amino acid
  VARIANT 17: note = x is any amino acid
  VARIANT 18: note = x is any amino acid
  VARIANT 19: note = x is any amino acid
  VARIANT 26: note = x is any amino acid
  VARIANT 27: note = x is any amino acid
  VARIANT 28: note = x is any amino acid
  VARIANT 29: note = x is any amino acid
  VARIANT 1: note = This position may be absent
  VARIANT 2: note = This position may be absent
  VARIANT 3: note = This position may be absent
  VARIANT 4: note = This position may be absent
  VARIANT 8: note = This position may be absent
  VARIANT 9: note = This position may be absent
  VARIANT 17: note = This position may be absent
  VARIANT 18: note = This position may be absent
  VARIANT 19: note = This position may be absent
  VARIANT 26: note = This position may be absent
  VARIANT 27: note = This position may be absent
  VARIANT 28: note = This position may be absent
  VARIANT 29: note = This position may be absent
XXXXLLPXXLLALLAPXXXQRKRQKXXXX

SEQ ID NO: 2 - protein, 29 aa, synthetic construct
  REGION 1..29: note = Description of sequence: cSN50
  VARIANT 1: note = x is any amino acid
  VARIANT 2: note = x is any amino acid
  VARIANT 3: note = x is any amino acid
  VARIANT 4: note = x is any amino acid
  VARIANT 8: note = x is any amino acid
  VARIANT 9: note = x is any amino acid
  VARIANT 17: note = x is any amino acid
  VARIANT 18: note = x is any amino acid
  VARIANT 19: note = x is any amino acid
  VARIANT 26: note = x is any amino acid
  VARIANT 27: note = x is any amino acid
  VARIANT 28: note = x is any amino acid
  VARIANT 29: note = x is any amino acid
  VARIANT 1: note = This position may be absent
  VARIANT 2: note = This position may be absent
  VARIANT 3: note = This position may be absent
  VARIANT 4: note = This position may be absent
  VARIANT 8: note = This position may be absent
  VARIANT 9: note = This position may be absent
  VARIANT 17: note = This position may be absent
  VARIANT 18: note = This position may be absent
  VARIANT 19: note = This position may be absent
  VARIANT 26: note = This position may be absent
  VARIANT 27: note = This position may be absent
  VARIANT 28: note = This position may be absent
  VARIANT 29: note = This position may be absent
XXXXLLPXXLLAVLAPXXXQRKRQKXXXX

SEQ ID NO: 3 - protein, 29 aa, synthetic construct
  REGION 1..29: note = Description of sequence: cSN50
  VARIANT 1: note = x is any amino acid
  VARIANT 2: note = x is any amino acid
  VARIANT 3: note = x is any amino acid
  VARIANT 4: note = x is any amino acid
  VARIANT 8: note = x is any amino acid
  VARIANT 9: note = x is any amino acid
  VARIANT 17: note = x is any amino acid
  VARIANT 18: note = x is any amino acid
  VARIANT 19: note = x is any amino acid
  VARIANT 26: note = x is any amino acid
  VARIANT 27: note = x is any amino acid
  VARIANT 28: note = x is any amino acid
  VARIANT 29: note = x is any amino acid
  VARIANT 1: note = This position may be absent
  VARIANT 2: note = This position may be absent
  VARIANT 3: note = This position may be absent
  VARIANT 4: note = This position may be absent
  VARIANT 8: note = This position may be absent
  VARIANT 9: note = This position may be absent
  VARIANT 17: note = This position may be absent
  VARIANT 18: note = This position may be absent
  VARIANT 19: note = This position may be absent
  VARIANT 26: note = This position may be absent
  VARIANT 27: note = This position may be absent
  VARIANT 28: note = This position may be absent
  VARIANT 29: note = This position may be absent
XXXXLLPXXLLAVLAPXXXQRDEQKXXXX

SEQ ID NO: 4 - protein, 29 aa, synthetic construct
  REGION 1..29: note = Description of sequence: cSN50
  VARIANT 1: note = x is any amino acid
  VARIANT 2: note = x is any amino acid
  VARIANT 3: note = x is any amino acid
  VARIANT 4: note = x is any amino acid
  VARIANT 8: note = x is any amino acid
  VARIANT 9: note = x is any amino acid
  VARIANT 18: note = x is any amino acid
  VARIANT 19: note = x is any amino acid
  VARIANT 26: note = x is any amino acid
  VARIANT 27: note = x is any amino acid
  VARIANT 28: note = x is any amino acid
  VARIANT 1: note = This position may be absent
  VARIANT 2: note = This position may be absent
  VARIANT 3: note = This position may be absent
  VARIANT 4: note = This position may be absent
  VARIANT 8: note = This position may be absent
  VARIANT 9: note = This position may be absent
  VARIANT 18: note = This position may be absent
  VARIANT 19: note = This position may be absent
  VARIANT 26: note = This position may be absent
  VARIANT 27: note = This position may be absent
  VARIANT 28: note = This position may be absent
XXXXLLPXXLLALLAPCXXQRKRQKXXXC

SEQ ID NO: 5 - protein, 28 aa, synthetic construct
  REGION 1..28: note = Description of sequence: cSN50
  VARIANT 1: note = x is any amino acid
  VARIANT 2: note = x is any amino acid
  VARIANT 3: note = x is any amino acid
  VARIANT 4: note = x is any amino acid
  VARIANT 8: note = x is any amino acid
  VARIANT 9: note = x is any amino acid
  VARIANT 18: note = x is any amino acid
  VARIANT 25: note = x is any amino acid
  VARIANT 26: note = x is any amino acid
  VARIANT 27: note = x is any amino acid
  VARIANT 1: note = This position may be absent
  VARIANT 2: note = This position may be absent
  VARIANT 3: note = This position may be absent
  VARIANT 4: note = This position may be absent
  VARIANT 8: note = This position may be absent
  VARIANT 9: note = This position may be absent
  VARIANT 18: note = This position may be absent
  VARIANT 25: note = This position may be absent
  VARIANT 26: note = This position may be absent
  VARIANT 27: note = This position may be absent
XXXXLLPXXLLALLAPCXQRKRQKXXXC

SEQ ID NO: 6 - protein, 29 aa, synthetic construct
  REGION 1..29: note = Description of sequence: cSN50
AAVALLPAVLLALLAPCYVQRKRQKLMPC

SEQ ID NO: 7 - protein, 28 aa, synthetic construct
  REGION 1..28: note = Description of sequence: cSN50
AAVALLPAVLLALLAPCVQRKRQKLMPC

SEQ ID NO: 8 - protein, 28 aa, synthetic construct
  REGION 1..28: note = Description of sequence: cSN50
AAVALLPAVLLAVLAPCVQRKRQKLMPC

SEQ ID NO: 9 - protein, 28 aa, synthetic construct
  REGION 1..28: note = Description of sequence: cSN50
AAVALLPAVLLAVLAPCVQRDEQKLMPC

SEQ ID NO: 10 - protein, 10 aa, synthetic construct
  REGION 1..10: note = Description of sequence: Nuclear transport checkpoint inhibitor : N50
VQRKRQKLMP

SEQ ID NO: 11 - protein, 10 aa, synthetic construct
  REGION 1..10: note = Description of sequence: Nuclear transport checkpoint inhibitor : N50M
VQRDEQKLMP

SEQ ID NO: 12 - protein, 12 aa, synthetic construct
  REGION 1..12: note = Description of sequence: Nuclear transport checkpoint inhibitor : cN50.1
CVQRKRQKLMPC

SEQ ID NO: 13 - protein, 26 aa, synthetic construct
  REGION 1..26: note = Description of sequence: Nuclear transport checkpoint inhibitor : SN50
AAVALLPAVLLALLAPVQRKRQKLMP

SEQ ID NO: 14 - protein, 7 aa, synthetic construct
  REGION 1..7: note = Description of sequence: Nuclear transport checkpoint inhibitor : SSHR?1
AAVALLP

SEQ ID NO: 15 - protein, 9 aa, synthetic construct
  REGION 1..9: note = Description of sequence: Nuclear transport checkpoint inhibitor : SSHR?2
AVLLALLAP

SEQ ID NO: 16 - protein, 28 aa, synthetic construct
  REGION 1..28: note = Description of sequence: Peptide name: cSN50.1b: Imp beta-selective (cell culture and preclinical studies)
AAVALLPAVLLALLAPCVQRDEQKLMPC

SEQ ID NO: 17 - protein, 28 aa, synthetic construct
  REGION 1..28: note = Description of sequence: Peptide name: cMN50.1: Imp alpha 5-selective
AAVALLPAVXLAXXAPCVQRKRQKLMPC

SEQ ID NO: 18 - protein, 32 aa, synthetic construct
  REGION 1..32: note = Description of sequence: Peptide name: SIBB: Inhibitor of Imp alpha 1-importin beta interaction
AAVALLPAVLLALLAPRRRRIEVNVELRKAKK

SEQ ID NO: 19 - protein, 34 aa, synthetic construct
  REGION 1..34: note = Description of sequence: Peptide name: SI-1: Inhibitor of Imp alpha 1-importin beta interaction
AAVALLPAVLLALLAPRRRRIEVNVELRKAKKDD

SEQ ID NO: 20 - protein, 34 aa, synthetic construct
  REGION 1..34: note = Description of sequence: Peptide name: SI-3: Inhibitor of Imp alpha 3-importin beta interaction
AAVALLPAVLLALLAPRRQRNEVVVELRKNKRDE

SEQ ID NO: 21 - protein, 34 aa, synthetic construct
  REGION 1..34: note = Description of sequence: Peptide name: SI-4: Inhibitor of Imp alpha 4-importin beta interaction
AAVALLPAVLLALLAPRRHRNEVTVELRKNKRDE

SEQ ID NO: 22 - protein, 34 aa, synthetic construct
  REGION 1..34: note = Description of sequence: Peptide name: SI-5: Inhibitor of Imp alpha 5-importin beta interaction
AAVALLPAVLLALLAPRRRREEEGLQLRKQKREE

SEQ ID NO: 23 - protein, 34 aa, synthetic construct
  REGION 1..34: note = Description of sequence: Peptide name: SI-7: Inhibitor of Imp alpha 7-importin beta interaction
AAVALLPAVLLALLAPRRRREEEGIQLRKQKREQ

SEQ ID NO: 24 - protein, 28 aa, synthetic construct
  REGION 1..28: note = Description of sequence: Peptide name: cSIB: Inhibitor of Imp alpha 1-importin beta interaction
AAVALLPAVLLALLAPCTEMRRRRIEVC

SEQ ID NO: 25 - protein, 37 aa, synthetic construct
  REGION 1..37: note = Description of sequence: Peptide name: SAR1: Imp alpha 1-specific
AAVALLPAVLLALLAPVELRKAKKDDQMLKRRNVSSF

SEQ ID NO: 26 - protein, 37 aa, synthetic construct
  REGION 1..37: note = Description of sequence: Peptide name: SAR3: Imp alpha 3-specific
AAVALLPAVLLALLAPVELRKNKRDEHLLKRRNVPHE

SEQ ID NO: 27 - protein, 37 aa, synthetic construct
  REGION 1..37: note = Description of sequence: Peptide name: SAR4: Imp alpha 4-specific
AAVALLPAVLLALLAPVELRKNKRDEHLLKKRNVPQE

SEQ ID NO: 28 - protein, 37 aa, synthetic construct
  REGION 1..37: note = Description of sequence: Peptide name: SAR5: Imp alpha 5-specific
AAVALLPAVLLALLAPLQLRKQKREEQLFKRRNVATA

SEQ ID NO: 29 - protein, 37 aa, synthetic construct
  REGION 1..37: note = Description of sequence: Peptide name: SAR7: Imp alpha 7-specific
AAVALLPAVLLALLAPIQLRKQKREQQLFKRRNVELI

SEQ ID NO: 30 - protein, 29 aa, synthetic construct
  REGION 1..29: note = Description of sequence: Peptide name: cSAR1-C: Imp alpha 1-specific
AAVALLPAVLLALLAPCVELRKAKKDDQC

SEQ ID NO: 31 - protein, 29 aa, synthetic construct
  REGION 1..29: note = Description of sequence: Peptide name: cSAR3-C: Imp alpha 3-specific
AAVALLPAVLLALLAPCVELRKNKRDEHC

SEQ ID NO: 32 - protein, 29 aa, synthetic construct
  REGION 1..29: note = Description of sequence: Peptide name: cSAR5-C: Imp alpha 5-specific
AAVALLPAVLLALLAPCLQLRKQKREEQC

SEQ ID NO: 33 - protein, 29 aa, synthetic construct
  REGION 1..29: note = Description of sequence: Peptide name: cSAR7-C: Imp alpha 7-specific
AAVALLPAVLLALLAPCIQLRKQKREQQC

SEQ ID NO: 34 - protein, 29 aa, synthetic construct
  REGION 1..29: note = Description of sequence: Peptide name: cSAR1-N: Imp alpha 1-specific
AAVALLPAVLLALLAPCQMLKRRNVSSFC

SEQ ID NO: 35 - protein, 29 aa, synthetic construct
  REGION 1..29: note = Description of sequence: Peptide name: cSAR3-N: Imp alpha 3-specific
AAVALLPAVLLALLAPCHLLKRRNVPHEC

SEQ ID NO: 36 - protein, 29 aa, synthetic construct
  REGION 1..29: note = Description of sequence: Peptide name: cSAR4-N: Imp alpha 4-specific
AAVALLPAVLLALLAPCHLLKKRNVPQEC

SEQ ID NO: 37 - protein, 29 aa, synthetic construct
  REGION 1..29: note = Description of sequence: Peptide name: cSAR5-N: Imp alpha 5-specific
AAVALLPAVLLALLAPCQLFKRRNVATAC

SEQ ID NO: 38 - protein, 29 aa, synthetic construct
  REGION 1..29: note = Description of sequence: Peptide name: cSAR7-N: Imp alpha 7-specific
AAVALLPAVLLALLAPCQLFKRRNVELIC

SEQ ID NO: 39 - protein, 37 aa, synthetic construct
  REGION 1..37: note = Description of sequence: Nuclear transport checkpoint inhibitor
  VARIANT 10: note = x is any amino acid
  VARIANT 13: note = x is any amino acid
  VARIANT 14: note = x is any amino acid
  VARIANT 10: note = This position may be absent
  VARIANT 13: note = This position may be absent
  VARIANT 14: note = This position may be absent
AAVALLPAVXLAXXAPVELRKNKRDEHLLKRRNVPHE

SEQ ID NO: 40 - protein, 10 aa, synthetic construct
  REGION 1..10: note = Description of sequence: Peptide name: SM12: Importin beta-selective inhibitor (binding studies)
VQRDEQKLMP

SEQ ID NO: 41 - skipped

SEQ ID NO: 42 - protein, 33 aa, synthetic construct
  REGION 1..33: note = Description of sequence: Labeled Nuclear transport checkpoint inhibitor
  MOD_RES 18: note = Fluorescein isothiocyanate (FITC) labled amnio acid
AAVALLPAVLLALLAGGKGGPCVQRKRQKLMPC